SEQUENCE LISTING (32 sequences)

SEQ ID NO: 1 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 2 - protein, 5 aa, Homo sapiens
HSSGC

SEQ ID NO: 3 - protein, 12 aa, Homo sapiens
KHSSGCAFLSVK

SEQ ID NO: 4 - protein, 14 aa, Homo sapiens
IDDHKKHSSGCAFL

SEQ ID NO: 5 - protein, 10 aa, Homo sapiens
KKHSSGCAFL

SEQ ID NO: 6 - protein, 6 aa, Homo sapiens
KHSSGC

SEQ ID NO: 7 - protein, 6 aa, Homo sapiens
HSSGCA

SEQ ID NO: 8 - protein, 7 aa, Homo sapiens
KHSSGCA

SEQ ID NO: 9 - protein, 7 aa, Homo sapiens
KKHSSGC

SEQ ID NO: 10 - protein, 7 aa, Homo sapiens
HSSGCAF

SEQ ID NO: 11 - protein, 15 aa, Homo sapiens
HKKHSSGCAFLSVKK

SEQ ID NO: 12 - protein, 9 aa, Homo sapiens
KHSSGCAFL

SEQ ID NO: 13 - protein, 9 aa, Homo sapiens
TSHHHHHHC

SEQ ID NO: 14 - protein, 66 aa, Homo sapiens
RLKTFANFPSGSPVSASTLARAGFLYTGEGDTVRCFSCHAAVDRWQYGDSAVGRHRKVSPNCRFIN

SEQ ID NO: 15 - protein, 66 aa, Homo sapiens
RMSTYSTFPAGVPVSERSLARAGFYYTGVNDKVKCFCCGLMLDNWKRGDSPTEKHKKLYPSCRFVQ

SEQ ID NO: 16 - protein, 66 aa, Homo sapiens
RMSTYSTFPAGVPVSERSLARAGFYYTGVNDKVKCFCCGLMLDNWKLGDSPIQKHKQLYPSCSFIQ

SEQ ID NO: 17 - protein, 281 aa, Homo sapiens [sequence omitted]

SEQ ID NO: 18 - protein, 16 aa, Drosophila melanogaster
RQIKIWFQNRRMKWKK

SEQ ID NO: 19 - protein, 9 aa, Human immunodeficiency virus
RKKRRQRRR

SEQ ID NO: 20 - protein, 10 aa, Artificial Sequence
  Synthetically generated peptide
RKKRRRRRGC

SEQ ID NO: 21 - DNA, 13 bp, Artificial Sequence
  Synthetic Oligonucleotide
ccgccaagaagcg

SEQ ID NO: 22 - DNA, 100 bp, Artificial Sequence
  Synthetic Oligonucleotide
gcgtgcacacgcgcgtagacttcccccgcaagtcactcgttagcccgccaagaagcgacccctccggggcgagctgagcggcgtggcgcgggggcgtcat

SEQ ID NO: 23 - DNA, 101 bp, Artificial Sequence
  Synthetic Oligonucleotide
acgtgcatacgcacgtagacattccccgcttcccactccaaagtccgccaagaagcgtatcccgctgagcggcgtggcgcgggggcgtcatccgtcagctc

SEQ ID NO: 24 - DNA, 58 bp, Artificial Sequence
  Synthetic Oligonucleotide
acttcccccgcaagtcactcgttagcccgccaagaagcgacccctccggggcgagctg

SEQ ID NO: 25 - protein, 9 aa, Artificial Sequence
  Synthetically generated peptide
LFACGSSHK

SEQ ID NO: 26 - protein, 5 aa, Artificial Sequence
  Synthetically generated peptide
CGSSH

SEQ ID NO: 27 - protein, 5 aa, Artificial Sequence
  Synthetically generated peptide
GSSHK

SEQ ID NO: 28 - protein, 25 aa, Artificial Sequence
  Synthetically generated peptide
KKWKMRRNQFWVKVQRLFACGSSHK

SEQ ID NO: 29 - protein, 21 aa, Artificial Sequence
  Synthetically generated peptide
KKWKMRRNQFWVKVQRCGSSH

SEQ ID NO: 30 - protein, 21 aa, Artificial Sequence
  Synthetically generated peptide
KKWKMRRNQFWVKVQRGSSHK

SEQ ID NO: 31 - protein, 20 aa, Artificial Sequence
  Synthetically generated peptide
RRIVVLHGYGAVKEVLLNHK

SEQ ID NO: 32 - protein, 22 aa, Artificial Sequence
  Synthetically generated peptide
MLSLRQDIRFFKPATRTLCSSR

(19) United States
(12) Patent Application Publication
(10) Pub. No.: US 2011/0268722 A1
(43) Pub. Date: Nov. 3, 2011

(54) COMBINATION THERAPIES WITH MITOCHONDRIAL-TARGETED ANTI-TUMOR AGENTS

(76) Inventors: Markus D. Siegelin, Weehawken, NJ (US); Dario C. Altieri, Worcester, MA (US)

(21) Appl. No.: 13/092,475

(22) Filed: Apr. 22, 2011

Related U.S. Application Data
(60) Provisional application No. 61/326,872, filed on Apr. 22, 2010.

(51) Int. Cl. A61K 38/17; C12N 5/071; A61P 35/00; A61K 39/395; C07D 225/06

(52) U.S. Cl. 424/130.1; 514/18.9; 540/461; 435/375

(57) ABSTRACT

Described are mitochondria-targeted anti-tumor agents, death receptor agonists, autophagy inhibitors, and NF-κB signaling pathway inhibitors, and methods of making and using the same for the treatment of disorders associated with unwanted cell proliferation.

### CROSS-REFERENCE TO RELATED APPLICATIONS

This application claims priority to U.S. provisional application 61/326,872, filed Apr. 22, 2010, the contents of each of which are incorporated herein by reference in their entirety.

### TECHNICAL FIELD

This invention relates to the use of at least one mitochondria-targeted inhibitor of molecular chaperones in combination with one or more death receptor agonists, autophagy inhibitors, and/or NF-κB signaling pathway inhibitors for the treatment of disorders associated with unwanted cell proliferation.

### BACKGROUND

Tumor cells exhibit an enhanced ability to survive and proliferate in highly unfavorable environments. They have been shown to down-regulate many of the cellular pathways that prevent normal (i.e., non-cancerous) cells from dividing in a hostile environment, and they also inactivate apoptotic pathways that bring about cell death in many normal tissues under adverse conditions. Tumor cells are also believed to up-regulate pathways required to maintain active proliferation. For example, many tumor cells activate the cellular stress-response pathway that allows tumor cells to synthesize and maintain the protein machinery they need to continue proliferating. The activated stress response in tumors includes up-regulation of heat-shock proteins (Hsps), which are ATPase-directed molecular chaperones. In particular, Hsp90 is upregulated in many cancerous tissues. Hsp90 controls the balance between folding/maturation and proteasomal destruction of a restricted number of client proteins, some of which are involved in signal transduction and cell proliferation.

### SUMMARY

The present invention is based, at least in part, on the discovery that inhibition of molecular chaperones in tumor cell mitochondria using mitochondrial-targeted chaperone inhibitors potentiated the effect of death receptor agonists to induce cell death, e.g., in cancer cells, and that mitochondrial-targeted chaperone inhibitors induce autophagy and decrease NF-κB signaling pathway activity in cancer cells.

Provided herein are methods of enhancing apoptosis in one or more mammalian cells that include contacting the one or more cells with at least one mitochondrial-targeted chaperone inhibitor and at least one agent selected from the group of: a death receptor agonist, an autophagy inhibitor, and a NF-κB signaling pathway inhibitor, where the at least one mitochondrial-targeted chaperone inhibitor and the at least one agent are in an amount sufficient to enhance apoptosis in the one or more cells. In some embodiments, the one or more cells are contacted sequentially with the at least one mitochondrial-targeted chaperone inhibitor and the at least one agent. In some embodiments, the one or more cells are contacted simultaneously with the at least one mitochondrial-targeted chaperone inhibitor and the at least one agent.

Also provided are methods of treating of a proliferative disorder in a subject that include administering to the subject at least one mitochondrial-targeted chaperone inhibitor and at least one agent selected from the group of: a death receptor agonist, an autophagy inhibitor, and a NF-κB signaling pathway inhibitor, where the at least one mitochondrial-targeted chaperone inhibitor and the at least one agent are administered in an amount sufficient to treat of a proliferative disorder in the subject.

Also provided are methods for enhancing cancer or tumor cell death that include administering to the subject at least one mitochondrial-targeted chaperone inhibitor and at least one agent selected from the group of: a death receptor agonist, an autophagy inhibitor, and a NF-κB signaling pathway inhibitor, where the at least one mitochondrial-targeted chaperone inhibitor and the at least one agent are administered in an amount sufficient to enhance cancer or tumor cell death in a subject.

Some embodiments of the methods described herein further include identifying a subject having cancer or a tumor and/or determining whether cells of the cancer or tumor have increased mitochondrial concentrations of a chaperone as compared to a control cell. In some embodiments of the methods described herein, the at least one mitochondrial-targeted chaperone inhibitor and the at least one agent are administered simultaneously to the subject. In some embodiments of the methods described herein, the at least one agent is administered to the subject prior to the at least one mitochondrial-targeted chaperone inhibitor, or the at least one mitochondrial-targeted chaperone inhibitor is administered to the subject prior to the at least one agent. In some embodiments of all of the methods described herein, the proliferative disorder is a cancer (e.g., a cancer selected from the group of small-cell lung cancer, non-small cell lung cancer, colon cancer, colorectal cancer, and pancreatic cancer).

Also provided are methods of treating a therapeutic-resistant cancer in a subject that include administering to a subject having cancer cells resistant to a cancer therapeutic at least one mitochondrial-targeted chaperone inhibitor and the cancer therapeutic, where the at least one mitochondrial-targeted chaperone inhibitor and the cancer therapeutic are administered in amount sufficient to treat the therapeutic-resistant cancer. Some embodiments of these methods, further comprise identifying a subject as having a therapeutic-resistant cancer. In some embodiments, the subject is identified by detecting an increase in NF-κB signaling activity in a cancer cell in the subject.

Also provided is the use of at least one mitochondrial-targeted chaperone inhibitor for the preparation of a medicament for treatment of a proliferative disorder. Also provided is the use of at least one death receptor agonist, autophagy inhibitor, and NF-κB signaling pathway inhibitor in the preparation of a medicament for treatment of a proliferative disorder, characterized in that the treatment includes administering at least one mitochondrial-targeted chaperone inhibitor. Also provided is the use of at least one mitochondrial-targeted chaperone inhibitor in the preparation of a medicament for treatment of a proliferative disorder, characterized in that the treatment includes administering at least one death receptor agonist, autophagy inhibitor, and NF-κB signaling pathway inhibitor.

Also provided is a combination of at least one death receptor agonist, autophagy inhibitor, and NF-κB signaling pathway inhibitor and at least one mitochondrial-targeted chaperone inhibitor for the treatment of a proliferative disorder.

Also provided are compositions including at least one death receptor agonist, autophagy inhibitor, and NF-κB signaling pathway inhibitor and at least one mitochondrial-targeted chaperone inhibitor.

In any of the methods, uses, compositions, and kits described herein, the mitochondrial-targeted chaperone inhibitor is a composition comprising the formula:

A-B,

wherein A is a molecular chaperone inhibitor and B is a mitochondria-penetrating moiety and A and B are linked, optionally by a linking moiety, or a pharmaceutically acceptable salt thereof. In some embodiments of all of the methods, uses, compositions, and kits described herein, A is a small molecule selected from the group consisting of an Ansamycin class Hsp90 inhibitor; a geldanamycin analogue Hsp90 inhibitor; a purine-scaffold class Hsp90 inhibitor; a resorcinol Hsp90 inhibitor; and a macrolactone-Hsp90 inhibitor; or is a peptide inhibitor of Hsp90 or a Shepherdin peptide comprising SEQ ID NO:2 (His-Ser-Ser-Gly-Cys), or comprises 17-allylamino-demethoxygeldamycin (17-AAG), radicicol, a purine-scaffold class Hsp90 inhibitor, or 17-dimethylaminogeldanamycin. In some embodiments of all of the methods, uses, compositions, and kits described herein, A is a peptide inhibitor of Hsp90 that comprises a sequence that is at least 95% identical to SEQ ID NO:1 and binds to and inhibits Hsp90.

In some embodiments of all of the methods, uses, compositions, and kits described herein, B is selected from the group consisting of: a mitochondria penetrating peptide, an RNA mitochondrial penetrating signal, guanidine-rich peptoids, guanidine-rich polycarbamates, β-oligoarginines, and proline-rich dendrimers. In some embodiments of all of the methods, uses, compositions, and kits described herein, B is a mitochondria penetrating peptide selected from the group consisting of: a mitofusin peptide, a mitochondrial targeting signal peptide, TAT peptide, Antennapedia helix III homeodomain cell-penetrating peptide (ANT) peptide, VP22 peptide, and Pep-1 peptide.

In some embodiments of all of the methods, uses, compositions, and kits described herein, B is selected from the group of:

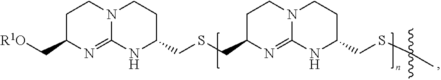

where:

R¹ is H, alkyl, alkenyl, alkynyl, haloalkyl, aryl, arylalkyl, or R^{a}R^{b}R^{c}Si;

R^{a}, R^{b}, and R^{c} are independently selected from alkyl or aryl; and

n is 0, 1, 2, 3, 4, 5, or 6;

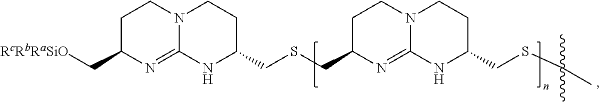

where:

R^{a}, R^{b}, and R^{c} are independently selected from alkyl or aryl; and

n is 1, 2, or 3;

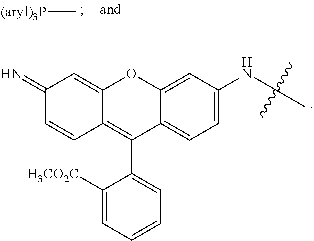

In some embodiments of any of the methods, uses, compositions, and kits described herein, A is

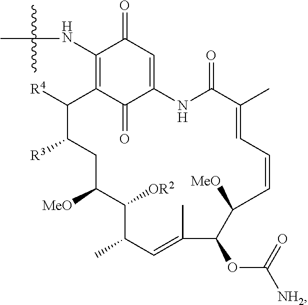

where:
R² is H, alkyl, aryl, or arylalkyl; R³ is H, alkyl; and R⁴ is H, alkyl, alkenyl, aryl, arylalkyl, OR^{d}, where R^{d} is H, alkyl, or arylalkyl. In some embodiments, R² is H or alkyl; R³ is H, alkyl; and R⁴ is H, or OR^{d}, wherein R^{d} is H, alkyl. In some embodiments, R² is H; R³ is methyl; and R⁴ is H.

In some embodiments of all of the above methods, uses, compositions, and kits, B contains ANT or mitochondrial-penetrating fragment thereof. In some embodiments of all of the above methods, uses, compositions, and kits, the composition includes a linking moiety between A and B (e.g., a linking moiety selected from the group of a peptide linker and a chemical linker). In some embodiments, the linker moiety is divalent and selected from the group consisting of alkylene, alkenylene, alkynylene, cycloalkylene, arylene, heteroarylene, and peptide linker, wherein any two adjacent carbon-carbon bonds of said alkylene, alkenylene, or alkynylene, can be optionally replaced with one or more of O, NH, S, PR^{e}, C(O)NR^{f}, arylene, heterocycloalkylene, or heteroarylene; wherein Re and Rf are independently selected from alkyl or aryl. In some embodiments, the linker moiety is

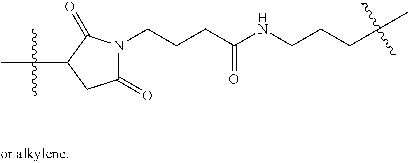

In some embodiments, the linker moiety is alkylene with six carbon atoms.

In some embodiments of any of the above methods, compositions, uses, and kits, A-B is:

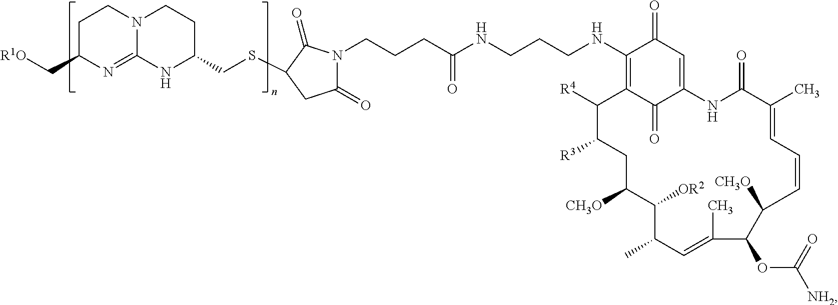

where, R¹ is H, alkyl, alkenyl, alkynyl, haloalkyl, aryl, arylalkyl, or R^{a}R^{b}R^{c}Si;
R² is H, alkyl, aryl, or arylalkyl; R³ is H, alkyl; R⁴ is H, alkyl, alkenyl, aryl, arylalkyl, OR^{d}, where R^{d} is H, alkyl, or arylalkyl;
R^{a}, R^{b}, and R^{c} are independently selected from alkyl or aryl; and
n is an integer between 1 and 10, inclusive; or a pharmaceutically acceptable salt thereof (e.g., a hexafluorophosphate salt). In some embodiments, R¹ is R^{a}R^{b}R^{c}Si wherein R^{a}, R^{b}, and R^{c} are independently selected from alkyl or aryl; R² is H; R³ is H, alkyl; R⁴ is H; and
n is 1, 2, 3, or 4.

In some embodiments of any of the methods, uses, compositions, or kits described herein, A-B is selected from the group of:

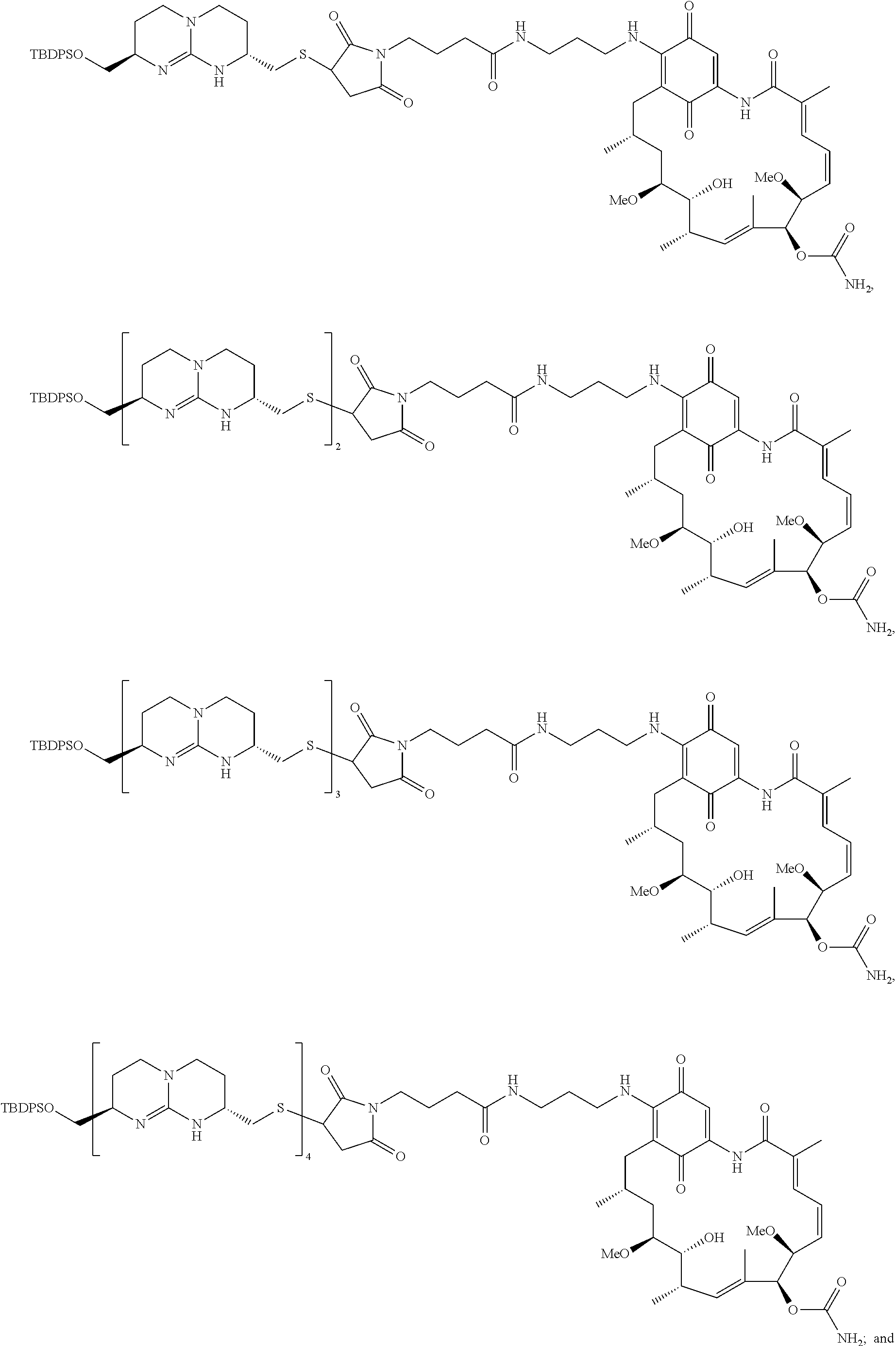

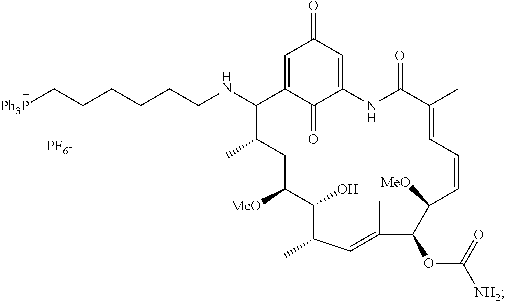

or a pharmaceutically acceptable salt thereof (e.g., a hexafluorophosphate salt).

In some embodiments of any of the methods, uses, compositions, and kits described herein, A-B is:

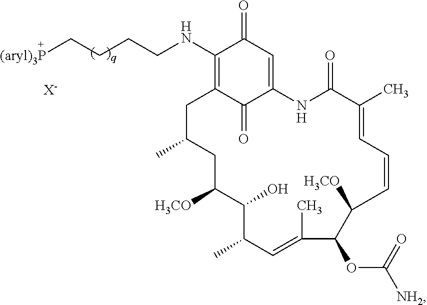

where, q is 1, 2, 3, 4, 5, or 6; and X is a pharmaceutically acceptable counter-ion. In some embodiments, q is 3. In some embodiments, the aryl is phenyl and q is 3. In some embodiments, X is hexafluorophosphate.

In any of the methods, uses, compositions, or kits described herein, the death receptor agonist is an agonist of tumor necrosis factor (TNF)-related apoptosis-inducing ligand receptor 1 (TRAILR1), TRAILR2, CD95, tumor necrosis factor receptor 1 (TNFR1), death receptor 3 (DR3), DR6, ectodysplasin A receptor (EDAR), or nerve growth factor receptor (NGFR). In some embodiments, the death receptor agonist is an agonist of TRAILR1 or TRAILR2. In some embodiments, the death receptor agonist contains an Apo2L/TRAIL polypeptide. In some embodiments, the Apo2L/TRAIL polypeptide is a fragment of the polypeptide of SEQ ID NO:17 (e.g., a fragment containing amino acids 114-281 of SEQ ID NO:17). In some embodiments, the Apo2L/TRAIL fragment contains amino acids 114-281 of SEQ ID NO:17 and is linked to one or more polyethylene glycol (PEG) molecules. In some embodiments, the death receptor agonist contains an antibody (e.g., mapatumumab, lexatumumab, conatumumab, or apomab).

In some embodiments of any of the above methods, uses, compositions, and kits, the autophagy inhibitor is selected from the group consisting of: 3-methyladenine, bafilomycin A1, LY294002, wortmanin, hydroxychloroquine, chloroquine, 5-amino-4-imidazole carboxamide riboside, okadaic acid, a microcystin, microeystin, nodularin, analogues of cAMP, agents that elevate cAMP levels, adenosine, N6-mercaptopurine riboside, wortmannin, vinblastine, an antisense oligonucleotide, ribozyme, or siRNA that decreases the expression of MAP1LC3B, HSP90AA1, HSPA8, AMBRA1, ATG12, ATG16L1, ATG4A, ATG4B, ATG4C, ATG4D, ATG5, ATG9A, ATG9B, BECN1, GABARAP, GABARAPL1, GABARAPL2, IRGM, MAP1LC3A, RGS1, ULK1, ATG10, ATG16L1, ATG16L2, ATG3, ATG7, RAB24, DRAM, TMEM166, ATG3, AKT1, APP, ATG12, BAD, BAK1, BAX, BCL2, BCL2L1, BID, BNIP3, CASP3, CASP8, CDKN1B, CDKN2A, CLN3, CTSB, CXCR4, DAPK1, DRAM, EIF2AK3, FADD, FAS, HDAC1, HTT, IFNA2, IFNG, IGF1, INS, MAPK8, NFKB1, PIK3CG, PRKAA1, PTEN, SNCA, SQSTM1, TGFB1, TGM2, TNF, TNFSF10, CDKN1B, CDKN2A, IFNG, PTEN, RB1, TGFB1, TP53, TP73, EIF2AK3, IFNA2, IFNA4, IFNG, ARSA, CTSS, EIF4G1, ESR1, GAA, HGS, MAPK14, PIK3C3, PIK3R4, PRKAA2, RPS6KB1, TMEM74, TMEM77, ULK2, and UVRAG.

In some embodiments, of any of the above methods, uses, compositions, or kits, the NF-κB signaling inhibitor reduces IκBα phosphorylation and/or degradation, NF-κB nuclear translocation, NF-κB binding to a κB promoter element, and/or transactivation of transcription of an NF-κB target gene.

“Cancer,” as the term is used herein, refers to a disease characterized by uncontrolled, abnormal growth of cells. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The methods described herein are useful for the treatment of cancer and tumor cells, i.e., both malignant and benign tumors as well as cancers with no solid tumors (such as hematopoietic cancers), so long as the cells to be treated have mitochondrial localization of the chaperones as described herein.

Molecular chaperones are any of a group of proteins that are involved in the correct intracellular folding and assembly of polypeptides without being components of the final structure. Molecular chaperones are found in bacteria, mitochondria, and the eukaryotic cytosol. Herein, “molecular chaperones” and “chaperones” are used interchangeably.

Herein, the term “mitochondriotropic” is used interchangeably with “mitochondrial targeting” and “mitochondrial-penetrating”.

Herein, the term “mitochondriotropic agent” refers to compositions having the formula A-B as described herein, wherein the agent inhibits chaperone activity and localizes to mitochondria.

As used herein, “Gamitrinib” refers to a geldanamycin analogue, e.g., 17-AAG, conjugated via an amino group at the C17 position via a linker to a mitochondrial penetrating moiety, for example, a tetraguanidinium (G4), triguanidinium (G3), diguanidinium (G2), monoguanidinium (G1), or a triphenylphosphonium (TPP) moiety. Throughout this application, the mitochondrial penetrating moiety that is part of a particular Gamitrinib is sometimes indicated. For example, Gamitrinib-G4 refers to a Gamitrinib in which a tetraguanidinium moiety is present. For example, Gamitrinib-TPP refers to a Gamitrinib in which a triphenylphosphonium moiety is present. Also throughout this application, the use of the plural form “Gamitrinibs” indicates one or more of the following: Gamitrinib-G4, Gamitrinib-G3, Gamitrinib-G2, Gamitrinib-G1, and Gamitrinib-TPP.

Although the following description is, at times, directed to the molecular chaperone Hsp90, it should be understood that the description can be generalized to structurally related molecular chaperones that are overexpressed in the mitochondria of cancer cells, e.g., TRAP-1 (Song et al., J. Biol. Chem. 270:3574-3581 (1995); Cechetto and Gupta, Experimental Cell Research 260:30-39 (2000)); Heat Shock 60 kDa Protein 1 (Hsp60/HspD1) (Singh et al., Biochem. Biophys. Res. Commun. 169 (2), 391-396 (1990)); Bross et al., J. Hum. Genet. 52 (1), 56-65 (2007)); and Heat shock 70 kDa protein 9 (HSPA9/mortalin) (Domanico et al., Mol. Cell. Biol. 13 (6), 3598-3610 (1993); Bhattacharyya et al., J. Biol. Chem. 270 (4), 1705-1710 (1995); Kaul et al., FEBS Lett. 361 (2-3), 269-272 (1995)).

By the term “enhancing” is meant an increase (e.g., a statistically significant increase) compared to a control. For example, the term “enhancing” can mean an induction of cancer to tumor cell death observed following therapeutic treatment compared to the amount of cancer or tumor cell death prior to the therapeutic treatment. In another example, the term “enhancing” can mean an increase in the rate or amount of cancer or tumor cell death observed following therapeutic treatment compared to the amount of cancer or tumor cell death in a control (e.g., a patient population not receiving the therapeutic treatment or in the same patient prior to administration of the therapeutic treatment).

By the term “autophagy inhibitor” is meant any molecule that decreases (e.g., a statistically significant decrease) autophagy in a cell (e.g., a cancer or tumor cell). Non-limiting examples of autophagy inhibitors are described herein and may be a protein, nucleic acid (e.g., antisense oligonucleotide, ribozyme, or siRNA), or a small molecule. Additional autophagy inhibitors are known in the art.

By the term “NF-κB signaling pathway inhibitor” is meant a molecule that decreases (e.g., a statistically significant decrease) one or more (e.g., two, three, or four) of the following in a cell (e.g., a tumor or cancer cell): the expression levels of NF-κB (protein or mRNA), activation of a cellular receptor tyrosine kinase that is upstream of NF-κB activation, activation of a cytosolic kinase that is upstream of NF-κB, phosphorylation and/or degradation of IκBα, nuclear translocation of NF-κB into the nucleus, NF-κB binding to κB promoter elements, and NF-κB transactivation of gene transcription.

By the term “NF-κB signaling pathway activity” is meant any detectable event in the NF-κB signaling pathway in a cell. For example, the term “NF-κB signaling pathway activity” includes the following molecular events: activation of a cellular receptor tyrosine kinase that is upstream of NF-κB activation, activation of a cytosolic kinase that is upstream of NF-κB, phosphorylation and/or degradation of IκBα, nuclear translocation of NF-κB into the nucleus, NF-κB binding to κB promoter elements, and NF-κB transactivation of gene transcription. Non-limiting methods for detecting NF-κB signaling pathway activity are described herein. Additional methods for detecting NF-κB signaling pathway activity are known in the art.

By the term “therapeutic-resistant cancer” is meant a cancer that does not respond or responds poorly to a cancer therapeutic. For example, a therapeutic-resistant cancer can show little or no response to a cancer therapeutic relative to the same type of cancer in another subject. A subject can be identified as having a therapeutic-resistant cancer by a health care professional (e.g., a physician, nurse, physician's assistant, or laboratory technician) following or prior to treatment with a cancer therapeutic. For example, a person can be identified as having a therapeutic-resistant cancer by measuring an increase (e.g., a statistically significant increase) in NF-κB signaling pathway activity in the cancer cells in the subject (e.g., either before or after administration of a cancer therapeutic). A person can be identified as having a therapeutic-resistant cancer by detecting little or no therapeutic effect following administration of a cancer therapeutic.

By the term “cancer cell resistant to a cancer therapeutic” is meant a cancer cell that does not undergo cell death (e.g., apoptosis) following administration of a cancer therapeutic. In some embodiments, a cancer cell is determined to be resistant to a cancer therapeutic when the cancer cell does not undergo cell death following administration of a cancer therapeutic, while cancer cell derived from a similar tissue in a control patient undergoes cell death following the administration of the same cancer therapeutic. In some embodiments, a cancer cell that is resistant to a cancer therapeutic is a cancer cell that has increased NF-κB signaling activity (e.g., either before or after administration of the cancer therapeutic).

The invention provides several advantages. For example, administration of both a mitochondrial-targeted chaperone inhibitor and a death receptor agonist, an autophagy inhibitor, or a NF-κB signaling pathway inhibitor can provide a strong additive and/or synergistic effect, as compared to administration of either individually. This effect was observed in tumor cells that are normally resistant to death receptor agonists when administered a mitochondrial-targeted chaperone inhibitor and a death receptor antagonist, but did not induce death in normal cells. In addition, a wide variety of tumor and cancer cell types show mitochondrial accumulation of chaperones and sensitivity to treatment with death receptor agonists, thus, numerous types of tumors and cancers can be treated by the methods described herein.

This invention provides mitochondriotropic agents (e.g., mitochondrially targeted chaperone inhibitors) that are advantageous over the art. These agents are localized to mitochondria with increased efficiency and selectively induce mitochondrial collapse and cell death in cells that show mitochondrial accumulation of chaperones (e.g., cancer cells). These agents have maximal effect on the function of mitochondrially-localized chaperones present in transformed cells (e.g., cancer cells) while having minimal effect on normal chaperone function (e.g., Hsp90 function in normal or non-transformed cells). Thus, these agents are ideal candidates for cancer therapy as they are expected to have lower toxicity than presently known chaperone inhibitors, which do not specifically inhibit mitochondrially-localized chaperones.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a bar graph depicting the effect of triphenylphosphonium geldanamycin (TPP-GA) and TRAIL, singly and in combination, on viability of U87, U251, and LN229 glioma cell lines and human fetal astrocyte (FHAS) cells.

FIG. 1B is a micrograph of LN229 cells treated with 500 ng/ml TRAIL, 5 μM TPP, or 500 ng/ml TRAIL+5 μM TPP and stained for viability.

FIGS. 2A-2D are scatter plots depicting flow cytometry analysis of U251 cells stained with Annexin V and propidium iodide (PI). 2A, control cells. 2B, cells treated with 25 ng/ml TRAIL. 2C, cells treated with 5 μM TPP-GA. 2D, cells treated with both 25 ng/ml TRAIL and 5 μM TPP-GA.

FIGS. 3A-3D are immunoblots of LN229 (3A, 3C) and U251 (3B, 3D) cells treated with the indicated concentrations of TRAIL (ng/ml) and TPP-GA (μg/ml) for the indicated times.

FIG. 4 is a timeline of experiments wherein xenografted mice were administered TPP and/or TRAIL.

FIGS. 5A and 5B are sets of luminescence images of mice before (5A) and after (5B) treatment with vehicle (PBS), TPP, TRAIL, or TRAIL+TPP. Relative luminescence for each set is shown on the accompanying legend.

FIGS. 6A and 6B are charts depicting observed luminescence before (6A) and after (6B) treatment with vehicle control, TPP, TRAIL, or TRAIL+TPP.

FIG. 7 is a chart depicting survival of xenografted mice treated with vehicle, TPP, TRAIL, or TRAIL+TPP.

FIGS. 8A and 8B are two electrophotomicrographs of an LN229 cell following treatment with G-TPP.

FIG. 8C is an immunophotomicrograph of an LN229 cell following treatment with G-TPP and staining with an antibody to COX-IV.

FIG. 9 is a set of five photomicrographs of LN229 cells left untreated (FIGS. 9A and 9B), incubated with non-targeted 17-AAG (10 μM; FIGS. 9C and 9D) for 16 hours, or treated with G-TPP and incubated with immuno-gold-conjugated, non-binding IgG (FIG. 9E). Scale bars are 2 μm (FIGS. 9A and 9C), 1 μm (FIGS. 9B and 9D), and 200 nm (FIG. 9E).

FIG. 10A is an immunoblot of lysates from U87 cells following treatment with G-TPP for the indicated periods of time. The position of the unmodified (I) and lipidated form (II) form of LC3 are indicated.

FIG. 10B is a fluorescence photomicrograph and a graph of the percentage of fluorescent U251 cells following transfection of U251 cells with LC3-GFP and treatment for 16 hours with vehicle (left photomicrograph) or G-TPP (right photomicrograph).

FIG. 11A is a graph of the percent viability of U87 cells following incubation with inhibitors of phagosome formation, bafilomycin A (BF) or 3-methyladenine (3-MA), for 1 hour and treatment with G-TPP. MTT analysis was performed after 16 hours with MTT. The mean data±standard deviation are shown.

FIG. 11B is an immunoblot of L229 cells following transfection with control or atg5-directed siRNA.

FIG. 11C is a graph showing the percent viability of L229 cells transfected with control or atg5-directed siRNA. MTT analysis was performed after 16 hours with MTT. The mean data±standard deviation are shown.

FIG. 12A is a graph showing the relative light units (RLU) of U251 cells from luciferase produced from a NF-κB promoter following no treatment or treatment with G-TPP or TRAIL. The luciferase data are normalized to β-galactosidase. TRAIL (800 ng/mL) was used as a control. The mean data±standard deviation are shown.

FIG. 12B is a graph showing the RLU of U251 cells from luciferase produced from a NF-κB promoter following no treatment or treatment with G-TPP in the presence or absence of TNF-α. The luciferase data are normalized to β-galactosidase. The mean data±standard deviation are shown.

FIG. 13A is a graph showing the RLU of U251 cells from luciferase produced from a FLIP promoter following no treatment or treatment with two different concentrations of G-TPP. The luciferase data are normalized to β-galactosidase. The mean data±standard deviation are shown.

FIG. 13B is two immunoblots lysate from LN229 cells following treatment with G-TPP. The top immunoblot is a dose response experiment. The bottom immunoblot is a time-course experiment. FLIP (L) is the FLIP long form. FLIP(S) is the FLIP short form.

FIG. 13C is a Northern blot showing the PCR-amplified expression of RelB, Bcl-3, and GAPDH mRNA in LN229 cells following treatment with G-TPP.

FIG. 14A is a graph showing the RLU of MCF-7 cells from luciferase produced from a p53 promoter following no treatment or treatment with G-TPP, Eto 10, Eto 10 and G-TPP, Eto 20, and Eto 20 and G-TPP. Cells were analyzed after 5 hours for β-galactosidase-normalized p53 luciferase promoter activity.

FIG. 14B is a graph showing the RLU of U251 cells from luciferase produced from a NF-κB or cFLIP promoter following treatment with non-targeted 17-AAG. Cells were analyzed after 9 hours for luciferase activity. The mean data±SEM of replicates of a representative experiment are shown.

FIG. 15A is an immunoblot showing the expression of CHOP and β-actin in LN229 cells following transfection with a control or CHOP-directed siRNA and treatment with G-TPP.

FIG. 15B is a graph showing the RLU of LN229 cells from luciferase produced from a NF-κB promoter following transfection with a control or CHOP-directed siRNA and treatment with G-TPP in the presence or absence of TNF-α. The mean data±standard deviation of replicates are shown. *P is 0.018 and ***P is 0.0007.

FIG. 16A is two immunoblots showing the expression of CHOP (top immunoblot) or C/EBPβ (bottom immunoblot) in LN229 cells following transfection with one of the indicated plasmids: pcDNA, CHOP, and C/EBPβ.

FIG. 16B is a graph of the RLU of LN229 cells from luciferase produced from a NF-κB promoter following transfection with pcDNA, C/EBPβ, or CHOP plasmids and treatment with TNF-α.

FIG. 17 is a graph of the RLU of LN229 cells from luciferase produced from a NF-κB promoter following transfection with pcDNA or a plasmid expressing a CHOP mutant lacking the leucine zipper (CHOP-ΔLeu) in the presence or absence of TNF-α. The mean data±standard deviation of replicates are shown. ***P is 0.0006.

### DETAILED DESCRIPTION

This application demonstrates that administration of mitochondrial-targeted chaperone inhibitors in combination with death receptor agonists, autophagy inhibitor, and/or NF-κB signaling pathway inhibitors can induce apoptosis in cancer cells, including glioblastoma, prostate, breast and colon cancer cells. Importantly, the concentrations of agents applied in the combination treatments only exhibited weak effects on cell death when administered as single treatments. Additionally, the combination of agents did not induce cell death in non-transformed cells. Without wishing to be bound by theory, the inhibition of mitochondrial chaperones may result in the sensitization of cells to death receptor agonist-, autophagy inhibitor, or NF-κB signaling pathway inhibitor-mediated cell death (e.g., apoptosis). In some embodiments, these combinations can be used to induce cell death and treat solid cancers. Also provided are methods of treating a therapeutic-resistant cancer in a subject by administering at least one mitochondrial-targeted chaperone inhibitor.

Thus, described herein are methods of enhancing apoptosis of cells and inducing cell death of tumor or cancer cells by administration of at least one death receptor agonist, autophagy inhibitor, and NF-κB signaling pathway inhibitor and at least one mitochondrial-targeted chaperone inhibitor. In some embodiments, the chaperone inhibitor is, e.g., an HSPA9, Hsp60, Hsp90, or TRAP-1 inhibitor. In some embodiments, the death receptor agonist is an agonist, e.g., a ligand, of tumor necrosis factor (TNF)-related apoptosis-inducing ligand receptor 1 (TRAILR1), TRAILR2, CD95, tumor necrosis factor receptor 1 (TNFR1), death receptor 3 (DR3), DR6, ectodysplasin A receptor (EDAR), or nerve growth factor receptor (NGFR). In some embodiments, the autophagy inhibitor is 3-methyladenine, bafilomycin A1, LY294002, wortmanin, hydroxychloroquine, chloroquine, 5-amino-4-imidazole carboxamide riboside, okadaic acid, a microcystin, microeystin, nodularin, analogues of cAMP, agents that elevate cAMP levels, adenosine, N6-mercaptopurine riboside, wortmannin, vinblastine, an antisense oligonucleotide, ribozyme, or siRNA that decreases the expression of MAP1LC3B, HSP90AA1, HSPA8, AMBRA1, ATG12, ATG16L1, ATG4A, ATG4B, ATG4C, ATG4D, ATG5, ATG9A, ATG9B, BECN1, GABARAP, GABARAPL1, GABARAPL2, IRGM, MAP1LC3A, RGS1, ULK1, ATG10, ATG16L1, ATG16L2, ATG3, ATG7, RAB24, DRAM, TMEM166, ATG3, AKT1, APP, ATG12, ATG5, BAD, BAK1, BAX, BCL2, BCL2L1, BID, BNIP3, CASP3, CASP8, CDKN1B, CDKN2A, CLN3, CTSB, CXCR4, DAPK1, DRAM, EIF2AK3, FADD, FAS, HDAC1, HTT, IFNA2, IFNG, IGF1, INS, MAPK8, NFKB1, PIK3CG, PRKAA1, PTEN, SNCA, SQSTM1, TGFB1, TGM2, TNF, TNFSF10, TP53, TP73, BAX, CDKN1B, CDKN2A, IFNG, PTEN, RB1, TGFB1, TP53, TP73, EIF2AK3, IFNA2, IFNA4, IFNG, ARSA, CTSS, EIF4G1, ESR1, GAA, HGS, MAPK14, PIK3C3, PIK3R4, PRKAA2, RPS6KB1, TMEM74, TMEM77, ULK2, and UVRAG. In some embodiments, the NF-κB signaling pathway inhibitor reduces IθBα phosphorylation and/or degradation, NF-κB nuclear translocation, NF-κB binding to a κB promoter element, and/or transactivation of transcription of an NF-κB target gene

These methods can be used to treat disorders associated with aberrant cellular proliferation, e.g., cancer and tumors, e.g., to kill cancer and tumor cells, e.g., in vivo and in vitro.

### I. Molecular Chaperones

Molecular chaperones, especially members of the Heat Shock Protein (Hsp) gene family (Lindquist and Craig, Annu. Rev. Genet. 1988; 22:631-77), assist in protein folding quality control, protein degradation, and protein trafficking among subcellular compartments (Hartl and Hayer-Hartl, Science 2002; 295:1852-8). This involves periodic cycles of ATPase activity, recruitment of additional chaperones, and compartmentalization in subcellular microdomains, including mitochondria (Young et al., Cell 2003; 112:41-50). Molecular chaperones have often been associated with enhanced cell survival (Beere, J. Cell Sci. 2004; 117:2641-51), via suppression of apoptosome-initiated mitochondrial cell death (Paul et al., Mol. Cell. Biol. 2002; 22:816-34), increased stability of survival effectors (Sato et al., Proc. Natl. Acad. Sci. U.S.A. 2000; 97:10832-37), and inactivation of p53 (Wadhwa et al., J. Biol. Chem. 1998; 273:29586-91). As described herein, the chaperone anti-apoptotic function play a central role in tumor cell maintenance and can be selectively targeted to kill cancer cells. See also: Whitesell et al., Nat. Rev. Cancer 2005; 5:761-72; and Isaacs et al., Cancer Cell 2003; 3:213-7.

The following is a brief description of some of the molecular chaperones that can be targeted using the present methods. In some embodiments, a molecular chaperone polypeptide useful in the present methods (e.g., in screening methods) is at least about 90%, 95%, 99%, or 100% identical to an amino acid sequence described herein (e.g., to a human sequence). In some embodiments, a nucleic acid encoding a molecular chaperone useful in the present methods (e.g., in screening methods) is at least about 90%, 95%, 99%, or 100% identical to a nucleic acid sequence described herein (e.g., to a human sequence).

The comparison of sequences and determination of percent identity between two sequences can be accomplished using a mathematical algorithm. For example, the percent identity between two amino acid sequences can determined using the Needleman and Wunsch, J. Mol. Biol. 1970, 48:444-453, algorithm which has been incorporated into the GAP program in the GCG software package (available on the world wide web at gcg.com), using the default parameters, e.g., a Blosum 62 scoring matrix with a gap penalty of 12, a gap extend penalty of 4, and a frameshift gap penalty of 5.

Hsp90 (Heat-Shock 90-kD Protein 1)

HSP90 is a molecular chaperone that plays a key role in the conformational maturation of a number of proteins, including oncogenic signaling proteins. As described herein, Hsp90 accumulates in the mitochondria of cancer cells, but not normal cells, and can be targeted using the compositions described herein including a mitochondrial-penetrating sequence.

GenBank Acc. Nos. for human Hsp90 include NM_{—}001017963.2 (nucleic acid) and NP_{—}001017963.2 (protein), for heat shock protein 90 kDa alpha (cytosolic), class A member 1 isoform 1, and NM_{—}005348.3 (nucleic acid) NP_{—}005339.3 (protein), for heat shock protein 90 kDa alpha (cytosolic), class A member 1 isoform 2. Variant 2 differs in the 5′ UTR and coding sequence compared to variant 1. The resulting isoform 2 is shorter at the N-terminus compared to isoform 1.

Hsp90 is also known as HSPCA; HSPC1; HSP90A; HSP89-ALPHA (HSP89A); Lipopolysaccharide-Associated Protein 2 (LAP2); and LPS-associated protein 2.

TRAP-1 (TNF Receptor-Associated Protein 1)

TRAP-1 has high homology to hsp90, and binds the type 1 tumor necrosis factor receptor (see Song et al., J. Biol. Chem. 1995, 270:3574-81). The deduced 661-amino acid protein is 60% similar to HSP90 family members, although it lacks the highly charged domain found in HSP90 proteins. See, e.g., Felts et al., J. Biol. Chem. 2000; 275:3305-12. As described herein, TRAP-1 accumulates in the mitochondria of cancer cells, but not normal cells, and can be targeted using the compositions described herein including a mitochondrial-penetrating sequence.

GenBank Acc. Nos. for human TRAP-1 include NM_{—}016292.2 (nucleic acid) and NP_{—}057376.2 (amino acid). TRAP-1 is also referred to as Heat-Shock Protein, 75-KD (HSP75); Tumor Necrosis Factor Receptor-Associated Protein 1; TRAP1; and TNFR-Associated Protein 1.

Hsp60 (Heat-Shock 60-kD Protein 1)

Hsp60, together with its associated chaperonin, Hsp10, has been recognized as an evolutionary conserved stress response chaperone (Zhao et al., EMBO J. 2002; 21:4411-19), largely, but not exclusively compartmentalized in mitochondria (Soltys and Gupta, Int. Rev. Cytol. 2000; 194:133-196), and with critical roles in organelle biogenesis and folding/refolding of imported preproteins (Deocaris et al., Cell Stress Chaperones 2006; 11:116-128). However, whether Hsp60 also contributes to cell survival is controversial, with data suggesting a pro-apoptotic function via enhanced caspase activation (Samali et al., EMBO J. 1999; 18:2040-8; Xanthoudakis et al., EMBO J. 1999; 18:2049-56), or, conversely, an anti-apoptotic mechanism involving sequestration of Bax-containing complexes (Shan et al., J. Mol. Cell. Cardiol. 2003; 35:1135-43). A role of Hsp60 in cancer was equally uncertain, as up- (Thomas et al., Leuk. Res. 2005; 29:1049-58; Cappello et al., BMC Cancer 2005; 5:139), or down-regulation (Tang et al., Cell Stress Chaperones 2005; 10:46-58; Cappello et al., Cancer 2006; 107:2417-24) of this chaperone has been reported in various tumor series correlating with disease outcome. As described herein, Hsp60 is highly expressed in tumor cells, as compared to normal cells, and targeting of Hsp60 causes mitochondrial dysfunction and apoptosis, whereas loss of Hsp60 in normal cells is well tolerated, and does not result in cell death.

Hsp60 is also known as CPN60; GROEL; HSP60; HSP65; SPG13; and HuCHA60. Exemplary GenBank Acc. Nos. for human Hsp60 include NM_{—}002156.4 (nucleic acid) and NP_{—}002147.2 (protein) for transcript variant 1 (the longer variant), and NM_{—}199440.1 (nucleic acid) and NP_{—}955472.1 (protein) for transcript variant 2. Variant 2 differs in the 5′ UTR compared to variant 1. Both variants 1 and 2 encode the same isoform.

HspA9 (Heat Shock 70 kDa Protein 9

HspA9 belongs to the heat shock protein 70 family, which contains both heat-inducible and constitutively expressed members. The latter are called heat-shock cognate proteins, of which HspA9 is one. HspA9 plays a role in the control of cell proliferation, and may also act as a chaperone. See, e.g., Wadhwa et al., Int. J. Cancer 2006; 118:2973-80; Wadhwa et al., J. Gene Med. 2004; 6:439-44.

HspA9 is also known as mortalin, mthsp70, and GRP75. Exemplary GenBank Acc. Nos. for human HspA9 include NM_{—}004134.5 (nucleic acid) and NP_{—}004125.3 (protein), the heat shock 70 kDa protein 9 precursor.

### II. Inhibitors of Molecular Chaperones

The compositions and methods described herein include the use of inhibitors of molecular chaperones, e.g., inhibitors or Hsp60, HspA9, Hsp90 and/or TRAP-1. The inhibitors useful in the methods and compositions described herein act directly on the chaperone protein itself, i.e., they do not act upstream or downstream. A number of such inhibitors are known in the art, e.g., peptide inhibitors and small molecule inhibitors. In some embodiments, the molecular chaperone inhibitors useful in this invention inhibit the ATPase activity of the chaperone, e.g., of Hsp60, HspA9, Hsp90, and/or TRAP-1. In some embodiments, the molecular chaperone inhibitors useful in this invention inhibit the binding of Hsp60, HspA9, Hsp90, or TRAP-1 to Cyclophilin D. In some embodiments, the molecular chaperone inhibitors useful in this invention inhibit the binding of Hsp60, HspA9, Hsp90, or TRAP-1 to survivin. In some embodiments, molecular chaperone inhibitors bind to a chaperone, and induce the proteasomal degradation of the chaperone's client proteins. Exemplary molecular chaperone inhibitors are described in US 2009/0099080, herein incorporated by reference in its entirety.

In addition, there are numerous methods useful for identifying, designing, and assaying candidate chaperone inhibitors. For example, rational screening methods have been used to identify additional molecules that target Hsp90, using a computational approach using a shepherdin peptide (LFACGSSHK, all D-amino acids, as a scaffold to screen a database of nonpeptidic structures. See, e.g., Meli et al., J. Med. Chem. 49:7721-7730 (2006).

Peptide Inhibitors of Molecular Chaperones

A number of peptide inhibitors of molecular chaperones, e.g., of Hsp90 and/or TRAP-1, are known in the art. The inhibitors useful in the compositions and methods described herein can include the entire peptide or polypeptide (e.g., all of an apoptosis-inducing protein (AIP such as survivin), or an active (i.e., inhibitory) fragment thereof that retains the Hsp90 inhibitory activity of the parent, i.e., at least 40% of the activity of the parent; an active fragment preferably has at least 50%, 60%, 70%, 80%, 90%, 100% or more of the Hsp90-inhibitory activity of the parent polypeptide.

Survivin Peptides and Derivatives

Survivin peptides and peptide derivatives are disclosed in US 2006/0035837, herein incorporated by reference in its entirety. Active survivin peptides share a core Hsp90 binding sequence motif of SEQ ID NO:2 (His Ser Ser Gly Cys), which is located in the single Baculovirus Inhibitor of Apoptosis (IAP) Repeat (BIR) domain of the Survivin protein. This motif corresponds to amino acid residues at position 80-84 of full-length Survivin (SEQ ID NO:1). Peptides including this motif, and peptide derivatives thereof, can (a) bind to the N-terminal ATPase domain of Hsp90 (the “ATP pocket”) and (b) inhibit Hsp90-Survivin protein-protein interactions in vitro and in vivo.

The terms Survivin peptide and Survivin peptide derivative, as used herein, refer to peptides that include less than the complete amino acid sequence of a functional Survivin protein that prevents cell death. Survivin peptides and peptide derivatives useful to this invention inhibit molecular chaperones and in particular, inhibit interaction between a molecular chaperone, e.g., Hsp90 or TRAP-1, and Cyclophilin D.

The full-length human, wild type Survivin polypeptide has the following amino acid sequence:

(SEQ ID NO: 1)
MGAPTLPPAWQPFLKDHRISTFKNWPFLEGCACTPERMAEAGFIHCPT
ENEPDLAQCFFCFKELEGWEPDDDPIEEHKKHSSGCAFLSVKKQFEEL
TLGEFLKLDRERAKNKIAKETNNKKKEFEETAKKVRRAIEQLAAMD

The following table (Table 1) lists some exemplary Survivin peptides that can bind to Hsp90:

TABLE 1
Exemplary Survivin peptides

| | |
|---|---|
| SEQ ID NO: 2 | His Ser Ser Gly Cys |
| SEQ ID NO: 3 | Lys His Ser Ser Gly Cys Ala Phe Leu |
| | Ser Val Lys |
| SEQ ID NO: 4 | Ile Asp Asp His Lys Lys His Ser Ser |
| | Gly Cys Ala Phe Leu |
| SEQ ID NO: 5 | Lys Lys His Ser Ser Gly Cys Ala Phe |
| | Leu |
| SEQ ID NO: 6 | Lys His Ser Ser Gly Cys |
| SEQ ID NO: 7 | His Ser Ser Gly Cys Ala |
| SEQ ID NO: 8 | Lys His Ser Ser Gly Cys Ala |
| SEQ ID NO: 9 | Lys Lys His Ser Ser Gly Cys |
| SEQ ID NO: 10 | His Ser Ser Gly Cys Ala Phe |
| SEQ ID NO: 11 | His Lys Lys His Ser Ser Gly Cys Ala |
| | Phe Leu Ser Val Lys Lys |
| SEQ ID NO: 12 | Lys His Ser Ser Gly Cys Ala Phe Leu |

Variants of Survivin peptides can also be used in the methods and compositions described herein. Conservative and non-conservative amino acid substitutions may be made. In particular, conservative amino acid substitutions can be made for one or more, e.g., up to five, ten, twenty, or thirty, amino acids outside of the core pentamer sequence corresponding to His 80 to Cys 84 in SEQ ID NO:1 (i.e., SEQ ID NO:2 set forth above). Peptidomimetics of Survivin peptides are described by Plescia et al., Cancer Cell 2005, 7:457-68, and US 2006/0035837, both incorporated herein by reference in their entirety.

Other IAP Peptides and Derivatives

Other Inhibitors of Apoptosis Proteins (IAPs) interact with Hsp90, including cIAP1 (Entrez Accession No.: NP_{—}001156.1), cIAP2 (Entrez Accession No.: NP_{—}001157.1), and XIAP (Entrez Accession No.: NP_{—}001158.2). See, e.g., Deveraux and Reed, Genes Dev. 1999, 13:239-252. These IAP proteins contain at least one Baculovirus IAP repeat domain that mediates Hsp90 interactions, as disclosed herein. For example, the first BIR domain of XIAP (BIR1) mediates Hsp90-XIAP binding interactions.

IAP proteins, or Hsp90-binding and -inhibiting fragments thereof, can therefore be used in the present compositions and methods. For example, peptides corresponding to one or more BIR domains in these IAP proteins, or Hsp90-binding fragments thereof, can be used in the compositions and methods disclosed herein to induce cancer or tumor cell death. IAP proteins, or Hsp90-binding fragments thereof, can also be screened as test compounds, e.g., to identify candidate compounds that inhibit binding between molecular chaperones and Cyclophilin D. In some embodiments, IAP proteins, or Hsp90-binding fragments thereof, can be screened as test compounds to identify candidate compounds that induce cancer cell death.

An exemplary first BIR domain of XIAP includes the sequence:

(SEQ ID NO: 14)
RLKTFANFPSGSPVSASTLARAGFLYTGEGDTVRCFSCHAAVDRWQYG
DSAVGRHRKVSPNCRFIN

An exemplary first BIR domain of cIAP1 includes the sequence:

(SEQ ID NO: 15)
RMSTYSTFPAGVPVSERSLARAGFYYTGVNDKVKCFCCGLMLDNWKRG
DSPTEKHKKLYPSCRFVQ

An exemplary first BIR domain of cIAP2 includes the sequence:

(SEQ ID NO: 16)
RMSTYSTFPAGVPVSERSLARAGFYYTGVNDKVKCFCCGLMLDNWKLG
DSPIQKHKQLYPSCSFIQ

Variants of Peptide Inhibitors

Variants of peptide inhibitors of molecular chaperones are also part of this invention. These include sequence variants. Where a conservative amino acid substitution is made, the substitution can be of one amino acid residue for another in any of the following groups: arginine, histidine, and lysine; aspartic acid and glutamic acid; alanine, leucine, isoleucine and valine; and phenylalanine, tryptophan and tyrosine. The amino acid residues listed here are naturally occurring. Non-naturally occurring amino acid residues of like kind may also be substituted. For example, a negatively charged non-naturally occurring amino acid residue may be substituted for a negatively charged naturally occurring amino acid residue; a hydrophobic aromatic non-naturally occurring amino acid residue may be substituted for a hydrophobic aromatic naturally occurring amino acid residue; and so forth.

The degree of identity can vary and can be determined by methods well established in the art. “Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. A biologically active variant of a polypeptide described herein can have at least or about 80%, 85%, 90%, 95%, 96%, 97%, 98%, or 99% identity or homology to a corresponding naturally occurring polypeptide (e.g., a survivin fragment or a IAP fragment, e.g., as described herein). The nucleic acids encoding the biologically active variant polypeptides can be similarly described as having at least or about 80%, 85%, 90%, 95%, 96%, 97%, 98%, or 99% identity to a corresponding naturally occurring nucleic acid sequence. Those of ordinary skill in the art will readily recognize degenerate variants of nucleic acid sequences, and such variants can be used for the purposes described herein.

When using a peptide inhibitor and/or mitochondrial penetrating moiety in a human subject, it will generally be desirable to use a human or humanized sequence. Thus, the methods described herein can include using standard molecular biology techniques to humanize a non-human sequence. Alternatively, human sequences can be used to make the construct.

Modifications of Peptide Inhibitors

Modified versions of the peptides described herein can also be used in the compositions and methods described herein. The peptides and biologically active variants thereof can be modified in numerous ways. For example, agents, including additional amino acid residues, other substituents, and protecting groups can be added to either the amino terminus, the carboxy terminus, or both. The modification can be made for the purpose of altering the peptides' form or altering the way the peptides bind to or interact with one another, with non-identical peptides, or with other polypeptides. For example, the peptides can be modified to include cysteine residues or other sulphur-containing residues or agents that can participate in disulphide bond formation. For example, one can add at least two cysteine residues, one or both of which are, optionally, at the C-terminal or N-terminal of the peptide.

The peptides can be cyclized by formation of a disulfide bond between cysteine residues (or, more generally, between two of the at least two cysteine residues present in the polypeptide (e.g., at the terminal regions)). While the peptides of the present invention may be linear or cyclic, cyclic peptides generally have an advantage over linear peptides in that their cyclic structure is more rigid and hence their biological activity may be higher than that of the corresponding linear peptide (see, generally, Camarero and Muir, J. Am. Chem. Soc. 1999, 121:5597-98.

Strategies for the preparation of circular polypeptides from linear precursors have been described and can be employed with the present peptides. For example, a chemical cross-linking approach can be used to prepare a backbone cyclized version of the peptide (Goldenburg and Creighton, J. Mol. Biol. 1983, 165:407-413). Other approaches include chemical intramolecular ligation methods (see, e.g., Camarero et al., Angew Chem. Int. Ed. 1998, 37:347-349; Tam and Lu, Prot. Sci. 1998, 7:1583-92; Camarero and Muir, Chem. Commun. 1997, 1369-70; and Zhang and Tam, J. Am. Chem. Soc. 1997, 119:2363-70) and enzymatic intramolecular ligation methods (Jackson et al., J. Am. Chem. Soc. 1995, 117:819-820), which allow linear synthetic peptides to be efficiently cyclized under aqueous conditions. See also U.S. Pat. No. 7,105,341.

Alternatively, or in addition, the peptide can further include a substituent at the amino-terminus or carboxy-terminus. The substituent can be an acyl group or a substituted or unsubstituted amine group (e.g., the substituent at the N-terminus can be an acyl group and the C-terminus can be amidated with a substituted or unsubstituted amine group (e.g., an amino group having one, two, or three substituents, which may be the same or different)). The amine group can include a lower alkyl (e.g., an alkyl having 1-4 carbons), alkenyl, alkynyl, or haloalkyl group. The acyl group can be a lower acyl group (e.g., an acyl group having up to four carbon atoms), especially an acetyl group.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, and the like.

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to aromatic monocyclic or multicyclic groups containing from 6 to 19 carbon atoms. Examples of aryl groups include, but are not limited to unsubstituted or substituted phenyl, unsubstituted or substituted fluorenyl, and unsubstituted or substituted naphthyl.

As used herein, “heterocycloalkyl” refers to a monocyclic or multicyclic, saturated or unsaturated ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In certain embodiments, one of the atoms of the ring can be replaced with a carbonyl or sulfonyl group.

As used herein, “alkylene,” “alkenylene,” “alkynylene,” “cycloalkylene,” “arylene,” “heteroarylene,” and “heterocycloalkylene” refer to divalent linking “alkyl,” “alkenyl,” “alkynyl,” “cycloalkyl,” “aryl,” “heteroaryl,” and “heterocycloalkyl” groups. The divalent linkers, in some embodiments, can be present in both directions, e.g., a C(O)NH can either be —C(O)NH— or —NHC(O)—.

As noted, the peptides can vary in length and can be or can include contiguous amino acid residues that naturally occur in chaperone binding proteins (CBP), e.g., Survivin or IAPs, or that vary to a certain degree from naturally occurring CBP sequences (but retain sufficient activity to be useful). Where the peptides include, at their N-terminus or C-terminus (or both), amino acid residues that are not naturally found in CBPs, the additional sequence(s) can be about 200 amino acid residues long, and these residues can be divided evenly or unevenly between the N- and C-termini. For example, both the N- and C-termini can include about 10, 20, 30, 40, 50, 60, 70, 80, 90, or 100 amino acid residues. Alternatively, one terminus can include about 10, 20, 30, 40, 50, 60, 70, 80, 90, 100, 110, 120, 130, 140, 150, 160, 170, 180, 190, or 200 residues, and one terminus can include none (e.g., it can terminate in an amino acid sequence identical to a naturally occurring Survivin sequence).

More specifically, the N- or C-termini can include 1 to about 100 (e.g., 1, 2, 3, 4, 5, 6, 7, 8, 9, 10, 15, 20, 25, 30, 40, 50, 60, 70, 80, 90, or 100) amino acid residues that are positively charged (e.g., basic amino acid residues such as arginine, histidine, and/or lysine residues); 1 to about 100 amino acid residues that are negatively charged (e.g., acidic amino acid residues such as aspartic acid or glutamic acid residues); 1 to about 100 glycine residues; 1 to about 100 hydrophobic amino acid residues (e.g., hydrophobic aliphatic residues such as alanine, leucine, isoleucine or valine or hydrophobic aromatic residues such as phenylalanine, tryptophan or tyrosine); or 1 to about 100 (e.g., 1-4) cysteine residues.

The peptides, including the modified peptides described above, can be protease resistant and can include one or more types of protecting groups such as an acyl group, an amide group, a benzyl or benzoyl group, or a polyethylene glycol. More specifically, a peptide, including the modified peptides described above, can be N-terminally acetylated and/or C-terminally amidated.

Where non-naturally occurring or modified amino acid residues are included they can be selected from the following or many others available in the art: 4-hydroxyproline, gamma-carboxyglutamic acid, o-phosphoserine, o-phosphotyrosine, or delta-hydroxylysine. Other examples include naphthylalanine, which can be substituted for trytophan to facilitate synthesis, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl. Peptides having non-naturally occurring amino acid residues may be referred to as synthetic peptides and constitute one type of variant as described herein. Other variants include peptides in which a naturally occurring side chain of an amino acid residue (in either the L- or D-form) is replaced with a non-naturally occurring side chain.

In one embodiment, the peptides can have three extra amino acids (Met-Gly-Ser) at either terminus (or both) (e.g., at the N-terminus) and seven to eight extra amino acids (Thr-Ser-His-His-His-His-His-His-Cys (SEQ ID NO:13)) at either terminus (or both) (e.g., at the C-terminus).

In another embodiment, the peptides can be PEGylated by methods known in the art.

For guidance on peptide modification by reduction/alkylation and/or acylation, one can consult Tarr, Methods of Protein Microcharacterization, J. E. Silver ed., Humana Press, Clifton N.J. (1986) 155-194; for guidance on chemical coupling to an appropriate carrier, one can consult Mishell and Shiigi, eds, Selected Methods in Cellular Immunology, WH Freeman, San Francisco, Calif. (1980) and U.S. Pat. No. 4,939,239; and for guidance on mild formalin treatment, one can consult Marsh, Int. Arch. Allergy Appl. Immunol. (1971) 41:199-215.

Peptidomimetics of the inhibitory peptides can also be used. Peptide inhibitors disclosed herein and known in the art can be modified according to methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem. 278:45746, (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as ∀-amino phosphonic acids and ∀-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. Exemplary Survivin retro-inverso peptidomimetics include LFACGSSHK (SEQ ID NO:25), CGSSH (SEQ ID NO:26), GSSHK (SEQ ID NO:27), KKWKMRRNQFWVKVQRLFACGSSHK (SEQ ID NO:28), KKWKMRRNQFWVKVQRCGSSH (SEQ ID NO:29), and KKWKMRRNQFWVKVQRGSSHK (SEQ ID NO:30), wherein the sequences include all D-amino acids. These sequences can be modified, e.g., by biotinylation of the amino terminus and amidation of the carboxy terminus.

Any of the peptides described herein, including the variant forms described herein, can further include a heterologous polypeptide (e.g., a polypeptide having a sequence that does not appear in a CBP). The heterologous polypeptide can be a polypeptide that increases the circulating half-life of the peptide to which it is attached (e.g., fused, as in a fusion protein). The heterologous polypeptide can be an albumin (e.g., a human serum albumin or a portion thereof) or a portion of an immunoglobulin (e.g., the Fc region of an IgG). The heterologous polypeptide can be a mitochondrial-penetrating moiety.

Compounds mimicking the necessary conformation of the peptides described herein are contemplated as within the scope of this invention. A variety of designs for such mimetics are possible. U.S. Pat. No. 5,192,746; U.S. Pat. No. 5,169,862; U.S. Pat. No. 5,539,085; U.S. Pat. No. 5,576,423; U.S. Pat. No. 5,051,448; and U.S. Pat. No. 5,559,103, all hereby incorporated by reference, describe multiple methods for creating such compounds.

Non-peptidic compounds that mimic peptide sequences are known in the art (Meli et al., J. Med. Chem. 2006, 49:7721-30, describing methods of identifying nonpeptide small molecule mimics of shepherdin). Synthesis of non-peptide compounds that mimic peptide sequences is also known in the art (see, e.g., Eldred et al. J. Med. Chem. 1994, 37:3882; Ku et al. J. Med. Chem. 1995, 38:9; Meli et al., J. Med. Chem. 2006, 49:7721-30). Such nonpeptide compounds that mimic CBP peptides that bind chaperones are specifically contemplated by the present invention.

The present invention also contemplates synthetic mimicking compounds. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the (α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to the herein-described chaperone inhibitor peptides. These mimetics include, but are not limited to, peptides in which L-amino acids are replaced by their D-isomers. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant (Pearson and Lipman, Proc. Natl. Acad. Sci. U.S.A. 1988, 85:2444-48; Lipman and Pearson, Science 1985, 227:1435-41). More generally, the CBP peptides described herein and the mimetics described above can be synthesized using any known methods, including tea-bag methodology or solid phase peptide synthesis procedures described by Merrifield et al., Biochemistry 1982, 21:5020-31; Houghten Wellings, Proc. Natl. Acad. Sci. U.S.A. 1985, 82:5131135; Atherton, Meth. Enzymol. 1997, 289:44-66; or Guy and Fields, Meth. Enzymol. 1997, 289:67-83, or using a commercially available automated synthesizer.

Small Molecule Inhibitors of Molecular Chaperones

A number of small molecule chaperone inhibitors useful in the methods and compositions described herein are known in the art. For example, small molecule chaperone inhibitors that are useful in the compositions and methods described herein include, but are not limited to, molecules that bind to a Hsp90 ATP binding pocket. Small molecule Hsp90 inhibitors known in the art are described, for example, in Rodina et al., Nat. Chem. Biol. 2007, 3:498-507.

In some embodiments, the chaperone inhibitor is an Hsp90 inhibitor selected from one of several chemotypes. Two of these chemotypes are ansamycin and macrolactone inhibitors. These are represented by radicicol and cycloproparadicicol, members of the macrolactone Hsp90 inhibitor class, and 17-dimethylaminoethylamino-17-demethoxy-geldanamycin (17DMAG) and 17AAG, members of the ansamycin class of Hsp90 inhibitors. The structural basis for inhibition of Hsp90 by radicicol and geldanamycin is known, so one of skill in the art could readily generate and test analogs thereof that would retain Hsp90 inhibitory activity, see, e.g., Roe et al., J. Med. Chem. 1999, 42:260-266. Purine inhibitors form a third class of compounds useful in the compositions and methods described herein.

Ansamycin Inhibitors of Hsp90

Further examples of molecular chaperone inhibitors that are useful in this invention include, but are not limited to, quinine ansamycin antibiotics, such as the macbecins, geldanamycin, geldanamycin analogues, and herbimycin A.

Geldanamycin is an inhibitor of heat shock protein-90 (Hsp90), which is involved in the folding, activation and assembly of a wide range of proteins (“client proteins”), including key proteins involved in signal transduction, cell cycle control and transcriptional regulation. The binding of geldanamycin to Hsp90 disrupts Hsp90-client protein interactions, preventing the client proteins from folding correctly. Geldanamycin and geldanamycin analogues are part of this.

As used herein, “geldanamycin analogues” refers to compounds that share a common core structure with geldanamycin but have minor chemical modifications.

Geldanamycin analogues that are variant at position 17 of geldanamycin are known in the art and many are commercially available. Examples of commercially available geldanamycin analogues include, but are not limited to, 17-allylamino-demethoxygeldamycin (17-AAG), 17-dimethylaminogeldanamycin, 17-GMB-APA-GA (a maleimido derivative of geldanamycin that enables the conjugation of GA to a polypeptide), 17-(Dimethylaminoethylamino)-17-demethoxygeldanamycin (17-DMAG), 17-[2-(Pyrrolidin-1-yl)ethyl]amino-17-demethoxygeldanamycin (17-AEP-GA), and 17-(Dimethylaminopropylamino)-17-demethoxygeldanamycin (17-DMAP-GA). See also Sasaki et al., U.S. Pat. No. 4,261,989; Schnur et al., U.S. Pat. No. 5,932,566; Schnur et al., J. Med. Chem. 1995, 38:3806-12; Schnur et al., J. Med. Chem. 1995, 38:3813-20; and Santi et al., US 2003/0114450.

Geldanamycin analogues that are variant at position 11 are known in the art. Examples include, but are not limited to, Muroi et al., U.S. Pat. No. 4,421,688; Schnur, U.S. Pat. No. 5,387,584; Schnur et al., U.S. Pat. No. 5,932,566; Welch et al., U.S. Pat. No. 6,015,659; Whitesell et al., WO 94/08578; Ho et al., WO 00/03737; Snader et al., WO 02/36574; Snader et al., WO 02/079167; Santi et al., WO 03/013430; Zhang et al., WO 03/066005; Omura et al., JP 63-218620; Schnur et al., J. Med. Chem. 1995, 38:3806-12; and Schnur et al., J. Med. Chem. 1995, 38:3813-20; which are herein incorporated by reference. 11-O-methylgeldanamycin compounds known in the art are described in U.S. Pat. No. 6,855,705, U.S. Pat. No. 6,887,993, and U.S. Pat. No. 6,870,049.

In some embodiments of the composition, the molecular chaperone inhibitor includes geldanamycin analogues:

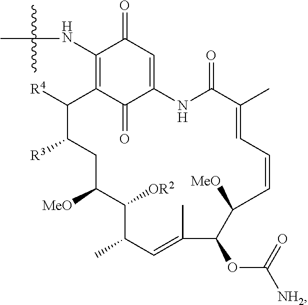

where, R² is H, alkyl, aryl, or arylalkyl; R³ is H, alkyl; and R⁴ is H, alkyl, alkenyl, aryl, arylalkyl, OR^{d}, wherein R^{d} is H, alkyl, or arylalkyl.

In some embodiments of the composition, R² is H or alkyl; R³ is H, alkyl; and R⁴ is H, or OR^{d}, wherein R^{d} is H, alkyl.

In some embodiments of the composition, R² is H; R³ is methyl; and R⁴ is H.

Resorcinol-Derived Inhibitors of Hsp90

Compounds derived from Resorcinol are potent inhibitors of Hsp90. These include compounds based on the 4,5-diarylisoxazole scaffold (see, for example, Brough et al., J. Med. Chem. 2008, 51:196-218), compounds based on the 3,4-diarylpyrazole scaffold (see, for example, U.S. Pat. No. 7,247,734 and Sharp et al., Cancer Res. 2007, 67:2206-16), and 3,4-diaryl pyrazole resorcinol HSP90 inhibitor (CCT018159), amide resorcinol compounds (as described, for example, in WO 2006/117669), and isoxazole resorcinol compounds. See also Sharp et al., Mol. Cancer. Ther. 2007, 6:1198-1211 (synthetic, potent resorcinylic pyrazole/isoxazole amide analogues, e.g., VER-49009 and the corresponding isoxazole VER-50589); Eccles et al., Cancer Res. 2008, 68:2850-60 (NVP-AUY922, a novel resorcinylic isoxazole amide heat shock protein 90 (HSP90) inhibitor); Barril et al., Bioorg. Med. Chem. Lett. 2006, 16:2543-48 (piperazinyl, morpholino and piperidyl derivatives of the pyrazole-based Hsp90 inhibitor CCT018159).

Macrolactone-Hsp90 Inhibitors

The macrocyclics radicicol and monocillin, and analogs thereof such as cycloproparadicicol, are inhibitors that bind the ATP-binding site of Hsp90. See, e.g., Turbyville et al., J. Nat. Prod. 2006, 69:178-184; Soga et al., Curr. Cancer Drug Targets 2003, 3:359-69; Shiotsu et al., Blood 2000, 96:2284-91; and U.S. Pat. No. 7,115,651. KF25706, a novel oxime derivative of radicicol, has in vivo antitumor activity via selective depletion of Hsp90 binding signaling molecules (Soga et al., Cancer Res. 1999, 59:2931-38).

Chimeric inhibitors that include structural components of radicicol and geldanamycin are also known, see, e.g., Hadden et al., Curr. Top. Med. Chem. 6:1173-82; Shen et al., J. Org. Chem. 2006, 71:7618-31.

Purine Inhibitors of Hsp90

Hsp90 inhibitors of the purine-scaffold class have been reported to be potent and selective against Hsp90 both in vitro and in vivo models of cancer, and the structural basis of this activity has been determined. See Wright et al., Chem. Biol. 2004, 11:775-85. Several 8-Aryl-Sulfanyl Adenine compounds have been synthesized and shown to have Hsp90 inhibitory activity, e.g., PU-H71 and PU-H64, the structures of which have been solved with Hsp90. See Immormino et al., J. Med. Chem. 2006, 49:4953-60. Other purine class Hsp90 inhibitors are known in the art and include, for example, 3,4-diaryl pyrazoles and related analogs (McDonald et al., Curr. Top. Med. Chem. 2006, 6:1193-1203); pyrazolopyrimidines and related analogs (U.S. Pat. No. 7,148,228), pyrrolopyrimidines and related analogs (U.S. Pat. No. 7,138,402), and 2-aminopurine analogs (U.S. Pat. No. 7,138,401).

Hsp60 Inhibitors

Several Hsp60 inhibitors are known in the art, including epolactaene (Nagumo et al., Biochem. J. 2005, 387:835-840; Tan and Negishi, Org. Lett. 2006, 8:2783-85; and Nagumo et al., Bioorg. Med, Chem. Lett. 2004, 14:4425-29), and mizoribine (bredinin) (Itoh et al., J. Biol. Chem. 1999, 274:35147-51).

HspA9 (Mortalin) Inhibitors

MKT-077, a cationic rhodacyanine dye analogue with selective toxicity to cancer cells, binds to HspA9/mortalin, and abrogates its interactions with the tumor suppressor protein, p53. See, e.g., Wadhwa et al., Cancer Res. 2000; 60:6818-21.

Other Inhibitors

Molecular chaperone inhibitors that are useful in this invention also include molecules that inhibit interaction between Hsp60 and Cyclophilin D, Hsp90 and Cyclophilin D, or TRAP-1 and Cyclophilin D. These inhibitors may be identified from molecules known in the art, or present in chemical libraries by the methods described herein; see, e.g., Meli et al., J. Med. Chem. 2006, 49:7721-30, and Howes et al., Anal. Biochem. 2006, 350:202-213. For example, the non-peptidic small molecule 5-aminoimidazole-4-carboxamide-1-beta-D-ribofuranoside (AICAR) was identified as a structurally novel inhibitor of Hsp90 (see Meli et al., 2006, supra), and can be used in the methods described herein. See also Blagg et al., Med. Res. Rev. 2005, 26:310-338.

### III. Mitochondrial-Penetrating Moieties

Described herein are mitochondrial penetrating molecular chaperone inhibitors. Any of the molecular chaperone inhibitors described herein can be modified by association with mitochondrial-penetrating moieties using methods known in the art, with the proviso that if the chaperone inhibitor is Shepherdin or an active fragment thereof, the mitochondrial-penetrating moiety is not Antennapedia or a fragment thereof. Examples are given below.

As used herein, a mitochondrial-penetrating moiety is a chemical group, e.g., a peptide, peptidomimetic, or other compound, that increases mitochondrial localization of an associated, e.g., chemically conjugated, molecular chaperone inhibitor, as compared to the molecular chaperone inhibitor alone.

Peptide Mitochondrial-Penetrating Moieties In the compositions described herein, a chaperone inhibitor (as described herein) can be attached to a peptide mitochondrial-penetrating moiety. For example, an Antennapedia carrier sequence, corresponding to a sequence found on the third α-helix of the Antennapedia (Gratton et al., Cancer Cell 2003, 4:31), can be used. An exemplary sequence of such a peptide is RQIKIWFQNRRMKWKK (SEQ ID NO:18), herein ANT. Other examples of targeting peptides to which the chaperone inhibitors disclosed herein can be attached include, but are not limited to, e.g., the TAT protein sequence from HIV-1 (Chen et al., Proc. Natl. Acad. Sci. U.S.A. 1999, 96:4325; Kelemen et al., J. Biol. Chem. 2002, 277:8741-48), e.g., RKKRRQRRR (SEQ ID NO:19) (Brooks et al., Adv. Drug Del. Rev. 2005, 57:559-577), or a modified TAT having the sequence RKKRRORRRGC (SEQ ID NO:20) (Barnett et al., Invest. Ophthalmol. Vis. Sci. 2006, 47:2589-95). Yet other examples include VP22 protein from Herpes Simplex virus (Lundberg and Johansson, Biochem. Biophys. Res. Comm. 2002, 291:367-371), and the Pep-1 peptide carrier (Morris et al., Nature Biotech. 2001, 19:1173-76). In some embodiments, the peptides comprise D-isomer amino acids or other modifications, e.g., to improve uptake or reduce cellular degradation.

Polypeptides that include peptide mitochondrial-penetrating moieties can be produced by standard techniques, such as chemical synthesis, or expressed from a nucleic acid that encodes the polypeptide.

Other fragments that may be useful as mitochondrial-penetrating moieties include, but are not limited to, mitochondrial-targeting sequences that are found in proteins that localize to mitochondria. Non-limiting examples of mitochondrial-targeting sequences include the N-terminal region of human cytochrome c oxidase subunit VIII, the N-terminal region of the P1 isoform of subunit c of human ATP synthase, or the N-terminal region of the aldehyde dehydrogenase targeting sequence as described in U.S. Pat. App. 2004/0072774, herein incorporated by reference. For example, fragments of mitofusins (human mitofusin 1 sequence is at GenBank Acc. No. NP_{—}284941.2; human mitofusin 2 sequence is at GenBank Acc. No. NP_{—}055689.1), e.g., amino acids 97-757 of human mitofusin 2 (see U.S. Pat. No. 6,953,680, herein incorporated by reference), are useful as mitochondrial-targeting moieties in this invention.

Peptidomimetic Mitochondrial-Penetrating Moieties

Peptidomimetic mitochondrial penetrating moieties can also be used in the compositions and methods disclosed herein. A general description of peptidomimetics, and methods for making them, can be found above.

For example, non-hydrolyzeable tetraguanidinium compounds as described in Fernandez-Carneado et al., J. Am. Chem. Soc. 2005, 127:869-74, incorporated herein, can be used in the present compositions and methods.

Mitochondrial Targeting Signal Peptides

Fragments of that direct proteins to the mitochondria can also be used. Examples include RRIVVLHGYGAVKEVLLNHK (SEQ ID NO:31), amino acids 74-95 of Rat Cytochrome P450 2E1 (CYP2E1) (Neve and Ingelman-Sundberg, J. Biol. Chem. 2001, 276:11317-22; the cleavable prepiece from the yeast cytochrome c oxidase IV precursor (MLSLRQDIRFFKPATRTLCSSR (SEQ ID NO:32), see Maarse et al., EMBO J. 1984, 3:2831-37 and Hurt et al., FEBS 1984, 178:306-310); mitochondrial-targeting signal from the PB2 protein of influenza viruses (Carr et al., Virology 2006, 344:492-508; import signal contained within heme lyases (Diekert et al., Proc. Natl. Acad. Sci. U.S.A. 1999, 96:11752-57; the leader peptide of the mitochondrial matrix enzyme ornithine transcarbamylase (OTC) (Horwich et al., EMBO J. 1985, 4:1129-35; Hay et al., Biochim. Biophys. Acta 1984, 779:65-87; Fujiwara et al., Genome Inform. Ser. Workshop, Genome Inform. 1997, 8:53-60.

Nucleic Acid Mitochondrial-Penetrating Moieties

Nucleic acids that act as mitochondrial penetrating moieties (such as those described in U.S. Pat. No. 5,569,754, herein incorporated by reference, e.g., CCGCCAAGAAGCG (SEQ ID NO:21); GCGTGCACACGCGCGTAGACTTCC CCCGCAAGTCACTCGTTAGCCCGCCAAGAAGCGACCCCTCCGGGGCGAGCTG AGCGGCGTGGCGCGGGGGCGTCAT (SEQ ID NO:22); ACGTGCATACGCACGTAGACATTCCCCGCTTCCCACTCCAAAGTCCGCCAAG AAGCGTATCCCGCTGAGCGGCGTGGCGCGGGGGCGTCATCCGTCAGCTC (SEQ ID NO:23); or ACTTCCCCCGCAAGTCACTCGTTAGCCCGCCAAGAAG CGACCCCTCCGGGGCGAGCTG (SEQ ID NO:24) can also be used in the compositions and methods described herein. Methods for linking nucleic acids to peptides are known in the art.

Lipophilic Cation Mitochondrial-Penetrating Moieties

Lipophilic cations that act as mitochondrial penetrating moieties are described in Smith et al., Proc. Natl. Acad. Sci. U.S.A. 2003, 100:5407-12, which is herein incorporated by reference in its entirety. Lipophilic cations that are useful to this invention include, for example, Rhodamine 123 and phosphonium salts, e.g., methyltriphenylphosphonium and tetraphenylphosphonium.

In some embodiments, the cationic mitochondrial-penetrating moiety includes:

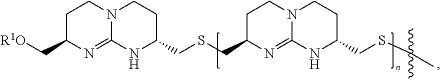

where R¹ is H, alkyl, alkenyl, alkynyl, haloalkyl, aryl, arylalkyl, or R^{a}R^{b}R^{c}Si; R^{a}, R^{b}, and R^{c} are independently selected from alkyl or aryl; and n can be 0, 1, 2, 3, 4, 5, or 6.

In some embodiments, the cationic mitochondrial-penetrating moiety includes

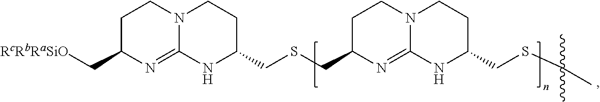

where, R^{a}, R^{b}, and R^{c} are independently selected from alkyl or aryl; and n can be 1, 2, or 3. In some embodiments, the cationic mitochondrial-penetrating moiety includes (aryl)₃P—. In some embodiments, the cationic mitochondrial-penetrating moiety includes Rhodamine 123:

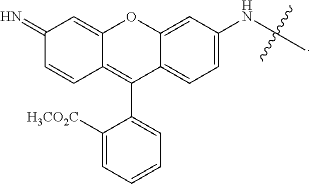

### IV. Linking Moiety

In some embodiments, the mitochondrial penetrating moiety is linked to a molecular chaperone inhibitor as described herein via a linker. As used herein, to “link” means to associate a mitochondrial-penetrating moiety and a chaperone inhibitor via a covalent or non-covalent bond or association.

A number of linkers can be used to link the chaperone inhibitor, to the mitochondrial-penetrating moiety. For example, a peptide linker can be used, e.g., a peptide linker including one, two, three, four, five, six, seven, eight, or more amino acids. In some embodiments, the peptide linker is flexible, i.e., contains amino acids that adopt flexible conformations, e.g., comprising glycine, alanine, and/or glutamine residues.

In embodiments where the mitochondrial-penetrating moiety and the chaperone inhibitor are both peptides, it will generally be desirable to produce the mitochondrial-targeted chaperone inhibitor as a fusion protein, with or without an intervening linker, e.g., using a nucleic acid that encodes the entire fusion protein.

In some embodiments, the linker moiety is divalent and can be selected from the group consisting of alkylene, alkenylene, alkynylene, cycloalkylene, arylene, heteroarylene, and peptide linker, wherein any two adjacent carbon-carbon bonds of said alkylene, alkenylene, or alkynylene, can be optionally replaced with one or more of O, NH, S, PR^{e}, C(O)NR^{f}, arylene, heterocycloalkylene, or heteroarylene; wherein R^{e} and R^{f} are independently selected from alkyl or aryl.

In some embodiments, the linker moiety is:

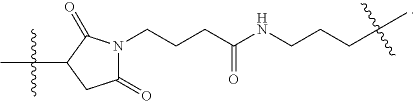

In some embodiments, the linker moiety is alkylene

In some embodiments, the linker moiety is alkylene with six carbon atoms.

One type of mitochondrial-targeted chaperone inhibitor is produced by crosslinking a chaperone inhibitor to a mitochondrial-penetrating moiety. Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

General methodology useful for making the compositions described herein are known in the art. In some embodiments, the methods can include contacting a mitochondrial-penetrating moiety, e.g., ANT as described herein, with a linker, e.g., a disulfide linker such as SSP, to form a reaction mixture, contacting the reaction mixture with a chaperone inhibitor, e.g., a geldanamycin analog, and obtaining a composition that includes a mitochondrial-penetrating moiety conjugated to the chaperone inhibitor. In some embodiments, the methods include contacting the mitochondrial-penetrating moiety with an amount of linker such that the ratio of linker to mitochondrial-penetrating moiety in the reaction mixture is about 1:1. Accordingly, the invention features methods of preparing a mitochondrial-penetrating moiety, e.g., ANT as described herein, conjugated to a chaperone inhibitor, e.g., a geldanamycin analog such as 17-AAG.

The mitochondrial-penetrating moiety and chaperone inhibitor can be joined, using recombinant methods known in the art, by a synthetic linker that enables them to be made as a single protein chain; see e.g., Bird et al., Science 1988, 242:423-426; and Huston et al., Proc. Natl. Acad. Sci. U.S.A. 1988, 85:5879-83.

For example, chaperone inhibitor of the invention can be functionally linked (by chemical coupling, genetic fusion, noncovalent association or otherwise) to one or more mitochondrial-penetrating moieties.

The compositions described herein include compounds of the formula:

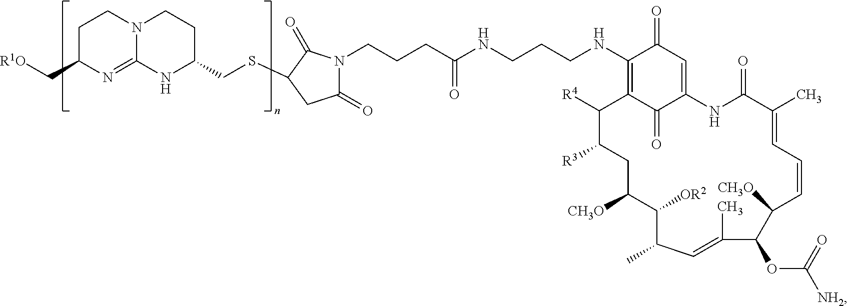

wherein, R¹ is H, alkyl, alkenyl, alkynyl, haloalkyl, aryl, arylalkyl, or R^{a}R^{b}R^{c}Si; R² is H, alkyl, aryl, or arylalkyl; R³ is H, alkyl; R⁴ is H, alkyl, alkenyl, aryl, arylalkyl, OR^{d}, wherein R^{d} is H, alkyl, or arylalkyl; R^{a}, R^{b}, and R^{c} are independently selected from alkyl or aryl; and n is an integer between 1 and 10, inclusive or a pharmaceutically acceptable salt thereof.

In some embodiments, the salt is a hexafluorophosphate salt.

In some embodiments, R¹ is R^{a}R^{b}R^{c}Si, R^{a}, R^{b}, and R^{c} are independently selected from alkyl or aryl; R² is H; R³ is H, alkyl; R⁴ is H; and n is 1, 2, 3, or 4.

In some embodiments, the compounds can be selected from:

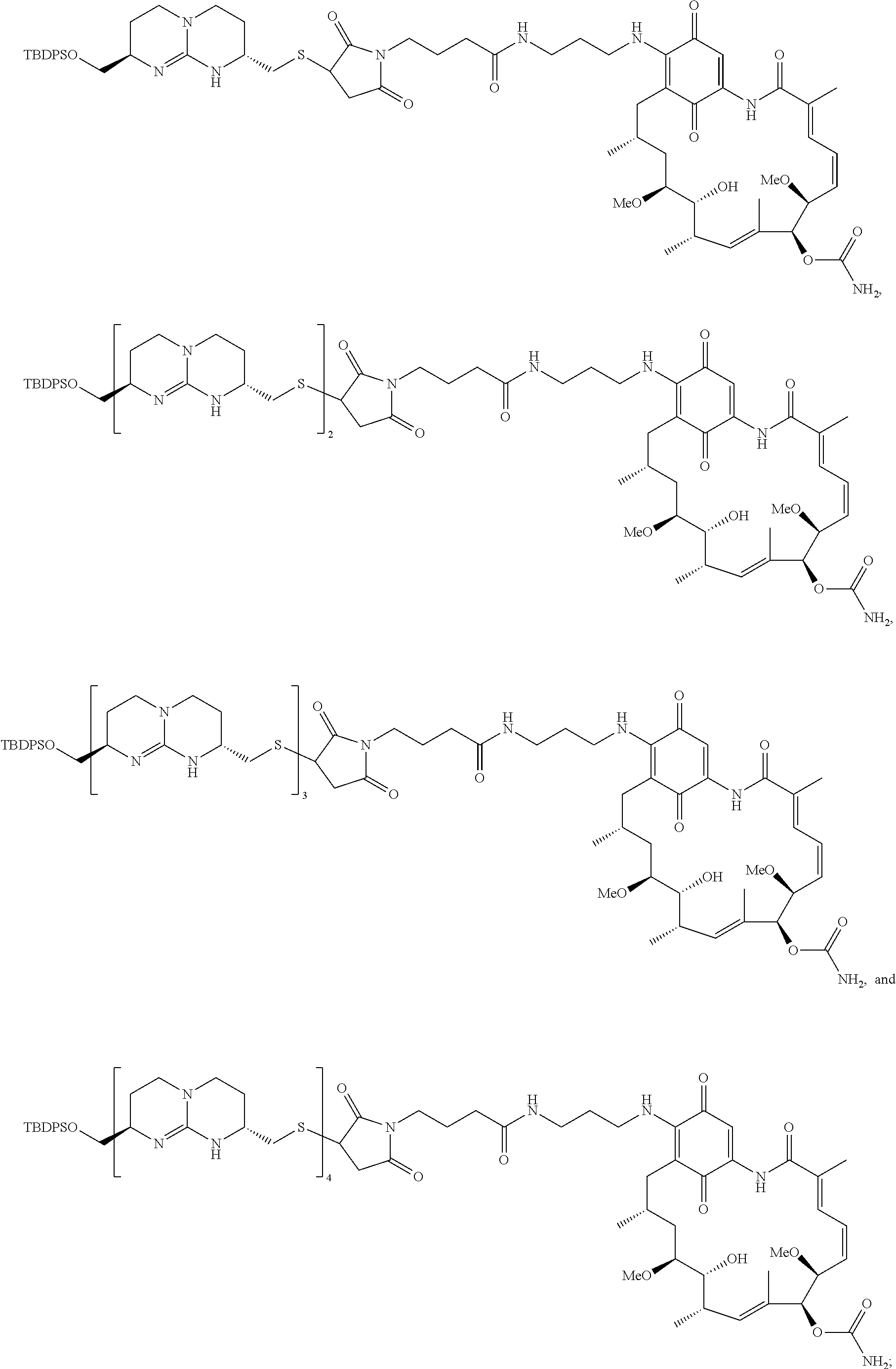

or a pharmaceutically acceptable salt thereof.

In some embodiments, the compounds can be of the formula:

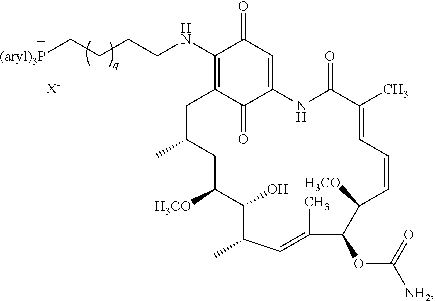

wherein, q is 1, 2, 3, 4, 5, or 6; and X is pharmaceutically acceptable counter-ion.

In some embodiments, q is 3.

In some embodiments, aryl is phenyl.

In some embodiments, aryl is phenyl and q is 3.

In some embodiments, X can be hexafluorophosphate.

In some embodiments, the compound can be:

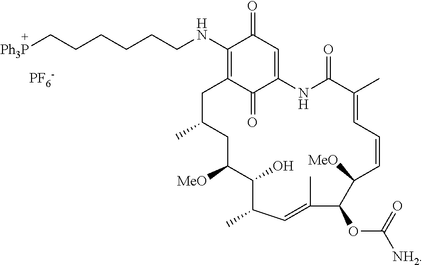

### Methods of Synthesis

The compounds described herein can be prepared by the conjugation of geldanamycin or the 17-GMB-APA-GA analogue. The use of the either of these compounds allows for conjugation with nucleophilic moieties such as thiols, amines, or alcohols. The elaboration of the cationic mitochondrial-penetrating moiety can be performed to include between one and 10 of the guanidinio moieties containing the monomeric structure:

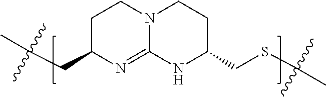

which can be abbreviated as

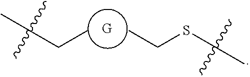

A general iterative method of synthesis of such compositions containing oligomeric guanidinio structures is shown below. The mesylate G1 can be treated with potassium acetate (KSAc) to provide the thio ester which upon base treatment followed by exposure to the maleimide derivative (L-GA) of a molecular chaperone inhibitor, such as geldanamycin (GA), can provide the desired first generation compositions G1-GA. The G1-thioester compound can in turn be treated in sequence such as with methanesulfonic acid; cesium carbonate in the presence of tributylphosphine; followed by reaction with G1 and finally treating with methanesulfonic anhydride provides G2 which is the higher homologue of G1. Such iterative process as can be seen clearly provides access to oligomeric guanidinio units. Few compounds are elaborated below in the Examples to demonstrate the process. Although the scheme shown here is described for the linkers with maleimide group for facilitating conjugation, this method can be extended to linkers with other functionalities for conjugation such as the N-hydroxy succinimide esters (such as 17-NHS-ALA-GA). One of skill in the art will also recognize that this iterative scheme can be extended to other non-GA based Hsp90 inhibitors such as the purine based antagonists or the resorcinol antagonists.

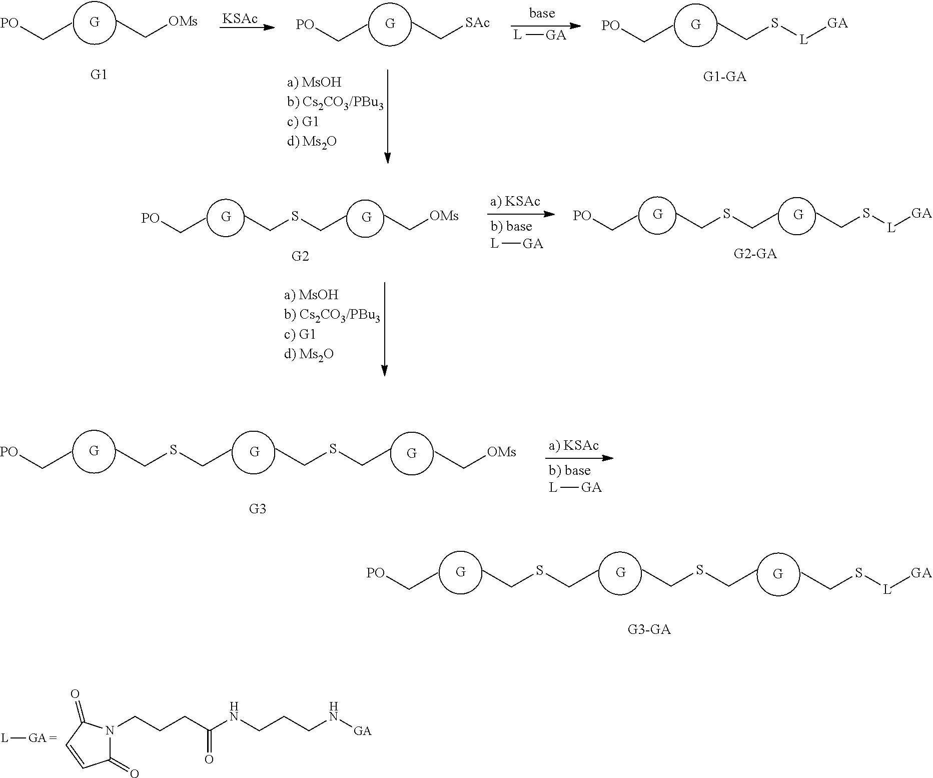

### V. Death Receptors

The death receptor family of signaling proteins are members of the TNF receptor superfamily that can induce apoptosis when bound by their respective ligands. The death receptors are characterized by a cytoplasmic region of ˜80 amino acid residues known as the death domain. When the receptors are triggered by their respective ligands, intracellular effectors are recruited to the death domain, activating a signaling cascade. There are eight known members of the death receptor family: TNF-related apoptosis-inducing ligand receptor 1 (TRAILR1), TRAILR2, TNF receptor 1 (TNFR1), CD95, lymphotoxin beta receptor (LTBR), death receptor 3 (DR3), DR6, ectodysplasin A receptor (EDAR), and nerve growth factor receptor (NGFR). A review of death receptor signaling can be found in Lavrik et al., J. Cell Sci. 2005, 118:265-267.

The following is a brief description of some of the death receptors that can be targeted using the present methods. In some embodiments, a death receptor polypeptide useful in the present methods is at least about 90%, 95%, 99%, or 100% identical to an amino acid sequence described herein (e.g., to a human sequence). In some embodiments, a nucleic acid encoding a death receptor useful in the present methods is at least about 90%, 95%, 99%, or 100% identical to a nucleic acid sequence described herein (e.g., to a human sequence).

TRAILR1 (TNF-Related Apoptosis-Inducing Ligand Receptor 1)

TRAILR1 is one of two known death receptors that bind to TRAIL/APO2L. Activation of TRAILR1 by TRAIL transduces a cell death signal and induces apoptosis. Overexpression of TRAILR1 induces apoptosis in a variety of transformed cell lines (Pan et al., Science 1997, 276:111-113).

GenBank Acc. Nos. for human TRAILR1 include NM_{—}003844.3 (nucleic acid) and NP_{—}003835.3 (protein). TRAILR1 is also known as DR4, APO-2, MGC9365, CD261, and tumor necrosis factor receptor superfamily, member 10A (TNFRSF10A). GenBank Acc. Nos. for human TRAIL/APO2L include NM_{—}003810.2 (nucleic acid) and NP_{—}003801.1 (protein). TRAIL/APO2L is also known as TL2, APO2L, CD253, TRAIL, Apo-2L, and tumor necrosis factor (ligand) superfamily, member 10 (TNFSF10). An exemplary human TRAIL/APO2L polypeptide has the sequence:

(SEQ ID NO: 17)
MAMMEVQGGPSLGQTCVLIVIFTVLLQSLCVAVTYVYFTNELKQMQDK
YSKSGIACFLKEDDSYWDPNDEESMNSPCWQVKWQLRQLVRKMILRTS
EETISTVQEKQQNISPLVRERGPQRVAAHITGTRGRSNTLSSPNSKNE
KALGRKINSWESSRSGHSFLSNLHLRNGELVIHEKGFYYIYSQTYFRF
QEEIKENTKNDKQMVQYIYKYTSYPDPILLMKSARNSCWSKDAEYGLY
SIYQGGIFELKENDRIFVSVTNEHLIDMDHEASFFGAFLVG

TRAILR2 (TNF-Related Apoptosis-Inducing Ligand Receptor 2)

TRAILR2 is one of two known death receptors that bind to TRAIL/APO2L. Activation of TRAILR2 by TRAIL transduces a cell death signal and induces apoptosis. Many glioblastoma cell lines mostly rely on DR5-mediated signaling since DR4 expression is silenced in many glioma cell lines by promoter hypermethylation.

GenBank Acc. Nos. for human TRAILR2 include NM_{—}003842.4 (nucleic acid) and NP_{—}003833.4 (protein); and NM_{—}147187.2 (nucleic acid) and NP_{—}671716.2 (protein). TRAILR2 is also known as DR5, CD262, KILLER, TRICK2, TRICKB, ZTNFR9, TRICK2A, TRICK2B, KILLER/DR5, and tumor necrosis factor receptor superfamily, member 10B (TNFRSF10B).

TNFR1 (TNF Receptor 1)

TNFR1 is a receptor for the tumor necrosis factor-alpha (TNF) ligand that mediates TNF-induced apoptosis. This protein is one of the major receptors for the tumor necrosis factor-alpha. TNFR1 can activate NF-κB, mediate apoptosis, and function as a regulator of inflammation.

GenBank Acc. Nos. for human TNFR1 include NM_{—}001065.2 (nucleic acid) and NP_{—}001056.1 (protein). TNFR1 is also known as DR1, FPF, p55, p60, TBP1, TNF-R, TNFAR, p55-R, CD120a, TNFR55, TNFR60, MGC19588, and tumor necrosis factor receptor superfamily, member 1A (TNFRSF1A). GenBank Acc. Nos. for human TNF include NM_{—}000594.2 (nucleic acid) and NP_{—}000585.2 (protein). TNF is also known as DIF, TNFA, TNF-alpha, and tumor necrosis factor (ligand) superfamily, member 2 (TNFSF2).

CD95

The interaction of this receptor with Fas ligand allows the formation of a death-inducing signaling complex that includes Fas-associated death domain protein (FADD), caspase 8, and caspase 10. The autoproteolytic processing of the caspases in the complex triggers a downstream caspase cascade, and leads to apoptosis.

GenBank Acc. Nos. for human CD95 include NM_{—}000043.3 (nucleic acid) and NP_{—}000034.1 (protein); and NM_{—}152871.1 (nucleic acid) and NP_{—}690610.1 (protein). CD95 is also known as FAS, APT1, FAS1, APO-1, FASTM, ALPS1A, FASDR1, and tumor necrosis factor receptor superfamily, member 6 (TNFRSF6). GenBank Acc. Nos. for human Fas ligand include NM_{—}000639.1 (nucleic acid) and NP_{—}000630.1 (protein). Fas ligand is also known as FASL, CD178, CD95L, APT1LG1, FASLG, and tumor necrosis factor (ligand) superfamily, member 6 (TNFSF6).

LTBR (Lymphotoxin Beta Receptor)

LTBR specifically binds the lymphotoxin membrane form (a complex of lymphotoxin-alpha and lymphotoxin-beta). Activation of the LBTR can trigger apoptosis.

GenBank Acc. Nos. for human LTBR include NM_{—}002342.1 (nucleic acid) and NP_{—}002333.1 (protein). LTBR is also known as CD18, TNFCR, D12S370, TNFR-RP, TNFR2-RP, LT-BETA-R, TNF-R-III, and tumor necrosis factor receptor superfamily, member 3 (TNFRSF3). GenBank Acc. Nos. for human lymphotoxin-beta include NM_{—}002341.1 (nucleic acid) and NP_{—}002332.1 (protein). Lymphotoxin-beta is also known as p33, TNFC, LTB, and tumor necrosis factor (ligand) superfamily, member 3 (TNFSF3).

DR3 (Death Receptor 3)

DR3 binds to the ligand TNFSF15/TL1A. This receptor is expressed preferentially in the tissues enriched in lymphocytes, and it may play a role in regulating lymphocyte homeostasis. DR3 has been shown to stimulate NF-kappa B activity and regulate cell apoptosis.

GenBank Acc. Nos. for human DR3 include NM_{—}148965.1 (nucleic acid) and NP_{—}683866.1 (protein); NM_{—}003790.2 (nucleic acid) and NP_{—}003781.1 (protein); NM_{—}148966.1 (nucleic acid) and NP_{—}683867.1 (protein); NM_{—}148967.1 (nucleic acid) and NP_{—}683868.1 (protein); NM_{—}148970.1 (nucleic acid) and NP_{—}683871.1 (protein). DR3 is also known as TR3, DDR3, LARD, APO-3, TRAMP, WSL1, WSL-LR, and tumor necrosis factor receptor superfamily, member 25 (TNFRSF25). GenBank Acc. Nos. for human TNFSF15/TL1A include NM_{—}005118.2 (nucleic acid) and NP_{—}005109.2 (protein). TNFSF15/TL1A is also known as TL1, TL1A, VEGI, VEGI192A, MGC129934, MGC129935, and tumor necrosis factor (ligand) superfamily, member 15 (TNFSF15).

DR6 (Death Receptor 6)

DR6 has been shown to activate NF-kappaB and MAPK8/JNK and induce cell apoptosis. GenBank Acc. Nos. for human DR6 include NM_{—}014452.3 (nucleic acid) and NP_{—}055267.1 (protein). DR6 is also known as BM-018, MGC31965, and tumor necrosis factor receptor superfamily, member 21 (TNFRSF21).

EDAR (Ectodysplasin A Receptor)

EDAR is a receptor for the soluble ligand ectodysplasin A, and can activate the nuclear factor-kappaB, JNK, and caspase-independent cell death pathways. GenBank Acc. Nos. for human EDAR include NM_{—}022336.3 (nucleic acid) and NP_{—}071731.1 (protein); and NM_{—}021783.2 (nucleic acid) and NP_{—}068555.1 (protein). EDAR is also known as DL, ED3, EDS, ED1R, EDA3, HRM1, EDA1R, EDA2R, EDA-A1R, FJ94390, and tumor necrosis factor receptor superfamily, member 27 (TNFRSF27). GenBank Acc. Nos. for human ectodysplasin A include NM_{—}001005609.1 (nucleic acid) and NP_{—}001005609.1 (protein); NM_{—}001005610.1 (nucleic acid) and NP_{—}001005610.1 (protein); NM_{—}001005611.1 (nucleic acid) and NP_{—}001005611.1 (protein); NM_{—}001005612.1 (nucleic acid) and NP_{—}001005612.1 (protein); NM_{—}001005615.1 (nucleic acid) and NP_{—}001005615.1 (protein); and NM_{—}001399.4 (nucleic acid) and NP_{—}001390.1 (protein).

NGFR (Nerve Growth Factor Receptor)

NGFR binds to nerve growth factor (NGF) and other neurotrophins, including brain-derived neurotrophic factor (BDNF), neurotrophin-3 (NT-3), and neurotrophin-4 (NT-4). GenBank Acc. Nos. for human NGFR include NM_{—}002507.2 (nucleic acid) and NP_{—}002498.1 (protein). NGFR is also known as CD271, p75NTR, p75(NTR), Gp80-LNGFR, and tumor necrosis factor receptor superfamily, member 16 (TNFRSF16). GenBank Acc. Nos. for human NGF include NM_{—}002506.2 (nucleic acid) and NP_{—}002497.2 (protein). NGF is also known as NGFB; HSAN5; Beta-NGF; MGC161426; and MGC161428.

### VI. Death Receptor Agonists

As used herein, a death receptor agonist is an agent that can activate signaling of a death receptor, e.g., to initiate apoptosis. When the receptors are triggered by an agonist, intracellular effectors can be recruited to the death domain, activating a signaling cascade. In some embodiments, a death receptor agonist is an agonist of a specific death receptor, e.g., an agonist of TRAILR1, TRAILR2, TNFR1, CD95, LTBR, DR3, DR6, EDAR, or NGFR.

Polypeptides

Death receptor agonists can be polypeptides, e.g., the natural ligands of death receptors and agonistic fragments (e.g., soluble fragments) and variants thereof. In some embodiments, a natural ligand of a death receptor is a human polypeptide. A ligand of a death receptor can be obtained from an animal species, e.g., a primate, monkey, rodent, canid, felid, bovine, ovine, or equine species. In some embodiments, a death receptor agonist useful in the present methods is at least about 90%, 95%, 99%, or 100% identical to a death receptor ligand amino acid sequence described herein (e.g., to a human sequence). In some embodiments, a nucleic acid encoding a death receptor useful in the present methods is at least about 90%, 95%, 99%, or 100% identical to a death receptor ligand nucleic acid sequence described herein (e.g., to a human sequence).

Additional peptide and peptide-containing agonists of TRAIL receptors are disclosed in U.S. Pat. No. 5,763,223 and US 2009/0131317, each of which is incorporated by reference in its entirety.

Fas receptor agonist AP0010 (TopoTarget, Copenhagen, Denmark) is a recombinant, soluble, hexameric fusion protein consisting of three human Fas ligand (FasL) extracellular domains fused to the dimer-forming collagen domain of human adiponectin with potential pro-apoptotic and antineoplastic activities. APO010 assembles into a soluble hexameric structure mimicking the ligand clustering of endogenous active FasL. Fas receptor agonist APO010 activates the Fas receptor, resulting in caspase-dependent apoptosis in susceptible tumor cell populations (Verbrugge et al., 2009, Clin. Cancer Res. 15:2031-38).

Additional Fas agonist peptides are disclosed in U.S. Pat. No. 6,001,962 and U.S. Pat. No. 6,846,637, each of which is incorporated by reference in its entirety.

Additional DR3 agonist peptides are disclosed in US 2008/0213837, which is incorporated by reference in its entirety.

Additional DR6 agonist peptides are disclosed in U.S. Pat. No. 6,423,494, which is incorporated by reference in its entirety.

In some embodiments, a soluble death receptor agonist or a soluble fragment of a death receptor agonist may be used in the methods and compositions disclosed herein. The soluble fragment can include all or a portion of an extracellular domain of a membrane-associated death receptor agonist. For example, an exemplary soluble TRAIL ligand can include amino acid residues 114-281 of SEQ ID NO:17. A soluble death receptor agonist or fragment thereof can be expressed, e.g., in a mammalian or bacterial cell, with its natural signal sequence or a heterologous signal sequence.

Variants of polypeptide death receptor agonists can also be used in the compositions and methods described herein. These include sequence variants. Where a conservative amino acid substitution is made, the substitution can be of one amino acid residue for another in any of the following groups: arginine, histidine, and lysine; aspartic acid and glutamic acid; alanine, leucine, isoleucine and valine; and phenylalanine, tryptophan and tyrosine. The amino acid residues listed here are naturally occurring. Non-naturally occurring amino acid residues of like kind may also be substituted. For example, a negatively charged non-naturally occurring amino acid residue may be substituted for a negatively charged naturally occurring amino acid residue; a hydrophobic aromatic non-naturally occurring amino acid residue may be substituted for a hydrophobic aromatic naturally occurring amino acid residue; and so forth.

The degree of identity can vary and can be determined by methods well established in the art. “Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. A biologically active variant of a polypeptide described herein can have at least or about 80%, 85%, 90%, 95%, 96%, 97%, 98%, or 99% identity or homology to a corresponding naturally occurring polypeptide. The nucleic acids encoding the biologically active variant polypeptides can be similarly described as having at least or about 80%, 85%, 90%, 95%, 96%, 97%, 98%, or 99% identity to a corresponding naturally occurring nucleic acid sequence. Those of ordinary skill in the art will readily recognize degenerate variants of nucleic acid sequences, and such variants can be used for the purposes described herein.

When using a polypeptide death receptor agonist in a human subject, it will generally be desirable to use a human or humanized sequence. Thus, the methods described herein can include using standard molecular biology techniques to humanize a non-human sequence. Alternatively, human sequences can be used to make the construct.

Modified versions of the polypeptides described herein can also be used in the compositions and methods described herein. The polypeptides and biologically active variants thereof can be modified in numerous ways. For example, agents, including additional amino acid residues, other substituents, and protecting groups can be added to either the amino terminus, the carboxy terminus, or both. The modification can be made for the purpose of altering the peptides' form or altering the way the peptides bind to or interact with one another, with non-identical peptides, or with other polypeptides. For example, the peptides can be modified to include cysteine residues or other sulphur-containing residues or agents that can participate in disulphide bond formation. For example, one can add at least two cysteine residues, one or both of which are, optionally, at the C-terminal or N-terminal of the peptide.

In some instances, the polypeptides can further include a substituent at the amino-terminus or carboxy-terminus. The substituent can be an acyl group or a substituted or unsubstituted amine group (e.g., the substituent at the N-terminus can be an acyl group and the C-terminus can be amidated with a substituted or unsubstituted amine group (e.g., an amino group having one, two, or three substituents, which may be the same or different)). The amine group can include a lower alkyl (e.g., an alkyl having 1-4 carbons), alkenyl, alkynyl, or haloalkyl group. The acyl group can be a lower acyl group (e.g., an acyl group having up to four carbon atoms), especially an acetyl group.

As noted, the polypeptides can vary in length and can be or can include contiguous amino acid residues that naturally occur in death receptor agonists, or that vary to a certain degree from naturally occurring death receptor agonist sequences (but retain sufficient activity to be useful). Where the polypeptides include, at their N-terminus or C-terminus (or both), amino acid residues that are not naturally found in death receptor agonists, the additional sequence(s) can be about 200 amino acid residues long, and these residues can be divided evenly or unevenly between the N- and C-termini. For example, both the N- and C-termini can include about 10, 20, 30, 40, 50, 60, 70, 80, 90, or 100 amino acid residues. Alternatively, one terminus can include about 10, 20, 30, 40, 50, 60, 70, 80, 90, 100, 110, 120, 130, 140, 150, 160, 170, 180, 190, or 200 residues, and one terminus can include none.

The polypeptides, including the modified polypeptides described above, can be protease resistant and can include one or more types of protecting groups such as an acyl group, an amide group, a benzyl or benzoyl group, or a polyethylene glycol. More specifically, a peptide, including the modified polypeptides described above, can be N-terminally acetylated and/or C-terminally amidated.

Where non-naturally occurring or modified amino acid residues are included they can be selected from the following or many others available in the art: 4-hydroxyproline, gamma-carboxyglutamic acid, o-phosphoserine, o-phosphotyrosine, or delta-hydroxylysine. Other examples include naphthylalanine, which can be substituted for trytophan to facilitate synthesis, L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, and isoquinolyl. Peptides having non-naturally occurring amino acid residues may be referred to as synthetic peptides and constitute one type of variant as described herein. Other variants include peptides in which a naturally occurring side chain of an amino acid residue (in either the L- or D-form) is replaced with a non-naturally occurring side chain.

In another embodiment, the polypeptides can be PEGylated by methods known in the art.

For guidance on peptide modification by reduction/alkylation and/or acylation, one can consult Tarr, Methods of Protein Microcharacterization, J. E. Silver ed., Humana Press, Clifton N.J. (1986) 155-194; for guidance on chemical coupling to an appropriate carrier, one can consult Mishell and Shiigi, eds, Selected Methods in Cellular Immunology, WH Freeman, San Francisco, Calif. (1980) and U.S. Pat. No. 4,939,239; and for guidance on mild formalin treatment, one can consult Marsh, Int. Arch. Allergy Appl. Immunol. (1971) 41:199-215.

Peptidomimetics of the inhibitory peptides can also be used. Peptide inhibitors disclosed herein and known in the art can be modified according to methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem. 278:45746, (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as ∀-amino phosphonic acids and ∀-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. The sequences can be modified, e.g., by biotinylation of the amino terminus and amidation of the carboxy terminus.

Any of the peptides described herein, including the variant forms described herein, can further include a heterologous polypeptide (e.g., a polypeptide having a sequence that does not appear in a death receptor agonist). The heterologous polypeptide can be a polypeptide that increases the circulating half-life of the peptide to which it is attached (e.g., fused, as in a fusion protein). The heterologous polypeptide can be an albumin (e.g., a human serum albumin or a portion thereof) or a portion of an immunoglobulin (e.g., the Fc region of an IgG).

Compounds mimicking the necessary conformation of the peptides described herein are contemplated as within the scope of this invention. A variety of designs for such mimetics are possible. U.S. Pat. No. 5,192,746; U.S. Pat. No. 5,169,862; U.S. Pat. No. 5,539,085; U.S. Pat. No. 5,576,423; U.S. Pat. No. 5,051,448; and U.S. Pat. No. 5,559,103, all hereby incorporated by reference, describe multiple methods for creating such compounds.

Non-peptidic compounds that mimic peptide sequences are known in the art (Meli et al., 2006, J. Med. Chem. 49:7721-30, describing methods of identifying nonpeptide small molecule mimics of shepherdin). Synthesis of non-peptide compounds that mimic peptide sequences is also known in the art (see, e.g., Eldred et al., 1994, J. Med. Chem. 37:3882; Ku et al., 1995, J. Med. Chem. 38:9; Meli et al., 2006, J. Med. Chem. 49:7721-30). Such nonpeptide compounds that mimic CBP peptides that bind chaperones are specifically contemplated by the present invention.

The present invention also contemplates synthetic mimicking compounds. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the (α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In one embodiment, the mimetics of the present invention are peptides having sequence homology to the herein-described chaperone inhibitor peptides. These mimetics include, but are not limited to, peptides in which L-amino acids are replaced by their D-isomers. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant (Pearson and Lipman, 1988, Proc. Natl. Acad. Sci. U.S.A. 85:2444-48; Lipman and Pearson, 1985, Science 227:1435-41). More generally, the CBP peptides described herein and the mimetics described above can be synthesized using any known methods, including tea-bag methodology or solid phase peptide synthesis procedures described by Merrifield et al., 1982, Biochemistry 21:5020-31; Houghten, 1985, Proc. Natl. Acad. Sci. U.S.A. 82:5131-35; Atherton, 1997, Meth. Enzymol. 289:44-66; or Guy and Fields, 1997, Meth. Enzymol. 289:67-83, or using a commercially available automated synthesizer.

Antibodies

Another class of death receptor agonists that can be used in the methods and compositions described herein is agonistic antibodies to death receptors. Such agonistic antibodies can be anti-TRAILR1 antibody, anti-TRAILR2 antibody, anti-TNFR1 antibody, anti-CD95 antibody, anti-LTBR antibody, anti-DR3 antibody, anti-DR6 antibody, anti-EDAR antibody, or anti-NGFR antibody. In some embodiments, the agonistic antibodies are selected from the group consisting of anti-TRAIL-R1 antibody, anti-TRAIL-R2 antibody, anti TNF-R1 antibody, and fragments and derivatives of any of said antibodies.

The term “antibody,” as used herein, refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody. Antibodies that immunospecifically bind to death receptors can be identified, for example, by immunoassays or other techniques known to those of skill in the art.

Antibodies useful in the methods and compositions disclosed herein include, but are not limited to, monoclonal, multispecific, human or chimeric antibodies, single chain antibodies, Fab fragments, F(ab′) fragments, anti-idiotypic (anti-Id) antibodies (including, e.g., anti-Id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. In some embodiments, the immunoglobulin is an IgG1 isotype. In other embodiments, the immunoglobulin is an IgG4 isotype. Immunoglobulins may have both a heavy and light chain.

Antibodies of the invention may also include multimeric forms of antibodies. For example, antibodies of the invention may take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers, and other higher-order antibody multimers. Alternatively, antibody homodimers may be formed through chemical linkage techniques known in the art. For example, heterobifunctional crosslinking agents including, but not limited to, SMCC [succinimidyl 4-(maleimidomethyl)cyclohexane-1-carboxylate] and SATA [N-succinimidyl S-acethylthio-acetate] (available, for example, from Pierce Biotechnology, Inc. (Rockford, Ill.)) can be used to form antibody multimers. An exemplary protocol for the formation of antibody homodimers is given in Ghetie et al., Proc. Natl. Acad. Sci. USA (1997) 94:7509-14, which is hereby incorporated by reference in its entirety. Antibody homodimers can be converted to Fab′2 homodimers through digestion with pepsin. Another way to form antibody homodimers is through the use of the autophilic T15 peptide described in Zhao and Kohler, 2002, J. Immunol. 25:396-404, which is hereby incorporated by reference in its entirety.

Alternatively, antibodies can be made to multimerize through recombinant DNA techniques. IgM and IgA naturally form antibody multimers through the interaction with the mature J chain polypeptide. Non-IgA or non-IgM molecules, such as IgG molecules, can be engineered to contain the J chain interaction domain of IgA or IgM, thereby conferring the ability to form higher order multimers on the non-IgA or non-IgM molecules (see, for example, Chintalacharuvu et al., 2001, Clin. Immunol. 101:21-31 and Frigerio et al., 2000, Plant Physiol. 123:1483-94). IgA dimers are naturally secreted into the lumen of mucosa-lined organs. This secretion is mediated through interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (Johansen et al., 2001, J. Immunol., 167:5185-92 which is hereby incorporated by reference in its entirety). ScFv dimers can also be formed through recombinant techniques known in the art; an example of the construction of scFv dimers is given in Goel et al., 2000, Cancer Res. 60:6964-71 which is hereby incorporated by reference in its entirety. Antibody multimers may be purified using any suitable method known in the art, including, but not limited to, size exclusion chromatography.

An exemplary anti-TRAILR1 agonistic antibody that can be used in the methods and compositions described herein is mapatumumab (Human Genome Sciences, Inc.). Exemplary anti-TRAILR2 agonistic antibodies that can be used in the methods and compositions described herein include lexatumumab (Human Genome Sciences, Inc.), conatumumab (AMG-655; Amgen, Inc.), and apomab (Genentech). Other exemplary anti-TRAIL receptor agonistic antibodies are described in U.S. Pat. No. 7,361,341; U.S. Pat. No. 7,348,003; U.S. Pat. No. 7,064,189; U.S. Pat. No. 7,476,383; U.S. Pat. No. 7,476,384; U.S. Pat. No. 7,279,160; U.S. Pat. No. 7,521,048; U.S. Pat. No. 7,244,429; and US 2003/0228309, all of which are incorporated by reference in their entirety.

Exemplary anti-TNFR1 agonistic antibodies are described in Totpal et al., 1994, J. Immunol. 53:2248-57, which is incorporated by reference in its entirety.

Exemplary anti-CD95 agonistic antibodies are described in U.S. Pat. No. 589,143 and U.S. Pat. No. 6,846,637, each of which is incorporated by reference in its entirety.

Exemplary anti-LTBR agonistic antibodies are described in Lukashev et al., 2006, Cancer Res. 66:9617-24; U.S. Pat. No. 6,312,691; U.S. Pat. No. 7,001,598; U.S. Pat. No. 7,429,645; U.S. Pat. No. 7,459,537; US 2006/0222644; and US 2009/0123421, all of which are incorporated by reference in their entirety.

Small Molecules

Small molecule death receptor agonists useful in the methods and compositions described herein are known in the art. For example, small molecule death receptor agonists that are useful in the compositions and methods described herein include, but are not limited to, molecules that bind to a ligand binding region of a death receptor. Small molecule death receptor agonists known in the art are described, for example, in Hymowitz and Ashkenazi, 2005, Nat. Chem. Biol. 1:353-354; Saragovi and Zaccaro, 2002, Curr. Pharm. Des. 8:2201-16; and US 2008/0214547.

Additional small molecule agonists of death receptors can be identified using computer aided drug design (CADD) by screen a virtual library, e.g., of commercially available small molecular weight compounds, to identify ligands that bind to a death receptor. In silico screening can targeted a region of the death receptor that interacts directly with its ligand based on the crystallographic structure. From the in silico screen, compounds can be obtained for biological assay. The compounds can be further modified and tested in cell culture and in vivo animal experiments for antitumor activity. In addition to CADD, chemical libraries of small molecular weight organic compounds can be screened by high throughput assay to identify death receptor agonists.

### VII. Autophagy Inhibitors

Autophagy inhibitors used in the methods described herein can be any molecule (e.g., protein, small organic or inorganic molecule, or nucleic acid, such as an antisense oligonucleotide, ribozyme, or siRNA) that can decrease or inhibit autophagy in a cell (e.g., a cancer or tumor cell). Non-limiting examples of autophagy inhibitors are described herein. Additional autophagy inhibitors are known in the art.

Non-limiting examples of autophagy inhibitors include the small molecules of: 3-methyladenine, bafilomycin A1, LY294002, wortmanin, hydroxychloroquine, chloroquine, 5-amino-4-imidazole carboxamide riboside, okadaic acid, a microcystin, microeystin, nodularin, analogues of cAMP, agents that elevate cAMP levels, adenosine, N6-mercaptopurine riboside, wortmannin, and vinblastine. Additional examples of autophagy inhibitors are antisense oligonucleotides, ribozymes, and siRNAs that decrease the expression of MAP1LC3B (NCBI Accession No. NM_{—}022818.4), HSP90AA1 (NCBI Accession No. NM_{—}001017963.2 or NM_{—}005348.3), HSPA8 (NCBI Accession No. NM_{—}006597.3 or NM_{—}153201.1), AMBRA1 (NCBI Accession No. BC045609.1 or NM_{—}017749.2), ATG12 (NCBI Accession No. NM_{—}004707.3, NR_{—}033362.1, or NR_{—}033363.1), ATG16L1 (NCBI Accession No. NM_{—}030803.6, NM_{—}017974.3, NM_{—}198890.2, NM_{—}001190266.1, or NM_{—}001190267.1), ATG4A (NCBI Accession No. NM_{—}052936.3 or NM_{—}178270.2), ATG4B (NCBI Accession No. NM_{—}013325.4 or NM_{—}178326.2), ATG4C(NCBI Accession No. NM_{—}032852.3 or NM_{—}178221.2), ATG4D (NCBI Accession No. NM_{—}032885.4), ATG5 (NCBI Accession No. NM_{—}004849.2, EU283339.1, or EU283338.1), ATG9A (NM_{—}001077198.1 or NM_{—}024085.3), ATG9B (NCBI Accession No. NM_{—}173681.5 or BC128587.1), BECN1 (NCBI Accession No. NM_{—}003766.3 or AF139131.1), GABARAP (NCBI Accession No. NM_{—}007278.1), GABARAPL1 (NCBI Accession No. NM_{—}031412.2, AF087847.1, or AY422178.1), GABARAPL2 (NCBI Accession No. NM_{—}007285.6 or AF087848.1), IRGM (NCBI Accession No. NM_{—}001145805.1 or EU742619.1), MAP1LC3A (NCBI Accession No. NM_{—}032514.2 or NM_{—}181509.1), RGS1 (NCBI Accession No. NM_{—}002922.3 or AF493925.1), ULK1 (NCBI Accession No. NM_{—}003565.2), ATG10 (NCBI Accession No. BC029268.1, NM_{—}001131028.1, or NM_{—}031482.4), ATG16L2 (NCBI Accession No. NM_{—}033388.1, BC036713.2, BC137490.1, or BC146660.1), ATG3 (NCBI Accession No. NM_{—}022488.3 or NG_{—}007378.2), ATG7 (NCBI Accession No. NM_{—}006395.2, NM_{—}001136031.2, or NM_{—}001144912.1), RAB24 (NCBI Accession No. NM_{—}001031677.2, NM_{—}130781.2, BC010006.2, AF087904.1, or BC015534.1), DRAM (NCBI Accession No. NM_{—}178454.4 or NM_{—}018370.2), TMEM166 (NCBI Accession No. NM_{—}032181.2 or NM_{—}001135032.1), AKT1 (NCBI Accession No. NM_{—}005163.2 or NM_{—}001014432.1), APP (NCBI Accession No. NM_{—}000484.3, NM_{—}201413.2, NM_{—}201414.2, NM_{—}001136016.3, NM_{—}001136129.2, NM_{—}001136130.2, NM_{—}001136131.2, NM_{—}001204301.1, NM_{—}001204302.1, or NM_{—}001204303.1), BAD (NCBI Accession No. NM_{—}004322.3 or NM_{—}032989.2), BAK1 (NCBI Accession No. NM_{—}001188.3), BAX (NCBI Accession No. NR_{—}027882.1, NM_{—}138764.4, NM_{—}138763.3, NM_{—}004324.3, or NM_{—}138761.3), BCL2 (NCBI Accession No. NM_{—}000657.2 or NM_{—}000633.2), BCL2L1 (NCBI Accession No. NM_{—}138578.1 or NM_{—}001191.2), BID (NCBI Accession No. NM_{—}197967.1, NM_{—}197966.1, or NM_{—}001196.2), BNIP3 (NCBI Accession No. U15174.1), CASP3 (NCBI Accession No. NM_{—}004346.3 or BC016926.2), CASP8 (NCBI Accession No. NM_{—}001080125.1, NM_{—}001080124.1, NM_{—}033358.3, NM_{—}033356.3, NM_{—}001228.4, or NM_{—}033355.3), CDKN1B (NCBI Accession No. NM_{—}004064.3), CDKN2A (NCBI Accession No. NM_{—}058195.3, NM_{—}001195132.1, NM_{—}058197.4, or NM_{—}000077.4), CLN3 (NCBI Accession No. NM_{—}000086.2 or NM_{—}001042432.1), CTSB (NCBI Accession No. NM_{—}001908.3, NM_{—}147780.2, NM_{—}147781.2, NM_{—}147782.2, or NM_{—}147783.2), CXCR4 (NCBI Accession No. NM_{—}001008540.1 or NM_{—}003467.2), DAPK1 (NCBI Accession No. NM_{—}004938.2), EIF2AK3 (NCBI Accession No. NM_{—}004836.5), FADD (NCBI Accession No. NM_{—}003824.3), FAS (NCBI Accession No. NM_{—}000043.4), HDAC1 (NCBI Accession No. AY893466.1 or AY893265.1), HTT (NCBI Accession No. NM_{—}002111.6), IFNA2 (NCBI Accession No. NM_{—}000605.3), IFNG (NCBI Accession No. NM_{—}000619.2), IGF1 (NCBI Accession No. NM_{—}001111283.1, NM_{—}001111284.1, NM_{—}001111285.1, or NM_{—}000618.3), INS(NCBI Accession No. NM_{—}000207.2, NM_{—}001185097.1, or NM_{—}001185098.1), MAPK8 (NCBI Accession No. NM_{—}139049.1, NM_{—}139047.1, NM_{—}139046.1, or NM_{—}002750.2), NFKB1 (NCBI Accession No. NM_{—}003998.3 or NM_{—}001165412.1), PIK3CG (NCBI Accession No. NM_{—}002649.2), PRKAA1 (NCBI Accession No. NM_{—}006251.5 or NM_{—}206907.3), PTEN (NCB I Accession No. NM_{—}000314.4), SNCA (NCBI Accession No. NM_{—}000345.3, NM_{—}007308.2, NM_{—}001146054.1, or NM_{—}001146055.1), SQSTM1 (NCBI Accession No. NM_{—}003900.4, NM_{—}001142298.1, or NM_{—}001142299.1), TGFB1 (NCBI Accession No. NM_{—}000660.4), TGM2 (NCBI Accession No. NM_{—}004613.2 or NM_{—}198951.1), TNF (NCBI Accession No. NM_{—}000594.2), TNFSF10 (NCBi Accession No. NM_{—}003810.3, NM_{—}001190942.1, NM_{—}001190943.1, or NR_{—}033994.1), TP53 (NCBI Accession No. NM_{—}000546.4, NM_{—}001126112.1, NM_{—}001126113.1, NM_{—}001126114.1, NM_{—}001126115.1, NM_{—}001126116.1, or NM_{—}001126117.1), TP73 (NCBI Accession No. NM_{—}001204186.1, NM_{—}001204189.1, NM_{—}005427.3, NM_{—}001204184.1, NM_{—}001204187.1, NM_{—}001204185.1, NM_{—}001204188.1, NM_{—}001126240.2, NM_{—}001126241.2, NM_{—}001204190.1, NM_{—}001204191.1, or NM_{—}001204192.1), CDKN2A (NCBI Accession No. NM_{—}058195.3, NM_{—}001195132.1, NM_{—}058197.4, or NM_{—}000077.4), IFNG (NCBI Accession No. NM_{—}000619.2), RB1 (NCBI Accession No. NM_{—}000321.2), IFNA2 (NCBI Accession No. NM_{—}000605.3), IFNA4 (NCBI Accession No. NM_{—}021068.2), IFNG (NCBI Accession No. NM_{—}000619.2), ARSA (NCBI Accession No. NM_{—}000487.5, NM_{—}001085425.2, NM_{—}001085426.2, or NM_{—}001085427.2), CTSS(NCBI Accession No. NM_{—}004079.4 or NM_{—}001199739.1), EIF4G1 (NCBI Accession No. NM_{—}182917.4, NM_{—}198242.2, NM_{—}001194946.1, NM_{—}001194947.1, NM_{—}198241.2, NM_{—}004953.4, or NM_{—}004953.4), ESR1 (NCBI Accession No. NM_{—}000125.3, NM_{—}001122740.1, NM_{—}001122741.1, or NM_{—}001122742.1), GAA (NCBI Accession No. NM_{—}000152.3, NM_{—}001079803.1, or NM_{—}001079804.1), HGS (NCBI Accession No. NM_{—}004712.4), MAPK14 (NCBI Accession No. NM_{—}139012.2, NM_{—}139014.2, NM_{—}001315.2, or NM_{—}139013.2), PIK3C3 (NCBI Accession No. NM_{—}002647.2), PIK3R4 (NCBI Accession No. NM_{—}014602.2), PRKAA2 (NCBI Accession No. NM_{—}006252.3), RPS6KB1 (NCBI Accession No. NM_{—}003161.2), TMEM74 (NCBI Accession No. NM_{—}153015.1), TMEM77 (NCBI Accession No. BC091509.1 or BC047025.1), ULK2 (NCBI Accession No. NM_{—}014683.3 or NM_{—}001142610.1), or UVRAG (NCBI Accession No. NM_{—}003369.3) in a cell (e.g., a cancer or tumor cell). The exemplary mRNA sequences described above for each gene are non-limiting. Additional mRNA sequences for several of the above genes are known in the art. Exemplary methods for the design of antisense oligonucleotides, ribozymes, and siRNA to decrease the expression of any of the genes described herein are described below. Additional methods for the design of antisense oligonucleotides, ribozymes, and siRNA are known in the art.

### Antisense Nucleic Acids

Agents to modulate the expression of any of the genes described herein include antisense nucleic acid molecules, i.e., nucleic acid molecules whose nucleotide sequence is complementary to all or part of an mRNA based on the sequence of a gene (e.g., based on a sequence of any of the genes described herein). An antisense nucleic acid molecule can be antisense to all or part of a non-coding region or the coding strand of a nucleotide sequence encoding the any of the genes described herein. Non-coding regions (“5′ and 3′ untranslated regions”) are the 5′ and 3′ sequences that flank the coding region in a gene and are not translated into amino acids.

Based upon the mRNA sequences disclosed herein, one of skill in the art can easily choose and synthesize any of a number of appropriate antisense molecules to target a gene described herein. For example, a “gene walk” comprising a series of oligonucleotides of 15-30 nucleotides spanning the length of any nucleic acid described herein can be prepared, followed by testing for inhibition of expression of the gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein can be prepared in vitro and administered to an animal, e.g., a mammal, e.g., a human patient. Alternatively, they can be generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol II or pol III promoter.

An antisense nucleic acid molecule of the invention can be an α-anomeric nucleic acid molecule. An α-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid molecule can also comprise a 2′-o-methylribonucleotide (Inoue et al., Nucleic Acids Res. 15:6131-6148, 1987) or a chimeric RNA-DNA analog (Inoue et al., FEBS Lett. 215:327-330, 1987).

### Ribozymes

Also provided are ribozymes that have specificity for sequences encoding the any of the genes described herein. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. A ribozyme having specificity for a nucleic acid molecule of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a glucose transport-related mRNA (Cech et al. U.S. Pat. No. 4,987,071; and Cech et al., U.S. Pat. No. 5,116,742). Alternatively, an mRNA encoding a polypeptide of the invention can be used to select a catalytic RNA having a specific ribonuclease activity from a pool of RNA molecules. See, e.g., Bartel and Szostak, Science 261:1411-1418, 1993.

Also provided herein are nucleic acid molecules that form triple helical structures. For example, expression of any of the genes described herein can be inhibited by targeting nucleotide sequences complementary to the regulatory region of the gene encoding the polypeptide (e.g., the promoter and/or enhancer) to form triple helical structures that prevent transcription of the gene in target cells. See generally Helene, Anticancer Drug Des. 6(6):569-84, 1991; Helene, Ann. N.Y. Acad. Sci. 660:27-36, 1992; and Maher, Bioassays 14(12):807-15, 1992.

In various embodiments, nucleic acid molecules (e.g., nucleic acid molecules used to modulate expression of any of the genes described herein) can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al., Bioorganic & Medicinal Chem. 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. U.S.A. 93: 14670-675, 1996.

PNAs can be used in therapeutic applications. For example, PNAs can be used as antisense or antigene agents for sequence-specific modulation of gene expression by, e.g., inducing transcription or translation arrest or inhibiting replication. PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res. 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res. 17:5973-88, 1989). PNA monomers are then coupled in a stepwise manner to produce a chimeric molecule with a 5′ PNA segment and a 3′ DNA segment (Finn et al., Nucleic Acids Res. 24:3357-63, 1996). Alternatively, chimeric molecules can be synthesized with a 5′ DNA segment and a 3′ PNA segment (Peterser et al., Bioorganic Med. Chem. Lett. 5:1119-11124, 1975).

In some embodiments, the oligonucleotide includes other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556, 1989; Lemaitre et al., Proc. Natl. Acad. Sci. U.S.A. 84:648-652, 1989; WO 88/09810) or the blood-brain barrier (see, e.g., WO 89/10134). In addition, oligonucleotides can be modified with hybridization-triggered cleavage agents (see, e.g., Krol et al., Bio/Techniques 6:958-976, 1988) or intercalating agents (see, e.g., Zon, Pharm. Res. 5:539-549, 1988). To this end, the oligonucleotide may be conjugated to another molecule, e.g., a peptide, hybridization triggered cross-linking agent, transport agent, hybridization-triggered cleavage agent, etc.

siRNA

Another means by which expression of any of the above described genes can be inhibited is by RNA interference (RNAi). RNAi is a process in which mRNA is degraded in host cells. To inhibit an mRNA, double-stranded RNA (dsRNA) corresponding to a portion of the gene to be silenced (e.g., a gene encoding any of the polypeptides described herein) is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev. 15:485-490, 2001, and Hammond et al., Nature Rev. Gen. 2:110-119, 2001).

RNA-mediated gene silencing can be induced in mammalian cells in many ways, e.g., by enforcing endogenous expression of RNA hairpins (see Paddison et al., Proc. Natl. Acad. Sci. U.S.A. 99:1443-1448, 2002) or, as noted above, by transfection of small (21-23 nt) dsRNA (reviewed in Caplen, Trends Biotech. 20:49-51, 2002). Methods for modulating gene expression with RNAi are described, e.g., in U.S. Pat. No. 6,506,559 and U.S. Pat. Pub. No. 20030056235, which are hereby incorporated by reference.

Standard molecular biology techniques can be used to generate siRNAs. Short interfering RNAs can be chemically synthesized, recombinantly produced, e.g., by expressing RNA from a template DNA, such as a plasmid, or obtained from commercial vendors such as Dharmacon. The RNA used to mediate RNAi can include synthetic or modified nucleotides, such as phosphorothioate nucleotides. Methods of transfecting cells with siRNA or with plasmids engineered to make siRNA are routine in the art.

The siRNA molecules used to modulate expression of any of the polypeptides described herein can vary in a number of ways. For example, they can include a 3′ hydroxyl group and strands of 21, 22, or 23 consecutive nucleotides. They can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand.

To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). Any siRNA can be used in the methods described herein, provided it has sufficient homology to the target of interest. There is no upper limit on the length of the siRNA that can be used (e.g., the siRNA can range from about 21 base pairs of the gene to the full length of the gene or more (e.g., 50-100, 100-250, 250-500, 500-1000, or over 1000 base pairs).

### VIII. NF-κB Signaling Pathway Inhibitors

NF-κB signaling pathway inhibitors used in the methods described herein can be any molecule that decreases one or more (e.g., two, three, or four) of the following in a cell (e.g., a tumor or cancer cell): the expression levels of NF-κB (protein or mRNA), activation of a cellular receptor tyrosine kinase that is upstream of NF-κB activation, activation of a cytosolic kinase that is upstream of NF-κB, phosphorylation and/or degradation of IκBα, nuclear translocation of NF-κB into the nucleus, NF-κB binding to KB promoter elements, and NF-κB transactivation of gene transcription. Non-limiting examples of NF-κB signaling pathway inhibitors are described herein. Additional NF-κB signaling pathway inhibitors are known in the art.

A variety of antioxidant molecules can be used as NF-κB signaling pathway inhibitors. Non-limiting examples of such antioxidant molecules include: α-lipoic acid, α-tocopherol, allicin, 2-amino-1-methyl-6-phenylimidazo[4,5-b]pyridine (PhIP), N-acetyldopamine dimers, allopurinol, anetholdithiolthione, apocynin, 5,6,3′,5′-tetramethoxy 7,4′-hydroxyflavone, astaxanthin, avenanthramides, benidipine, bis-eugenol, butylated hydroxyanisole (BHA), cepharanthine, 3,4-dihydroxycinnamic acid, carnosol, β-carotene, carvedilol, catechol derivatives, chalcone, chlorogenic acid, 5-chloroacetyl-2-amnio-1,3-selenazoles, cholestin, chroman-2-carboxylic acid N-substituted phenylamides, 3-methyl-1,2-cyclopentanedione, diferulolylmethane, dimethoxycurcumin, EF24 analog, dehydroepiandrosterone (DHEA), DHEA-sulfate, dibenzylbutyrolactone lignans, diethyldithiocarbamate, diferoxamine, dihydroisoeugenol, isoeugenol, epoxypseudoisoeugenol-2-methyl butyrate, dihydrolipoic acid, dimethyldithiocarbamates, dimethylsulfoxide, disulfuram, ebselen, edaravone, EPC-K1 (phosphodiester compound of vitamin E and vitamin C), epigallocatechin-3-gallate, ergothioneine, ethyl pyruvate, ethylene glycol tetraacetic acid, eupatilin, fisetin, xanthohumol, genistein, kaempferol, quercetin, daidzein, flavone, isorhamnetin, naringenin, pelargonidin, finestin, folic acid, γ-glutamylcysteine synthetase, garcinol, glutathione, 2-methoxyphenol, hematein, hinokitiol, hydroquinone, 23-hydroxyursolic acid, IRFI 042, iron tetrakis, isosteviol, isovitexin, isoliquiritigenin, kallistatin, L-cysteine, lacidipine, lazaroids, lupeol, lutein, magnolol, maltol, manganese superoxide dismutase (Mn-SOD), melatonin, 21 (α, β)-methylmelianodiol, N-acetyl-L-cysteine, nacyselyn, nordihydroguaiaritic acid, ochnaflavone, 2,3-dihydro-3,5-dihydroxy-6-methyl-4H-pyranone, orthophenanthroline, N-(3-oxo-dodecanoyl) homoserine lactone, paricalcitol, hydroquinone and tert-butyl hydroquinone, α-phenyl-n-tert-butyl-nitrone, phenylarsine oxide, pitavastatin, prodelphinidin B2 3, 3′ di-O-gallate, pterostilbene, pyrrolinedithiocarbamate, quercetin, redox factor 1, rotenone, roxithromycin, rutin, S-allyl-cysteine, salogaviolide, sauchinone, silybin, spironolactone, taxifolin, tempol, 5-(4-chlorophenyl)-N-hydroxy-(4-methoxyphenyl)-N-methyl-1H-pyrazole-3-propanamide, thio avarol derivatives, thymoquinone, tocotrienol, UDN glycoprotein, 2-hydroxy-3-methoxybenzaldehyde), vitamin C, vitamin B6, vitamin E and derivatives, α-torphryl succinate, α-torphryl acetate, 2,2,5,7,8-pentamethyl-6-hydroxychromane, and yakuchinone A and B.

A variety of proteasome and protease inhibitors can be used as NF-κB signaling pathway inhibitors. Non-limiting examples of such proteasome and protease inhibitors include: N-acetyl-leucinyl-leucynil-norleucynal, N-acetyl-leucinyl-leucynil-methional, carbobenzoxyl-leucinyl-leucynil-norvalinal, carbobenzoxyl-leucinyl-leucynil-leucynal, lactacystine, beta-lactone, boronic acid peptide, dithiocarbamate complexes with metals, ubiquitin ligase inhibitors, bortezomib, salinosporamide A, cyclosporin A, FK506, Tacrolimus), deoxyspergualin, disulfuram, PT-110, N-acetyl-DL-phenylalanine-β-naphthylester, N-benzoyl L-tyrosine-ethylester, 3,4-dichloroisocoumarin, diisopropyl fluorophosphates, N-α-tosyl-L-phenylalanine chloromethyl ketone, and N-α-tosyl-L-lysine chloromethyl ketone.

In addition, IκBα phosphorylation and degradation inhibitors can also be used as NF-κB signaling pathway inhibitors. Non-limiting examples of such IκBα phosphorylation and degradation inhibitors include: desloratadine; diphenhydramine, bikunin, Ron tyrosine kinase inhibitor, TAK-242, salmeterol, CPU0213, doxazosin alpha1-adrenergic receptor antagonist, anti-CD146 antibody AA98, calagualine (fern derivative), NS3/4A, golli BG21, NPM-ALK oncoprotein, NS5A, LY29, LY30, Shiga toxin, evodiamine, rituximab, kinase suppressor of ras (KSR2), cholecystokinin ocatpeptide (CCK-8), M2L, pefabloc, rocaglamides, Ymer, Epoxyquinol B, Betaine, TNAP, desflurane, geldanamycin, Laretia acaulis azorellane diterpenoids, MC160, NS5B, tetrandine, 4(2′-Aminoethyl)amino-1,8-dimethylimidazo[1,2-a]quinoxaline and 4-amino derivatives thereof, 1-O-acetylbritannilactone, 2-amino-3-cyano-4-aryl-6-(2-hydroxy-phenyl)pyridine derivatives, acrolein, anandamide, AS602868, cobrotoxin, Core protein (Hepatitis C), 1-[2-cyano-3,12-dioxooleana-1,9(11)-dien-28-oyl]imidazole, dihydroxyphenylethanol, herbimycin A, Inhibitor 22, isorhapontigenin, manumycin A, 6-methyl-2-propolyimino-6,7-dihydro-5H-benzo[1,3]oxathiol-4-one, MLB120, 6-methoxycomaparvin, 6-methooxycomaparvin 5-methyl ether, vIRF3 (KSHV), nitric oxide, SC-514, thienopyridine, acetyl-boswellic acids, BMS-345541, butein, β-carboline, CYL-19s, CYL-26z, 2-amino-6-[2-(cyclopropylmethoxy)-6-hydroxyphenyl]-4-piperidin-4-yl nicotinonitrile, berberine, flavopiridol, cyclopentones, dehydroascorbic acid, M protein (SARS-Cornonavirus protein), IMD-0354, Jesterone dimer, KINK-1, LCY-2-CHO, prolyl hydroxylase-1, naphthopyrones, neuropeptides CGRP, PACAP, and VIP, PS-1145, 2-[(aminocarbonyl)amino]-5-(4-fluorophenyl)-3-thiophenecarboxamides, 1′-acetoxychavicol acetate, 17-acetoxyjolkinolide B, 6-nonadecyl-salicylic acid, apigenin, asiatic acid, cardamomin, CDDO-Me (synthetic triterpenoid), CHS 828 (anticancer drug), CML-1, compound 5 (Uredio-thiophenecarboxamide derivative), CT20126, 3,4-dihydroxybenzalacetone, diosgenin, E3-14.7K (Adenovirus), E3-10.4K/14.5K (Adenovirus), E7 (human papillomavirus), furonaphthoquinone, guggulsterone, heparin-binding epidermal growth factor-like growth factor, falcarindol, hepatocyte growth factor, honokiol, humulone, hypoestoxide, indolecarboxamide derivative, labdane diterpenoids, LF15-0195, γ-mangostin, garcinone B, (amino)imidazolylcarboxaldehyde derivative, imidazolylquinoline-carboxaldehyde derivative, kahweol, kava (Piper methysticum) derivatives, menatetrenone, metformin, ML120B, 3,5,7,2′,4′-pentahydroxyflavone, morusin, MX781, N-acetylcysteine, nitrosylcobalamin, NSAIDs, hepatitis C virus NS5B, PAN1, pectin, pinitol, PMX464, pyrazolo[4,3-c]quinolone derivative, pyridooxazinone derivative, N-(4-hydroxyphenyl) retinamide, scytonemin, SPC-839, sulforaphane, phenylisothiocyanate, survanta, Torque Teno virus ORF2, piceatannol, 5-hydroxy-2-methyl-1,4-naphthoquinone, NEMO CC2-LZ peptide, AGRO100 (G-quadraplex oligodeoxynucleotide), PTEN, theaflavin, tilianin, withanolides, zerumbone, silibinin sulfasalazine and analogs thereof, quercetin, rosmarinic acid, staurosporine, γ-tocotrienol wedelolactone, betulinic acid, ursolic acid, thalidomide and analogs thereof, Fas-associated factor-1, Interleukin-10, MC160 (molluscum contagiosum virus), monochloramine, glycine chloramine GS143, Salmonella Secreted Factor L, anethole, anti-thrombin III, sodium salicylate, azidothymidine, baoganning, E3((4-methylphenyl)-sulfonyl)-2-propenenitrile, E3((4-t-butylphenyl)-sulfonyl)-2-propenenitrile, benzyl isothiocyanate, cyanidin 3-O-glucoside, cyanidin 3-O-(2(G)-xylosylrutinoside, cyanidin 3-O-rutinoside, buddlejasaponin IV, cacospongionolide B, calagualine, carboplatin, cardamonin, chorionic gonadotropin, cordycepin, 1-hydroxy-2-hydroxymethyl-3-pent-1-enylbenzene, decursin, delphinidin, dexanabinol, digitoxin, diterpenes, docosahexaenoic acid, extensively oxidized low density lipoprotein, 4-hydroxynonenal, FBD, fragile histidine triad protein, Fructus Ligustrum lucidi, gabexate mesilate, [6]-gingerol, imatanib, guggulsterone, 4-hydroxy-3,6,7,8,3′,4′-hexamethoxyflavone, hydroquinone, ibuprofen, indirubin-3′-oxime, interferon-α, isobutyl nitrite, kaempferol, kushen flavonoids, kurarinone, melatonin, methotrexate, monochloramine, nafamostat mesilate obovatol, oleandrin, oleanolic acid, omega-3 fatty acids, panduratin A, petrosaspongiolide M, pinosylvin, inositol hexakisphosphate, prostaglandin A1, 20(S)-protopanaxatriol, rengyolone, rottlerin, saikosaponin-d, 13-methyl-[1,3]-benzodioxolo-[5,6-c]-1,3-dioxolo-4,5 phenanthridinium, scoparone, sesaminol glucosides, shikonins, silymarin, SOCS1, spilanthol, statins, sulindac, 1-naphthylethyl-6,7-dihydroxy-1,2,3,4-tetrahydroisoquinoline, 1,2,4-thiadiazolidine derivatives, tomatidine, vesnarinone, xanthoangelol D, YC-1, YopJ (Yersinia pseudotuberculosis), acetaminophen, alachlor, allylpyrocatechol, α-melanocyte-stimulating hormone, amentoflavone, L-ascorbic acid, aucubin, baicalein, N-(quinolin-8-yl)benzenesulfonamindes, β-lapachone, 1-bromopropane, buchang-tang, 8-methyl-N-vanillyl-6-nonenamide, catalposide, clomipramine/imipramine, cyclolinteinone, DA-9601, diamide (tyrosine phosphatase inhibitor), dihydroarteanniun, dobutamine, docosahexaenoic acid, E-73 (cycloheximide analog), ecabet sodium, 3-methyl-1,6,8-trihydroxyanthraquinone, equol, erbstatin, estrogen (E2), ethacrynic acid, fludarabine, fosfomycin, gabexate mesilate, gamisanghyulyunbueum, genistein, genipin, glabridin, ginsenoside Re, glimepiride, glucosamine sulfate, glucosamine carboxybutyrylate), γ-glutamylcysteine synthetase, glutamine, glycochenodeoxycholate, gumiganghwaltang, gum mastic, hypochlorite, IL-13, incensole acetate, isomallotochromanol, isomallotochromene, K1L (Vaccinia virus protein), leflunomide metabolite (A77 1726), lidocaine, lipoxin A4, losartan, 2-(4-morpholinyl)-8-phenylchromone, MC159 (Molluscum contagiosum virus), meloxicam, 5′-methylthioadenosine, midazolam, momordin I, neurofibromatosis-2 protein, pectenotoxin-2, penetratin, pervanadate, phenylarsine oxide, β-phenylethyl, 8-methylsulphinyloctyl isothiocyanates (MSO), phenyloin, c-phycocyanin, polymyxin B, pituitary adenylate cyclase-activating polypeptide, prostaglandin 15-deoxy-δ(12,14)-PGJ(2), prodigiosin, PS-341, resiniferatoxin, sabaeksan, SAIF, San-Huang-Xie-Xin-Tang, scutellarin, parthenolide, ergolide, alpha-humulene, trans-caryophyllene, sevoflurane/isoflurane, siegeskaurolic acid, ST2, taurene bromamine, thiopental, tipifarnib, TNP-470, tussilagone, U0126, ursodeoxycholic acid, zinc, Molluscum contagiosum virus MC159 protein, vasoactive intestinal peptide, HIV-1 Vpu protein, TrCP, epoxyquinone A monomer, and Ro106-9920. The NF-κB signaling pathway inhibitors described herein include kinase inhibitors (e.g., inhibitors of cellular receptor tyrosine kinases that are upstream of NF-κB activation, as well as inhibitors of cytosolic kinases that are upstream of NF-κB activation), inhibitors that block NF-κB nuclear translocation or increase NF-κB nuclear export, inhibitors that block NF-κB from binding to promoter elements in NF-κB target genes, or inhibitors that block NF-κB transactivation of a target gene.

Additional examples of NF-κB signaling pathway inhibitors include: conophylline, MOL 294, pigment epithelium derived factor, perrilyl alcohol, MAST205, rhein, 15-deoxy-prostaglandin J(2), palmitoylethanolamide, 4′,5,7-trihydroxyflavone, aresenic trioxide, surfactant protein A, DQ 65-79 (amino acids 65-79 of the alpha helix of the alpha-chain of the class II HLA molecule DQA03011), cysteamine, dexamethasone, prednisone, methylprednisolone, IL-13, IL-11, alpha-pinene, NEF (HIV-1), R-etodolac, SR141716, vitamin D, Fox1j, NSP1 (rotavirus), dioxin, alginic acid, apilimod, astragaloside IV, atorvastatin, azacitidine, N(1)-Benzyl-4-methylbenzene-1,2-diamine), carbaryl, captopril, carnostic acid, celastrol, α-chaconine, chiisanoside, CHS 828, corilagin, CP-1158, dehydroxymethylepoxyquinomicin, 15-deoxyspergualin, dipyridamole, disulfuram, diltiazem, ERB-041, eriocalyxin B, eutigoside C, FAK-related nonkinase, florfenicol, β-funaltrexamine, gangliosides, glucorticoid-induced leucine zipper protein (GILZ), HDAC inhibitors, sodium valproate, MS-275, hirsutenone, indole-3-carbinol, JM34 (benzamide derivative), 4-Methyl-(3-phenyl-propyl)-benzene-1,2-diamine, 6-Hydroxy-7-methoxychroman-2-carboxylic acid phenylamide, leptomycin B, levamisole, lidamycin, 2-(4-morpholynl) ethyl butyrate hydrochloride, methimazole, MNF (IkB-like Myxoma virus), montelukast, NSA9 (prothrombin fragment), 2′,8″-biapigenin, glimperide, nucling, O,O′-bismyristoyl thiamine disulfide (BMT), oregonin, 1,2,3,4,6-penta-O-galloyl-beta-d-glucose, pergolide, phallacidin, pimecrolimus, piperine, pitavastatin, PN-50, POP2, pravastatin, propofol, RelA peptides P1, RelA peptide P6, retinoic acid receptor-related orphan receptor-alpha, rolipram, SC236, selenomethionine, sorafenib, cornuside, sopoongsan, sphondin (furanocoumarin derivative from Heracleum laciniatum), TAT-SR-IkBa; MTS-SR-IkBa, thymulin, ZUD protein, ZAS3 protein, clarithromycin, fluvastatin, leflunomide, VGX-1027, oxidized 1-palmitoyl-2-arachidonoyl-sn-glycero-3-phosphorylcholine, 3C protease (Poliovirus), 5F (from Pteri syeminpinnata L), serratamolide, azithromycin, cantharidin, gamboic acid, meiduoluomi, neomycin, omapatrilat, enalapril, CGS 25462, onconase, paeoniflorin, pouterin, rapamycin, shenfu, triflusal, zoledronic acid, hepatoma protein, andrographolide covalent adduct with Cys-62 of p50, selencotyanates covalent adduct with Cys-62 of p50, melittin, ethyl pyruvate, 25-acetylcimigenol xylpoyranoside, 1′-acetoxychavicol acetate, 2-acetylaminofluorene, actinodaphine, adiponectin, nicotinamide, albaconol, 7-amino-4-methylcoumarin, amrinone, angiopoietin-1, anthocyanins, (S)-armepavine, artemisinin, artepillin C, artesunate, arzanol, atrial natriuretic peptide, atrovastat, AvrA protein, 5,6,7-trihydroxyflavone, benfotiamine, β-catenin, β-lapachone, biliverdin, bisphenol A, bromelain, calcium/calmodulin-dependent kinase kinase (CaMKK), ionomycin, UTP, thapsigargin, calcitriol, camptothecin, candesartan, cannabidiol, caprofen, capsiate, carbocisteine, catalposide, CDX2, celecoxib, germcitabine, cheongyeolsaseuptang, chitosan, chitooligosaccharides, cinnamaldehyde, 2-methoxycinnamaldehyde, 2-hydroxycinnamaldehyde, guaianolide 8-deoxylactucin, chlorophyllin chondrotin sulfate, clarithromycin, cloricromene, CORM-2, cocaethylene, 6-hydroxy-7-methoxychroman-2-carboxylic acid phenylamide, cryptotanshinone, cyanoguanidine CHS 828, cytochalasin D,DA-9201, salvionic acid B, κB site decoy oligonucleotides, deoxypodophyllotoxin, diallyl trisulfide, diamide, diarylheptanoid 7-(4′-hydroxy-3′-methoxyphenyl)-1-phenylhept-4-en-3-one, α-difluoromethylomithine, S-diclofenac (2-[92,6-dichlorphenyl-amino]benzeneacetic acid 4-[3H-1,2,dithiol-3-thione-5-y) pheny ester), 7,7′-dihydroxy bursehernin, 3,3′-diindolymethane, DIM/13C, dimemorfan, docetaxel, dohongsamultang, 4,10-dichloropyrido[5,6:4,5]thieno[3,2-d′:3,2-d]-1,2,3-ditriazine, E1B (Adenovirus), E3330 (quinone derivative), ellagic acid, ent-kaurane diterpenoids, epinastine hydrochloride, epoxyquinol A, ergosterol peroxide, ergosterol, cervisterol, erythromycin, evodiamine, fenoldopam, fexofenadine hydrochloride, fentanyl, fibrates, FK778, FLICE-Like Inhibitory Protein (FLIP), flunixin meglumine, flurbiprofen, flutamide, fMLP (bacterial peptide), folimycin, fucoidan, G-120 (Ulmus davidiana Nakai glycoprotein), gallic acid, ganoderic acid, garcinol, Gax (homeobox protein), geranylgeraniol, geranylgeranylacetone, ghrelin, gigantol, gingyo-san, ginkgolide B, ginseng saponin 20-O-b-D-glucopyranosyl-20(S)-protopanaxadiol, glycyrrhizin, H4/N5 (IkB-like proteins of Microplitis demolitor bracovirus), halofuginone, helenalin, hematein, heparin, hesperetin, hyaluronan, hydroxyethyl starch, hydroxyethylpuerarin, hypericin, hydroquinone, ICP27 (HSV-1), interleukin-4, IkB-like protein A238L, ilimaquinone, indoxam, insulin-like growth factor binding protein-3, irisilidone, N1-benzyl-4-methylbenzene-1,2-diamine), kamebakaurin, Kaposi's sarcoma-associated herpesvirus K1 protein, ketamine, KT-90 (morphine synthetic derivative), lichochalcone E, lignin, linoleic acid, lovastatin, lornoxicam, lucidenic acid, malloapelta B, mercaptopyrazine, 2-methoxyestradiol, 6-(methylsulfinyl)hexyl isothiocyanate, mevinolin, 5′-methylthioadenosine (MTA), methylglyoxal, mirtazapine, monomethylfumarate, morphine, moutan cortex, moxifloxacin, myricetin, NDPP1 (CARD protein), N-ethyl-maleimide (NEM), naringen, nicorandil, nicotine, nifedipine, nitric oxide-donating aspirin, nilvadipine, nitrosoglutathione, NS1 (Influenza A), NS3/4A (Hepatitis C virus), oleic acid, orazipone, oridonin, 1,2,3,4,6-penta-O-galloyl-beta-D-glucose, p202a (interferon inducible protein), p21, panepoxydone, 1-(5′-oxohexyl) 3,7-dimethylxanthine, peptide YY, pepluanone, perindopril, 6(5H)-phenanthridinone, benzamide, phylligenin, PIAS1 (protein inhibitor of activated STAT1), pioglitazone, pirfenidone, polyozellin, pregabalin, prenylbisabolane 3, pro-opiomelanocortin, procyandin B2, prostaglandin E2, protein-bound polysaccharide (PSK), PYPAF1 protein, pyridine N-oxide derivatives, pyrithione, pyrrole-imidazole polyamides, quinadril, quinic acid, Raf Kinase Inhibitor Protein (RKIP), rapomycin, raloxifene, raxofelast, rebamipide, ribavirin, rifamides, ritonavir, rosiglitazone, roxithromycin, sanggenon C Santonin diacetoxy acetal derivative, Scapharca broughtonii factor, secretory leukoprotease inhibitor (SLPI), N-(p-coumaroyl) serotonin, sesamin, Shen-Fu, sodium butyrate, sodium phenylbutyrate, sodium phenylacetate, Siah2, silibinin, simvastatin, sinomenine, Siva-1, SM-7368, sodium tanshinone IIA sulfonate, solanum nigrum L 150 kDa glycoprotein, SP100030, suberosin, sulfasalazine, SUN C8079, surfactant protein A, (+) syringaresinal-di-O-beta-d-glucoside, T-614, talosin A, temozolomide, tetramethylpyrazine, thiazolidinedione MCC-555, trichostatin A, triptolide, tyrphostin AG-126, UNBS1450 01, uranyl acetate, ursolic acid, uteroglobin, V,C proteins (Sendai virus), vascular endothelial growth factor, verapamil, withaferin A, 5,7-dihydroxy-8-methoxyflavone, xanthohumol, xanthorrhizol, xylitol, YC-1, zapotin, zyflamend, 8-acetoxy-5-hydroxyumbelliprenin, APC0576, artemisolide, BZLF1 (EBV protein), chromene derivatives, D609 (phosphatidylcholine-phospholipase C inhibitor), dehydroevodiamine, 4′-demethyl-6-methoxypodophyllotoxin, 5-alpha-dihydrotestosterone, ethyl 2-[(3-methyl-2,5-dioxo(3-pyrrolinyl))amino]-4-(trifluoromethyl)pyrimidine-5-carboxylate, CHFR, cycloprodigiosin hycrochloride, DC-81, dimethylfumarate, E1A (Adenovirus), eckol/dieckol, FAD24, gallotannins, dexametasone, prednisone, methylprednisolone, glycodelin-A, gypenoside XLIX, hesperidin, 4-hydroxykobusin, IE2p86 (CMV), N-aminoimidazole derivatives (NR-818), Lys-Pro-Val tripeptide, LZAP, nobiletin, NRF (NF-kB repression factor), paeonol, phenethylisothiocyanate, 4-phenylcoumarins, phomol, PIAS3, pranlukast, psychosine, pycnogenol, quinazolines, resveratrol, RO31-8220, saucerneol D, saucerneol E, SB203580, streptochlorin, N-(3,4-dimethoxycinnamoyl)anthranilic acid, 3,4,5-trimethoxy-4′-fluorochalcone, LY294,002, mesalamine, mesuol, trastuzumab, rituximab, PTX-B, 9-aminoacridine (9AA) derivatives (e.g., quinacrine), SK-126, adenosine, cyclic AMP, 17-allylamino-17-demethoxygeldanamycin, 6-aminoquinazoline derivatives, luteolin p65, manassantins A and B, MYBBPla p65, sphingosine-1-phosphate, telithromycin, tetracyclic A, tetrathiomolybdate, trilinolein, troglitazone, valerenic acid/acetylvalerenolic acid, wortmannin, α-zearalenol, antithrombin (III), rifampicin, and mangiferin. The examples of NF-κB signaling pathway inhibitors described herein are not limiting and additional NF-κB signaling pathway inhibitors are known in the art.

### VIII. Methods of Treatment

The compounds described herein, i.e., mitochondrial-targeted chaperone inhibitors, death receptor agonists, autophagy inhibitors, and NF-κB signaling pathway inhibitors are useful in the treatment of disorders associated with uncontrolled cellular proliferation, as occurs, for example, in tumor formation and in cancer. In some embodiments, tumors treated by a method described herein can be associated with a cancer described herein.

Generally, the methods include administering a therapeutically effective amount of at least one mitochondrial-targeted chaperone inhibitor and at least one death receptor agonist, autophagy inhibitor, and NF-κB signaling pathway inhibitor, to a subject who is in need of, or who has been determined to be in need of, such treatment.

As used in this context, to “treat” means to ameliorate at least one symptom of the disorder associated with uncontrolled cellular proliferation. Ideally, a treatment can result in the death of the proliferating cells, or in a decrease in the rate of proliferation of the cells (i.e., the cancer or tumor cells).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected.

The compositions described herein can be administered systemically, locally, or both, using methods known in the art, e.g., parenteral, oral, mucosal, or other routes of administration. As one of skill in the art will appreciate, the route of administration should be selected based on suitability for the treatment of the specific condition, and the formulation of the composition.

The compositions described herein can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The compositions described herein are useful in the treatment of tumors and cancer. The compositions described herein can be administered to a patient diagnosed with cancer, e.g., any of the types of cancers referred to herein. For example, the mitochondrial-targeted chaperone inhibitor and/or death receptor agonist, autophagy inhibitor, and/or NF-κB signaling pathway inhibitor disclosed herein can be used, without limitation, to treat a subject suffering from one or more of a cancer or tumor of the lung, breast, epithelium, large bowel, rectum, testicle, gallbladder, bile duct, biliary tract, prostate, colon, stomach, esophagus, pancreas, liver, uterus, ovary, or brain. In certain embodiments, the compositions described herein are useful in the treatment of chronic myelogenoeous leukemia, B lymphoblastoid leukemia, breast adenocarcinoma, lung adenocarcinoma, prostate adenocarcinoma, glioblastoma (e.g., glioblastoma multiforme), colorectal adenocarcinoma, and cervical carcinoma. In other examples, a mitochondrial-targeted chaperone inhibitor and/or death receptor agonist, autophagy inhibitor, and/or NF-κB signaling pathway inhibitor disclosed herein can be used to treat, without limitation, a subject suffering from haemangioma, Hodgkin's disease, non-Hodgkin's lymphoma (e.g., large cell non-Hodgkin's lymphoma), multiple myeloma, malignant lymphoma, leukemia, polycythemia vera, neuroblastoma, retinoblastoma, myelodysplastic syndrome with refractory anemia, neuroblastoma, glioma, pheochromocytoma, soft tissue sarcoma, maxillary cancer, lingual cancer, lip cancer, mouth cancer, melanoma, or non-melanoma skin cancer (e.g., squamous cell carcinoma). In general, cancers that can be treated by the compositions and candidate compounds described herein include but are not limited to carcinomas, sarcomas, lymphomas, leukemias, or germ cell tumors. In preferred embodiments, the compositions described herein can be administered to a patient diagnosed with cervical cancer, breast cancer, prostate cancer, lung cancer (e.g., small-cell lung cancer or non-small cell lung cancer), non-Hodgkin's lymphoma, multiple myeloma, epithelial carcinoma, colorectal cancer, Burkitt lymphoma, myeloid leukemia, leukemic monocyte lymphoma, myeloblastic leukemia, and B cell lymphoma.

Administration and Dosing

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of a protein, polypeptide, antibody, or other compound can include a single treatment or, preferably, can include a series of treatments.

If the compound is a small molecule, exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Identifying Subjects for Treatment

In some embodiments, the methods include (i) identifying and selecting an individual suffering from cancer, and optionally (ii) determining if the individual's cancer cells express (e.g., express high levels) of Hsp90 chaperones in the mitochondria and/or one or more death receptors. If these cells express Hsp90 chaperones in the mitochondria and/or one or more death receptors, then the individual is a candidate for, i.e., can be selected for, treatment with a mitochondrial-targeted chaperone inhibitor and a death receptor agonist, and the method further includes (iii) administering to the individual a pharmaceutical composition including at least one mitochondrial-targeted chaperone inhibitor and a pharmaceutical composition including at least one death receptor agonist, an autophagy inhibitor, and NF-κB signaling pathway inhibitor.

Individuals with cancer can be identified using methods known in the art, e.g., because they display symptoms or as a result of screening. Additional clinical tests can be performed and include, but are not limited to, blood tests, X-rays, CT scans, endoscopy, and histological examination of biopsy tissue, to confirm the diagnosis.

Symptoms of cancer in an individual include, but are not limited to, unusual lumps or swelling, hemorrhage, pain and/or ulceration, enlarged lymph nodes, cough and hemoptysis, hepatomegaly (enlarged liver), bone pain, fracture of affected bones and neurological symptoms, weight loss, poor appetite and cachexia (muscle wasting), excessive sweating, and anemia.

Screens for identifying individuals with cancer are known in the art. Screening methods include, but are not limited to, self-examination, mammograms, fetal occult blood testing, cervical cytology (e.g., Pap smear), digital rectal exam, prostate specific antigen (PSA) blood testing, sigmoidoscopy, which looks for visual abnormality in the rectum and lower part of the colon, and colonoscopy, which allows visualization of the rectum and entire colon, and double contrast barium enema (DCBE), which allows radiographic examination of the rectum and colon.

A number of methods are known in the art for detecting expression of chaperones in the mitochondria and/or expression of a death receptor, including immunoassays, e.g., using an antibody. For example, the detection of chaperones in mitochondria can be achieved by obtaining mitochondrial and submitochondrial fractions, followed by the use of known detection methods, such as Western blotting, immunoelectron microscopy with an antibody to Hsp90, and matrix-assisted laser desorption/ionization (MALDI) proteomics (e.g., mass spectroscopy and time-of-flight analysis) of mitochondrial fractions. The detection of a death receptor can be achieved, e.g., by obtaining a membrane fraction, followed by the use of known detection methods, such as Western blotting, immunoelectron microscopy with an antibody to Hsp90, and matrix-assisted laser desorption/ionization (MALDI) proteomics (e.g., mass spectroscopy and time-of-flight analysis).

Additional methods of identifying individuals who are candidates for treatment with a chaperone inhibitor and a death receptor agonist are disclosed herein. In these methods, a cancer cell from an individual is (i) exposed to a mitochondrial-targeted chaperone inhibitor and/or a death receptor agonist, an autophagy inhibitor, and/or a NF-κB signaling pathway inhibitor and (ii) assayed for the presence of one or more of the following activities: increased cell death, loss of cell viability, loss of mitochondrial membrane potential, loss of mitochondrial membrane integrity (e.g., Smad or cytochrome c release), and loss of Hsp90 chaperone activity (e.g., degradation of Akt kinase). Methods for performing such assays are known in the art and include flow cytometry, the MTT assay, gel electrophoresis, and western blotting. Exemplary methods are also described in the Examples herein.

If the cancer cell exhibits one or more of these activities, then the individual is classified as a candidate for treatment with at least one mitochondrial-targeted chaperone inhibitor and at least one death receptor agonist, autophagy inhibitor, and NF-κB signaling pathway inhibitor. In other new methods, cancer cells from the same individual are placed in culture media. Some of the cancer cells are contacted with at least one mitochondrial-targeted chaperone inhibitor and at least one death receptor agonist, autophagy inhibitor, and NF-κB signaling pathway inhibitor, and cultured under conditions that allow the cells to proliferate. If the at least one mitochondrial-targeted chaperone inhibitor and the at least one death receptor agonist, autophagy inhibitor, and NF-κB signaling pathway inhibitor inhibit proliferation and/or induce apoptosis of the contacted cancer cells, e.g., relative to cells that are not contacted with the agents, then the individual is a candidate for treatment with at least one mitochondrial-targeted chaperone inhibitor and at least one death receptor agonist, autophagy inhibitor, and NF-κB signaling pathway inhibitor.

Methods of Treating Therapeutic-Resistant Cancer

Also provided are methods of treating a therapeutic-resistant cancer in a subject. These methods require the administering to a subject having cancer cells resistant to a cancer therapeutic at least one mitochondrial-targeted chaperone inhibitor and the cancer therapeutic, where the at least one mitochondrial-targeted chaperone inhibitor and the cancer therapeutic are administered in amount sufficient to treat the therapeutic-resistant cancer. In some embodiments, these methods further include identifying a subject as having a therapeutic-resistant cancer. In some embodiments, the subject is identified as having a therapeutic-resistant cancer by detecting or measuring an increase in NF-κB signaling pathway activity in a cancer cell in the subject. A subject having a therapeutic-resistant cancer can be also be identified by a skilled health care professional using a number of methods described herein. For example, a subject that has a therapeutic-resistant cancer may be identified by a decreased or no detectable response to a cancer therapeutic (e.g., a cancer therapeutic that works well (e.g., enhances cancer cell apoptosis in a cancer derived from a similar tissue in another patient). The co-administration of at least one mitochondrial-targeted chaperone inhibitor and the cancer therapeutic increases the therapeutic effect (e.g., enhances apoptosis in cancer cells resistant to a cancer therapeutic) compared the therapeutic effect observed upon administration of the cancer therapeutic alone.

By the term “cancer therapeutic” is meant any cancer therapy known in the art. Non-limiting examples of cancer therapy include without limitation: chemotherapeutic agents and biologics (e.g., Aprepitant, Cytoxan®, Emend®, Gemzar®, Taxotere®, zoledronic acid, Zometa®, Efudex®, Vidaza®, Temodar®, Abraxane®, Aromasin®, Anastrozole, Capecitabine, Doxil®, Faslodex®, Femara®, Ixabepilone, Ixempra™, Letrozole, Taxotere®, Trastuzumab, Tykerb®, Xeloda®, Zoledronic acid, Avastin®, Bevacizumab, Erbitux®, Gleevec®, Sutent®, Torisel™, Nexavar®, Sutent®, arsenic trioxide, Campath®, Cytoxan®, Alimta®, Tarceva®, Rituxan®, Velcade®, Doxil®, lenalidomide, Revlimid®, and Thalomid®. Additional cancer therapeutics are known in the art.

### VIII. Pharmaceutical Compositions

The mitochondrial-targeted chaperone inhibitors, death receptor agonists, autophagy inhibitors, and NF-κB signaling pathway inhibitors described herein (all of which can be referred to herein as “active compounds”) can be incorporated into pharmaceutical compositions. Such compositions typically include one or more active compounds and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions. Also included are the pharmaceutical compositions themselves, and pharmaceutically acceptable salts of the compounds described herein. It is well known in the pharmacological arts that nontoxic addition salts of pharmacologically active amine compounds do not differ in activities from their free base.

Pharmaceutically acceptable salts include both acid and base addition salts. “Pharmaceutically acceptable salt” refers to those salts which retain the biological effectiveness and properties of the free bases and which are not biologically or otherwise undesirable. Suitable pharmaceutically acceptable acid addition salts can be formed with inorganic acids such as hydrochloric acid, hydrobromic acid, sulfuric acid, nitric acid, phosphoric acid, and the like, and organic acids such as acetic acid, propionic acid, glycolic acid, pyruvic acid, fumaric acid, tartaric acid, citric acid, benzoic acid, cinnamic acid, mandelic acid, methanesulfonic acid, and p-toluenesulfonic acid, and the like. Pharmaceutically acceptable base addition salts include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic non-toxic bases include salts of primary, secondary, and tertiary amines, substituted amines, including naturally occurring substituted amines, cyclic amines and basic ion exchange resins, such as isopropylamine, tripropylamine, ethanolamine, 2-diethylaminoethanol, 2-dimethylaminoethanol, dicyclohexylamine, lysine, arginine, histidine, caffeine, procain, hydrabamine, choline, betaine, ethylenediamine, glucosamine, methylglucamine, theobromine, purines, piperazines, piperidine, polyamine resins and the like. Particularly preferred organic non-toxic bases are isopropylamine, diethylamine, ethanol-amine and dicyclohexylamine.

A pharmaceutical composition is generally formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate or gelatin, in the composition.

Sterile injectable solutions can be prepared by incorporating an active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

For administration by inhalation, a compounds is delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Although applicants do not wish to be bound by theory, any non-specific cytotoxic effects of a systemically administered mitochondrial-targeted chaperone inhibitor as described herein are expected to be minimal, for at least the following reasons: levels of mitochondrial Hsp90 and TRAP 1 are low in most normal tissue; as demonstrated herein, mitochondrial localization of Hsp90 and TRAP-1 is generally tumor cell-specific, so that the inhibitors will preferentially accumulate in the mitochondria of tumor cells; in those normal tissues that have mitochondrial-localized Hsp90 and TRAP-1, the activity of Hsp90 and TRAP-1 is decreased relative to the activity in tumor cells; and the blood-brain barrier is expected to protect the brain.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Nucleic acid molecules encoding a polypeptide described herein can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen et al., Proc. Natl. Acad. Sci. USA 1994, 91:3054-57). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Modifications such as lipidation can be used to stabilize proteins and to enhance uptake and tissue penetration. A method for lipidation is described by Cruikshank et al., 1997, J. Acquir. Immune Defic. Syndr. Hum. Retrovirol., 14:193-203.

The pharmaceutical compositions can be included in a pharmaceutical pack or kit comprising one or more containers filled with one or more of the ingredients of the pharmaceutical compositions of the invention. Optionally associated with such container(s) can be instructions to use the pharmaceutical compositions to enhance apoptosis, to induce cancer or tumor cell death, or to treat a proliferative disorder, e.g., a cancer (e.g., a cancer described herein). Optionally associated with such container(s) can be a notice in the form prescribed by a governmental agency regulating the manufacture, use or sale of pharmaceuticals or biological products, which notice reflects approval by the agency of manufacture, use or sale for human administration.

### IX. Nucleic Acids

Also included within the present disclosure are nucleic acids that encode peptide-based mitochondrial-targeted chaperone inhibitors and/or death receptor agonists, autophagy inhibitors, and/or NF-κB signaling pathway inhibitors as described herein.

Nucleic acids that are used in the methods this invention can encode a death receptor agonist, an autophagy inhibitor, an NF-κB signaling pathway inhibitor, or any of the peptides identified by the methods disclosed herein that bind to and inhibit mitochondrial Hsp90 chaperones, e.g., Hsp90 and TRAP-1. The nucleic acids disclosed herein also include nucleic acids encoding modified versions of peptides that bind to and inhibit mitochondrial Hsp90 chaperones, e.g., retro peptides, peptides linked to a heterologous polypeptide sequence, peptides linked to a mitochondrial-penetrating sequence, peptides linked to a cellular internalization sequence, and retro peptides linked to a mitochondrial-penetrating sequence.

The nucleic acids disclosed herein also include nucleic acids encoding modified versions of death receptor agonists, e.g., polypeptide and antibody agonists. In some embodiments, the nucleic acid can include a signal sequence that directs expression of the encoded polypeptide to the cell membrane and/or cleavage of the encoded polypeptide to produce a soluble form.

In some embodiments, the nucleic acids encode mitochondrial-targeted chaperone inhibitors and/or death receptor agonists, autophagy inhibitors, and/or NF-κB signaling pathway inhibitors for use in gene therapy.

Nucleic acids disclosed herein include both RNA and DNA, including recombinant DNA isolated from a cell and synthetic (e.g., chemically synthesized) DNA. Nucleic acids can be double-stranded or single-stranded. Nucleic acids can be synthesized using oligonucleotide analogs or derivatives (e.g., inosine or phosphorothioate nucleotides). Such oligonucleotides can be used, for example, to prepare nucleic acids with increased resistance to nucleases.

Also included in the invention are genetic constructs (e.g., vectors and plasmids) that include a nucleic acid encoding a mitochondrial-targeted chaperone inhibitor and/or death receptor agonist, autophagy inhibitor, and/or NF-κB signaling pathway inhibitor described herein operably linked to a transcription and/or translation sequence that enables expression, e.g., expression vectors. A selected nucleic acid, e.g., a DNA molecule encoding a peptide described herein, is “operably linked” to another nucleic acid molecule, e.g., a promoter, when it is positioned either adjacent to the other molecule or in the same or other location such that the other molecule can direct transcription and/or translation of the selected nucleic acid.

Also included in the invention are various engineered cells, e.g., transformed host cells, which contain, and optionally express, a nucleic acid disclosed herein. Prokaryotic and eukaryotic cells, e.g., mammalian cells (e.g., tumor cells), yeast, fungi, and bacteria (such as Escherichia coli), and primary and transformed cells, can be host cells. A number of suitable cells are known in the art.

### X. Methods of Screening

Described herein are methods for identifying candidate compounds, e.g., small organic or inorganic molecules (e.g., having a M.W. less than 1,000 Da), oligopeptides, oligonucleotides, carbohydrates, and antibodies that are useful in the methods of treatment described herein. In some methods, a candidate compound is screened for its ability to bind a chaperone, e.g., Hsp90 or TRAP-1. In some methods, a candidate compound is screened for its ability to bind Cyclophilin D. In some methods, a candidate compound is screened for its ability to bind to and/or activate a death receptor. In some methods, candidate compounds are screened in silico by computational methods. Libraries of chemical structures are known in the art.

These candidate compounds can optionally be linked (via covalent or non-covalent interactions) to the mitochondrial-penetrating moieties described herein. In some methods, a candidate compound is screened for its ability to inhibit an interaction between Cyclophilin D and a chaperone, e.g., Hsp90 or TRAP-1. In some methods, a candidate compound is screened for its ability to localize to mitochondria. In some methods, a candidate compound is screened for its ability to induce cell death.

Libraries of Test Compounds

In certain embodiments, screens for candidate compounds that can be used to treat cancer cells use libraries of test compounds. As used herein, a “test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds, e.g., heteroorganic or organometallic compounds.

Test compounds can be screened individually or in parallel. An example of parallel screening is a high throughput drug screen of large libraries of chemicals. Such libraries of candidate compounds can be generated or purchased, e.g., from Chembridge Corp., San Diego, Calif. Libraries can be designed to cover a diverse range of compounds. For example, a library can include 500, 1000, 10,000, 50,000, or 100,000 or more unique compounds. In some cases libraries include classes of compounds with enhanced potential for having anti-cancer activity. Classes of compounds with enhanced potential include known chaperone inhibitors and structurally similar compounds. For example libraries can include ansamycin antibiotics, geldanamycin analogs, and pyrazolopyrimidines and related analogs. A library can be designed and synthesized to cover such a class of chemicals.

The synthesis of combinatorial libraries has been reviewed (see, e.g., Gordon et al., J. Med. Chem. 1994, 37:1385; DeWitt and Czarnik, Acc. Chem. Res. 1996, 29:114; Armstrong et al., Acc. Chem. Res. 1996, 29:123-131; Ellman, J. A., Acc. Chem. Res. 1996, 29:132; Gordon et al., Acc. Chem. Res. 1996, 29:144; Blondelle et al., Trends Anal. Chem. 1995, 14:83; Chen et al., J. Am. Chem. Soc. 1994, 116:2661; U.S. Pat. Nos. 5,359,115; 5,362,899; and 5,288,514; and PCT Publication Nos. WO 92/10092, WO 93/09668, WO 91/07087, WO 93/20242, and WO 94/08051).

Libraries of compounds can be prepared according to a variety of methods, some of which are known in the art. For example, a split-pool strategy can be implemented in the following way: beads of a functionalized polymeric support are placed in a plurality of reaction vessels; a variety of polymeric supports suitable for solid-phase peptide synthesis are known, and some are commercially available (for examples, see, e.g., Bodansky, Principles of Peptide Synthesis, 2nd edition, Springer-Verlag, Berlin (1993)). To each aliquot of beads is added a solution of a different activated amino acid, and the reactions are allowed to proceed to yield a plurality of immobilized amino acids, one in each reaction vessel. The aliquots of derivatized beads are then washed, pooled (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a biased library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The split-pool strategy can result in a library of peptides, e.g., modulators, which can be used to prepare a library of test compounds for use in the screens described herein. In another illustrative synthesis, a diversomer library is created by the method of DeWitt et al., Proc. Natl. Acad. Sci. USA 1993, 90:6909. Other synthesis methods, including the “tea-bag” technique, described in Houghten et al., Nature 1991, 354:84, can also be used to synthesize libraries of compounds according to the subject invention.

Libraries of compounds can be screened to determine whether any members of the library have chaperone, e.g., Hsp90 or TRAP-1, inhibitory activity, and, if so, to identify the inhibitor. Additionally, libraries of compounds can be screened to determine whether any members of the library have death receptor agonistic activity, and, if so, to identify the agonist. Methods of screening combinatorial libraries have been described. See, e.g., Gordon et al., J. Med. Chem., supra. Soluble compound libraries can be screened to isolate death receptor agonists and/or inhibitors of chaperones, e.g., Hsp90 or TRAP-1, followed by identification of the isolated ligands by conventional techniques (e.g., mass spectrometry, NMR, and the like). Screens are described herein.

Screens

In certain embodiments, screening for compounds capable of inhibiting chaperones in mitochondria can include identifying from a group of test compounds those that (i) inhibit and/or bind to a molecular chaperone, (ii) inhibit interaction between a molecular chaperone and Cyclophilin D, and/or (iii) decrease levels of chaperones in tumor cell mitochondria. Test compounds that exhibit one or more of activities (i), (ii), or (iii) are referred to herein as “candidate compounds.” Screening assays can optionally include further testing candidate compounds for their ability to modulate proliferation of cancer cells in vitro or in vivo. Screening assays of the present invention may be carried out in whole cell preparations and/or in ex vivo cell-free systems. In some embodiments, test compounds or candidate compounds are linked to a mitochondrial-penetrating moiety.

In certain embodiments, screening for compounds capable of agonizing death receptors can include identifying from a group of test compounds those that (i) activate and/or bind to a death receptor, and/or (ii) increase levels of a death receptor in tumor cells. Test compounds that exhibit one or more of activities (i) and (ii) are referred to herein as “candidate compounds.” Screening assays can optionally include further testing candidate compounds for their ability to modulate proliferation of cancer cells in vitro or in vivo. Screening assays of the present invention may be carried out in whole cell preparations and/or in ex vivo cell-free systems.

Binding of a test compound to a cell-free sample that includes a chaperone protein can be detected, for example, in vitro by reversibly or irreversibly immobilizing the chaperone protein on a substrate, e.g., the surface of a well of a plate (e.g., 96-well polystyrene microtitre plate). For example, microtitre plates can be coated with the chaperone protein, or a fragment thereof, washed and blocked (e.g., with BSA) to prevent non-specific binding of test compounds to the plates. The chaperone protein is then cross-linked to the plate. Test compounds are added to the coated plate under a number of conditions (e.g., at 37° C. for 0.5-12 hours). The plate can then be rinsed and binding of the test compound to the chaperone protein can be detected by any of a variety of art-known methods. For example, an antibody that specifically binds to the chaperone protein can be used in an immunoassay. If desired, the antibody can be labeled (e.g., fluorescently or with a radioisotope) and detected directly (see, e.g., West and McMahon, J. Cell Biol. 1977, 74:264). Alternatively, a second antibody can be used for detection (e.g., a labeled antibody that binds to the anti-chaperone protein antibody). Test compounds that bind to the chaperone protein can be detected by their ability to inhibit binding of antibody to immobilized chaperone protein. In an alternative detection method, the test compound is labeled (e.g., with a radioisotope, fluorophore, chromophore, or the like), and the binding of a test compound to the chaperone protein is detected by detecting label that is immobilized on the substrate.

In still another embodiment, test compounds are immobilized on a substrate, e.g., to a microtitre plate as described above, incubated with a cell free sample that includes a chaperone protein (or a fragment thereof), washed, and the ability of the chaperone protein to bind to an immobilized test compound is detected. For example, Hsp90 or the extracellular domain of a death receptor (or a fragment thereof) can be produced as a fusion protein with a protein that can be detected optically, e.g., green fluorescent protein or a variant thereof (which can be detected under UV light), and the ability of the fusion protein to bind the test compound is detected. Alternatively, a chaperone or death receptor fragment can be produced as a fusion protein with an enzyme having a detectable enzymatic activity, such as horseradish peroxidase, alkaline phosphatase, β-galactosidase, or glucose oxidase. Genes encoding all of these enzymes have been cloned and are available for use by skilled practitioners. If desired, the fusion protein can include an antigen, which can be detected and measured with a polyclonal or monoclonal antibody using conventional methods. Suitable antigens include enzymes (e.g., horse radish peroxidase, alkaline phosphatase, and β-galactosidase) and non-enzymatic polypeptides (e.g., serum proteins, such as BSA and globulins, and milk proteins, such as caseins). In these methods, the ability of the chaperone fusion protein to bind to a test compound is detected.

To identify polypeptides that bind to a chaperone protein a two-hybrid assays of protein/protein interactions can be used (see, e.g., Chien et al., Proc. Natl. Acad. Sci. U.S.A. 1991, 88:9578; Fields et al., U.S. Pat. No. 5,283,173; Fields and Song, Nature 1989, 340:245; Le Douarin et al., Nucl. Acids Res. 1995, 23:876; Vidal et al., Proc. Natl. Acad. Sci. U.S.A. 1996, 93:10315-20; and White, Proc. Natl. Acad. Sci. U.S.A. 1996, 93:10001-03). Kits for practicing various two-hybrid methods are commercially available (e.g., from Clontech; Palo Alto, Calif.).

In certain other embodiments, the interaction of a chaperone protein or death receptor, or fragment thereof, and test compound is detected by fluorescence resonance energy transfer (FRET) between a donor fluorophore covalently linked to either the chaperone protein or the test compound and an acceptor fluorophore covalently linked to either the chaperone protein or the test compound, wherein the acceptor and donor fluorophore are not both linked to the chaperone protein or the test compound, and there is suitable overlap of the donor emission spectrum and the acceptor excitation spectrum to give efficient nonradiative energy transfer when the fluorophores are brought into close proximity through the chaperone protein-test compound interaction.

In some methods, test compounds that are candidate compounds for the treatment of tumors or cancer can be identified by contacting a test compound to a sample that includes one or more chaperone proteins and CypD, and then screening for decreased interaction between a chaperone and CypD. In one embodiment, a cell-free system is used to determine if recombinant TRAP-1 or recombinant Hsp90 co-immunoprecipitate with recombinant CypD in the presence of a test compound.

In some methods, test compounds that are candidate compounds for the treatment of tumors or cancer are contacted with one or more tumor cells and are evaluated for decreased expression of the chaperone. In a related method, one or more test compound is contacted to a tumor cell that expresses a recombinant chaperone, and the cells are evaluated for decreased expression of the recombinant chaperone. Expression of a chaperone can be measured, for example, by Northern blot, RT-PCR analysis, RNAse protection analyses, Western blot, enzyme-linked immunosorbent assay (ELISA), radioimmunoassay (RIA) and fluorescent activated cell sorting (FACS). The level of expression in the presence of the test molecule, compared with the level of expression in its absence, will indicate whether or not the test compound inhibits the expression of the chaperone. In one embodiment, the test compound is a small interfering RNA (siRNA).

Having identified a test compound as a candidate compound, the candidate compound can be further tested, e.g., in proliferation assays of tumor cells using in vitro or in vivo model systems. In vitro proliferation assays include contacting a candidate compound to a culture of tumor cells, e.g., Raji cells, and evaluating the ability of the candidate compound to induce apoptosis in and/or prevent proliferation of the cultured cells. In vivo tumor assays include administering a candidate compound to an animal model, e.g., a rodent, with a tumor or a predisposition to develop a tumor, and subsequently evaluating the candidate compound's ability to inhibit tumor development or tumor proliferation in the animal. Exemplary animal models of cancer include animals with xenografted cancer cells. Other animal models include rodents with a genetic predisposition to develop tumors, e.g., mice bearing mutant forms of (i) adenomatous polyposis coli (APC) gene (e.g., a multiple intestinal neoplasia (APC^{Min}) mouse (see, e.g., Haigis et al., Proc. Natl. Acad. Sci. U.S.A. 101:9769-9773, (2004)), (ii) mut-s homologue-2 (Msh2) gene (see, e.g., Kohonen-Corish et al., Cancer Res. 62:2092-2097, (2002)), and/or (iii) MutL homologue-1 (Mlh1) gene (see, e.g., Cohen et al., Cell 85:1125-1134, (1996)). The C57BL/6J-Apc^{Min} mouse is available from Jackson Harbor Labs (Bar Harbor, Me.). Alternatively, an animal model can be exposed to carcinogenic chemicals such as dimethylhydrazine derivatives or heterocyclic amines, such as 2-amino-1-methyl-6-phenylimidazo[4,5-b]pyridine (PhIP), that have been reported to induce tumors in animal models.

In some methods, candidate compounds for the treatment of tumors or cancer can be further tested for apoptosis-inducing activity by contacting the candidate compound to a sample that includes one or more tumor cells, and then screening for decreased cell viability. In one embodiment, decreased cell viability is measured using an MTT (3-(4,5-dimethylthiazol-2-yl)-2,5-diphenyltetrazolium bromide) reduction assay. The colorimetric MTT assay, developed by Mossman (J. Immunol. Meth. 1983, 65:55-63), is based on the conversion of the water-soluble MTT to an insoluble purple formazan. The formazan is then solubilized, and its concentration determined by absorbance at 570 nm. The methods can be performed, e.g., as described in Plescia et al. (Cancer Cell 2005 7:457-68). Other viability assays can also be used.

In some methods, candidate compounds for the treatment of tumors or cancer can be further tested by contacting a test compound to a sample that includes one or more tumor cells, and then screening for increased apoptosis. In one embodiment, increased apoptosis is evident as increased caspase activity as determined by DEVDase hydrolysis. Methods for measuring apoptosis are well known in the art.

In some methods, candidate compounds for the treatment of tumors or cancer can be further tested for their ability to disrupt mitochondrial membrane integrity. For example, candidate compounds can be further tested for their ability to induce a change in mitochondrial membrane potential, increase cytochrome c release, or increase Smad release. In one embodiment, cells are treated with a candidate compound and further treated with a mitochondrial membrane potential-sensitive fluorescent dye JC-1, and analyzed for changes in green/red fluorescence ratio by flow cytometry.

In some methods, candidate compounds for the treatment of tumors or cancer can be further tested for their ability to inhibit chaperone activity. For example, candidate compounds can be further tested for their ability to induce degradation of Akt, an Hsp90 client protein.

Medicinal Chemistry

Once a compound (or agent) of interest has been identified, standard principles of medicinal chemistry can be used to produce derivatives of the compound. Derivatives can be screened for improved pharmacological properties, for example, efficacy, pharmaco-kinetics, stability, solubility, and clearance. The moieties responsible for a compound's activity in the assays described above can be delineated by examination of structure-activity relationships (SAR) as is commonly practiced in the art. A person of ordinary skill in pharmaceutical chemistry can modify moieties on a candidate compound or agent (i.e., a lead compound) and measure the effects of the modification on the efficacy of the compound or agent to thereby produce derivatives with increased potency. For an example, see Nagarajan et al., J. Antibiot. 41:1430-1438, (1988). Furthermore, if the biochemical target of the compound (or agent) is known or determined, the structure of the target and the compound can inform the design and optimization of derivatives. Molecular modeling software is commercially available (e.g., from Molecular Simulations, Inc.) for this purpose.

### EXAMPLES

The invention is further described in the following examples, which do not limit the scope of the invention described in the claims.

### Example 1

### Gamitrinibs Sensitize Tumor Cells to TRAIL-Mediated Apoptosis

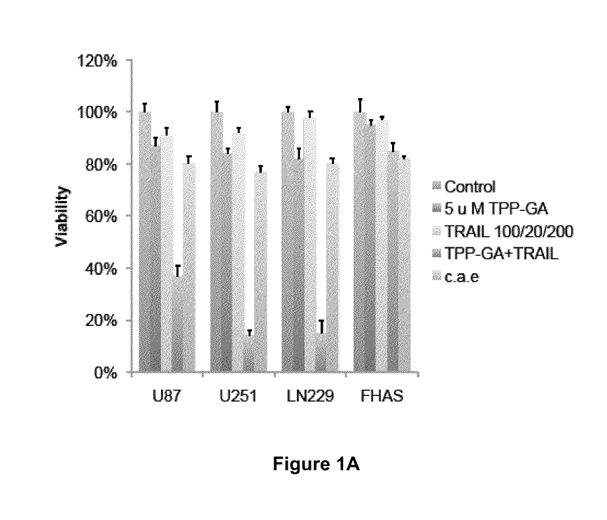

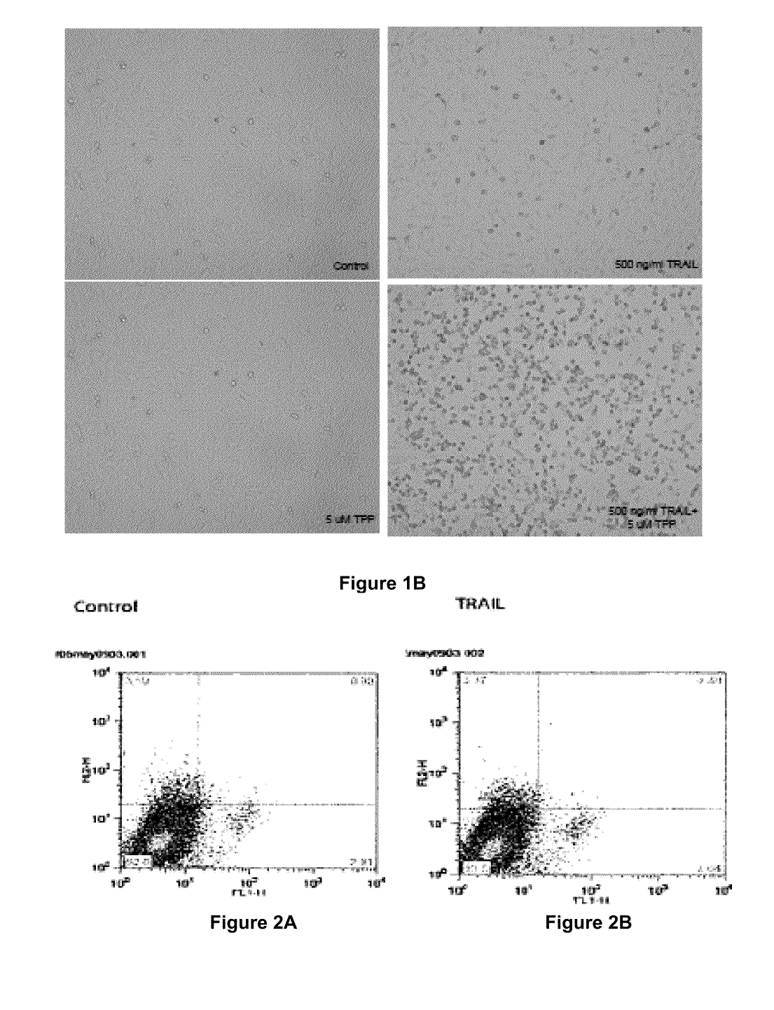

The effect of gamitrinibs and death receptor agonists on tumor cells was assayed in vitro using tumor cell lines. Glioma cell lines U87, U251, and LN229 all contain a mutation in the tumor suppressor p53 and are resistant to TRAIL treatment. The effect of triphenylphosphonium geldanamycin (TPP-GA) and TRAIL, singly and in combination, on U251 and LN229 cellular viability was evaluated by 3-(4,5-dimethylthiazol-2-yl)-2,5-diphenyltetrazolium bromide (MTT) assay (Kang et al., 2009, J. Clin. Invest. 119:454-464). Treatment with 15 ng/ml or 500 ng/ml of TRAIL for 16 hours reduced cellular viability to 80% in U251 and 98% in LN229 glioma cells, respectively (FIG. 1A). Treatment with 5 μM of TPP-GA reduced cellular viability to 78% in both LN229 and U251 glioblastoma cells (FIG. 1A). Combined treatment with 5 μM of TPP-GA and TRAIL reduced cellular viability in U251 to 15%±6%, p<0.05 and in LN229 to 12%±5%, p<0.05 (FIG. 1). The calculated additional effect (c.a.e.) for the single treatments was 63% for U251 and 77% for LN229. The loss of viability was confirmed by cell counting (see FIG. 1B for example with LN229 cells). Similar results were obtained in U87 cells, a breast cancer cell line (MCF-7), and a prostate cancer cell line (PC-3). Fetal human astrocytes (FHAS) were not affected by the combination treatment consisting of TRAIL and Gamitrinibs, suggesting that the reduction in viability was specific to cancer cells (FIG. 1A).

The pan-caspase inhibitor benzyloxycarbonyl-Val-Ala-Asp(OMe)-fluoromethyl-ketone (zVAD-fmk) significantly attenuated TRAIL+Gamitrinib mediated apoptosis, suggesting that Gamitrinibs sensitize cancer cells to TRAIL-mediated cell death by enhanced activation of caspases.

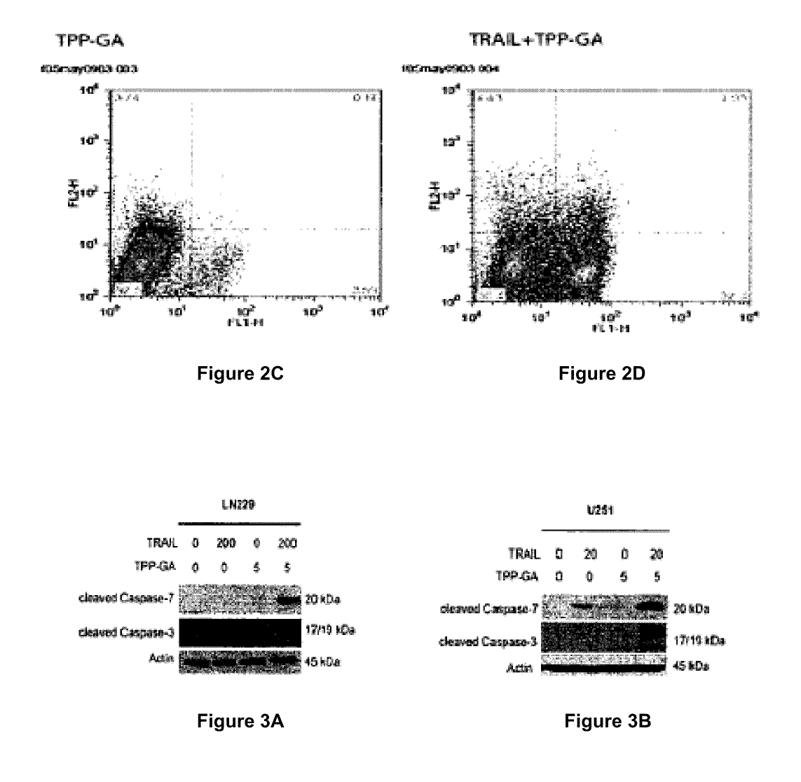

To demonstrate that the reduction in viability was due to apoptosis, treated cells were stained with Annexin V and propidium iodide (PI) and assayed by flow cytometry (FIGS. 2A-D). Single treatment with either 5 μM of TPP-GA (FIG. 2C) or 25 ng/ml of TRAIL (FIG. 2B) exhibited no significant effects in U251 cells, whereas the combination of both agents resulted in a significant increase in Annexin V positive and PI-negative tumor cells (FIG. 2D), indicating that the combination induced apoptosis in the cells. This example demonstrates that mitochondrial-targeted chaperone inhibitors and death receptor agonists act synergistically to induce apoptosis of cancer cells.

### Example 2

### Gamitrinibs Facilitate TRAIL-Mediated Apoptosis in Glioma Cells by Activation of Initiator and Effector-Caspases

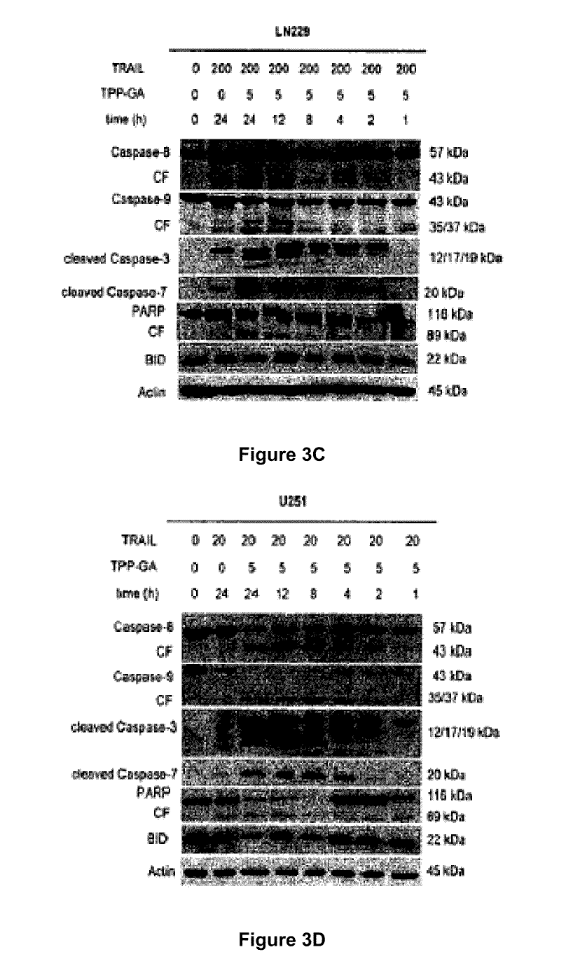

Western blotting was employed to elucidate the proteolytic mechanism in TRAIL and TRAIL+Gamitrinib-induced apoptosis. Since Gamitrinibs augment TRAIL-induced apoptosis, the activation/cleavage of caspases-3, -7, -8, -9, BH3 interacting domain death agonist (BID), and Poly-ADP-ribose polymerase (PARP) was examined. Treatment for 16 hours of LN229 (FIG. 3A) and U251 (FIG. 3B) cells with either 5 μM TPP-GA or 200 or 20 ng/ml TRAIL, respectively, did not or only weakly induce cleavage of caspase-3 or -7. Combining TRAIL with TPP-GA led to a significant increase in cleaved fragment of caspases-3 and -7. The time course of activation/cleavage of caspases-3, -7, -8, -9, BID, and PARP over 24 hours was also examined following treatment of LN229 (FIG. 3C) or U251 (FIG. 3D) cells with 5 μM TPP-GA and 200 or 20 ng/ml TRAIL, respectively. The combination of TRAIL and TPP-GA led to a significant increase in cleaved fragment of PARP and activated cleaved caspases-7, -8, and -9 in both U251 and L229. Treatment with zVAD-fmk abolished cleavage of caspases induced by the combination of TRAIL and TPP-GA. This example demonstrates that mitochondrial-targeted chaperone inhibitors efficiently augmented death receptor agonist-mediated apoptosis by both the intrinsic and extrinsic apoptotic pathways.

### Example 3

### Gamitrinibs Sensitize In Vivo Xenografts to TRAIL-Mediated Tumor Regression

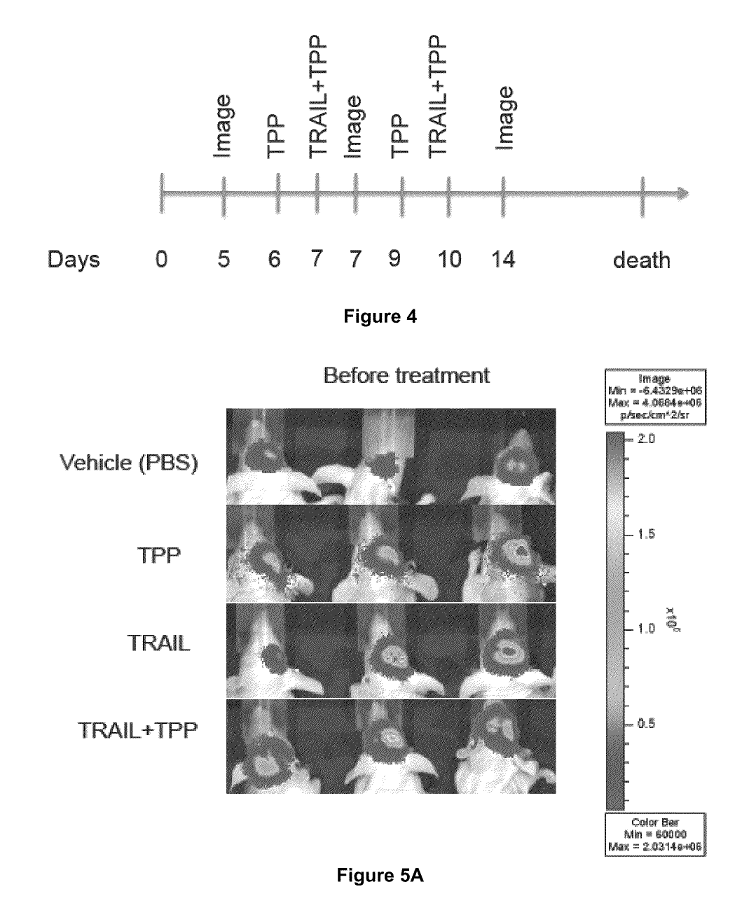

Since Gamitrinib-TPP dramatically sensitizes cancer cells in vitro it was of highest interest to assess efficacy in vivo. To this end, efficacy of TRAIL, TPP, or the combination of both was determined in an orthotopic glioblastoma tumor model. Twenty nude mice were intracranially injected with U87-luc glioblastoma cells. U87 glioblastoma cells stably transfected with a luciferase expression plasmid (U87-Luc) were suspended in sterile PBS, pH 7.2, and stereotactically implanted (1×10⁵) in the right cerebral striatum of immunocompromised CB17 SCID/beige female mice (Charles River Laboratories). Animals with established tumors were randomized in four groups (5 animals per group) and treated as indicated in FIG. 4. Treatment group 1 received only vehicle solutions. Group 2 received TRAIL intracranially (2 μg) and carrier solution intraperitoneally. Group 3 received PBS intracranially and TPP intraperitoneally in a carrier solution consisting of 20% Cremophor in PBS. Group 4 received both TRAIL and TPP. Tumor growth was assessed after each intracranial injection by application of 58 mg/kg D-luciferin and subsequent bioluminescence imaging using a Xenogen In Vivo Imaging System. All experiments involving animals were approved by an Institutional Animal Care and Use Committee.

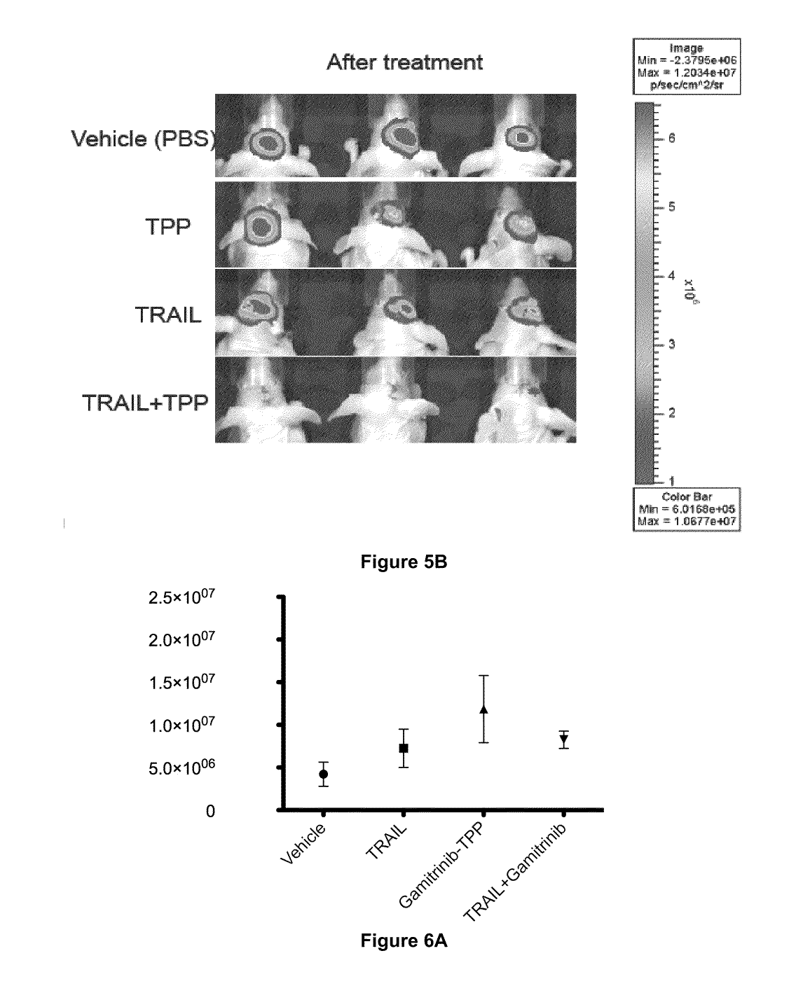

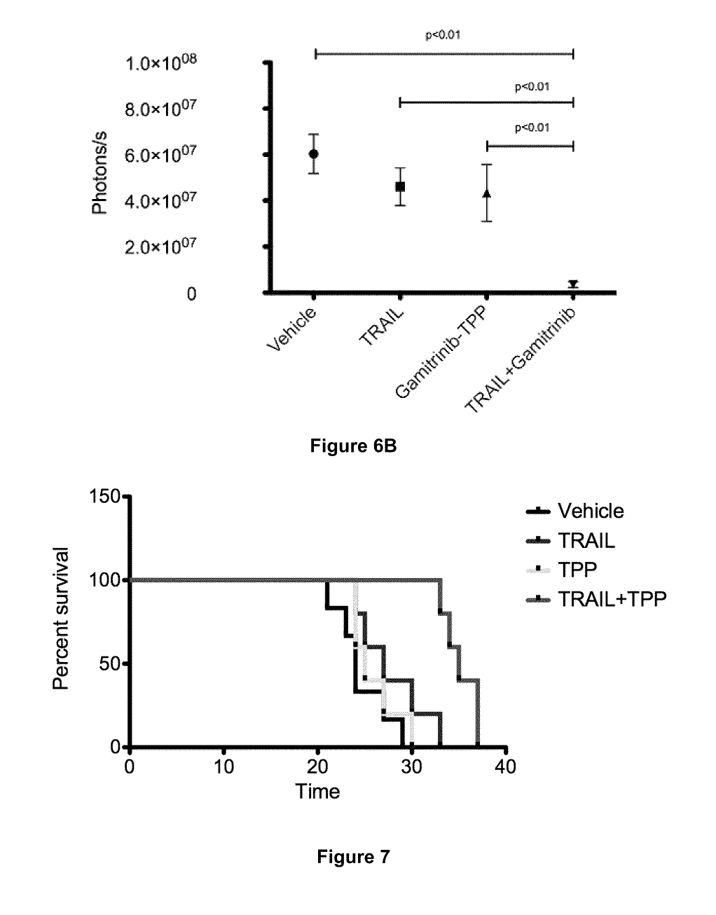

Five days after injection of U87-luc glioblastoma cells, the mice were scanned, quantified and according to signal strength randomized into 4 treatment groups (Group 1: Vehicle, Group 2: TRAIL, Group 3: TPP, Group 4: TRAIL+TPP) (FIGS. 5A, 6A). Following treatment, animals receiving just single treatment of either TRAIL (intracranially) or TPP (intraperitoneally) did not show any significant reduction in tumor size (FIGS. 5B, 6B). However, the combination of TRAIL and TPP resulted in a significant tumor regression (TRAIL+TPP vs. TRAIL, p<0.01; TRAIL+TPP vs. vehicle, p<0.01) (FIG. 5B, 6B). It is noteworthy that this tumor regression was mediated without any significant weight loss among the different groups.

In agreement with the bioluminescence data, survival was significantly prolonged in TRAIL+TPP animal group compared to single treatment groups (TRAIL+TPP vs. TRAIL, p<0.01; TRAIL+TPP vs. TPP, p<0.01; TRAIL+TPP vs. vehicle, p<0.01) (FIG. 7).

This example demonstrates that the combination of a mitochondrial-targeted chaperone inhibitor (gamitrinib-TPP) and a death receptor agonist (TRAIL) provided a synergistic antitumor effect as compared to each treatment individually.

### Example 4

### Induction of Cell Death by Combination of Gamitrinib and an Autophagy Inhibitor and Affect of Gamitrinib on NF-κB Signaling Pathway

Experiments were performed to further determine the whether the combination of gamitrinib and an autophagy inhibitor would have an effect on cancer cell death. Additional experiments were performed to determine the effect of gamitrinib treatment on NF-κB signaling pathway activity in cancer cells.

### Cells and Cell Cultures

Human glioblastoma cell lines LN229 (p53 mutant; PTEN, WT), U87 (p53 WT; PTEN mutant), U251 (p53 mutant), prostate adenocarcinoma PC3, breast adenocarcinoma MCF-7, and human epithelial kidney (HEK) 293T were purchased from ATCC. Cells were cultured in DMEM Glutamax-I medium (Invitrogen) containing 10% FBS and 1% penicillin/streptomycin (Invitrogen) at 37° C. in a 10% CO₂ humidified atmosphere. Patient-derived cultures of glioblastoma cells were established from fresh, surgically resected samples of WHO grade IV and grade III glioblastomas (GS620, GS48, and AS515) obtained from the Division of Neurosurgery, University of Massachusetts Medical School. Tissue samples were minced in small pieces and transferred to plastic tissue culture flasks (Falcon; BD); culture outgrowths were established in DMEM medium containing 10% FCS and 1% penicillin/streptomycin at 37° C. in 10% CO₂. Upon reaching confluence, the cell monolayers were harvested by a brief incubation with trypsin/EDTA (Viralex; PAA Laboratories) and confirmed as mycoplasma free by staining with 4,6-diamidino-2 phenylindole (Roche Diagnostics). The glial origin of these cultures was confirmed by staining for α-glial fibrillary acidic protein. In contrast, antibodies against endothelial cell markers, CD31 (Pharmingen), factor VIII (Dako), or neuronal neurofilament proteins 70, 160, and 200 (all from Progen), were unreactive. Primary FHAS were obtained from ScienCell Research Laboratories and maintained in culture as described above or, alternatively, transformed by expression of the SV40 oncogene.

### Antibodies

Antibodies to LC3 (Cell Signaling), COX-IV (1:1000; Calbiochem), Hsp70 (1:1000; Abcam), FLIP-L, long form (1:250; CST) FLIP-S, short form (1:250; CST), β-actin (1:2000; Sigma-Aldrich), C/EBP/β (1:1000; CST), and CHOP (1:1000; CST) were used.

### Chemicals

The complete chemical synthesis, HPLC profile, and mass spectrometry of mitochondria-targeted small molecule Hsp90 antagonists Gamitrinibs has been reported previously (Kang et al., J. Clin. Invest. 119:454-464, 2009). GamitrinibTPP (G-TPP) was used in this study. Nonmitochondrial-permeable Hsp90 inhibitor 17-allylamino demethoxygeldanamycin (17-AAG) was obtained from LC-Laboratories. Recombinant TRAIL was obtained from Peprotech Inhibitors of phagosome formation bafilomycin A (BF) or 3-methyladenine (3-MA) were obtained from Sigma-Aldrich.

### Analysis of Cell Viability and Autophagy

The various cell types were seeded in triplicate onto 96-well plates at 2×10³ cells/well, treated with vehicle, G-TPP, or nontargeted 17-AAG (0-20 μM) for up to 24 hours, and quantified for metabolic activity by a 3(4,5-dimethyl-thyazoyl-2-yl)2,5 diphenyltetrazolium bromide (MTT) colorimetric assay with absorbance at 405 nm, as described in Plescia et al. (Cancer Cell 7:457-468, 2005). Changes in protein expression under the various combination regimens were determined by immunoblotting.

For autophagy, glioblastoma cells were transfected with an LC3-GFP cDNA by FuGENE HD (Roche), treated with vehicle, nontargeted 17-AAG, or G-TPP, and analyzed by fluorescence microscopy. A cell was scored as autophagic when exhibiting a punctate GFP labeling with more than 10 LC3-GFP dots/cell. An average of 200 cells was counted in 4-6 independent fields per condition. In some experiments, G-TPP-treated cells were incubated with pharmacologic inhibitors of autophagy, BF, or 3-MA, and analyzed after 16 hours for modulation of cell viability by MTT.

### Electron Microscopy

Subconfluent monolayers of LN229 cells were incubated with PBS, pH 7.2, or G-TPP or nontargeted 17-AAG (5-10 μM) for 16 hours at 37° C. Cells were washed briefly in 0.1 M PBS, pH 7.4, fixed in buffered 2.5% glutaraldehyde (GA) with or without 4% formaldehyde (FA) or for 30 minutes at 22° C., post-fixed in 1% osmium tetroxide, and processed by standard methods for epoxy embedding. In some experiments, cells were fixed in buffered 3% FA with 0.1% GA for 25 minutes at 22° C., washed, quenched with NH₄Cl, and dehydrated for embedding in LR White resin. Ultrathin sections on nickel grids were treated for heat-induced antigen retrieval and blocked with buffered 1% BSA, as described in published protocols (Yamashita et al., Methods Mol. Biol. 657:237-248, 2010). Samples were incubated with an antibody to COX IV, followed by a colloidal gold-conjugated (12 nm) secondary antibody of appropriate specificity (Jackson ImmunoResearch). Grids were briefly post-fixed with 2% GA, washed, air dried, and then exposed to osmium tetroxide vapors for 20 minutes. Ultrathin sections were stained with uranyl acetate and lead citrate before examination in a Philips CM10 transmission electron microscope.

### Promoter Activity

Tumor cell types were transfected with NF-κB (Mehrota et al., Cancer Cell 17:53-64, 2010), p53, or FLIP luciferase promoter construct (pGL3-FLIP1500; Addgene #16016) using FuGENE HD. After 24 hours at 37° C., cells were stimulated with G-TPP or 17-AAG (0-10 μM) for 6 hours, harvested, and analyzed for β-galactosidase-normalized luciferase activity in a luminometer, as described (Mehrota et al., Cancer Cell 17:53-64, 2010). In some experiments, β-galactosidase-normalized NF-κB promoter activity was examined after cell stimulation with TNF-α, as described (Mehrota et al., Cancer Cell 17:53-64, 2010).

### Transfections

Glioblastoma cell types were transfected with nontargeting or CHOP-directed SMARTpoo1 siRNA (Dharmacon cat. no. L-004819-00-0005) using Oligofectamine 2000, characterized for protein knockdown by immunoblotting, and processed for further studies. In some experiments, glioblastoma cell types were transfected with control pcDNA3 or plasmid encoding WT CHOP, a CHOP deletion mutant lacking the leucine zipper motif (CHOP-ALeu) (Du et al., J. Immunol. 182:7201-7211, 2009), or C/EBPβ, by lipofectamine. Cells were examined for recombinant protein expression by immunoblotting and analyzed for changes in NF-κB promoter activity with or without TNF-α.

In some experiments, LN229 cells were transfected with SMART pool siRNA (Thermo Scientific Dharmacon, Lafayette, Colo.) and left untreated or treated with G-TPP. The transfected LN229 cells were examined for protein expression by immunoblotting or analyzed at 16 hours for cell viability using MTT.

### Statistics

Data were analyzed by 2-sided unpaired t tests using a GraphPad software package (Prism 4.0) for Windows. For experiments of luciferase promoter expression or MTT, data are expressed as mean±SD or mean±SEM of replicates of a representative experiment out of at least 2 independent determinations. P≦0.05 was considered statistically significant.

### Results

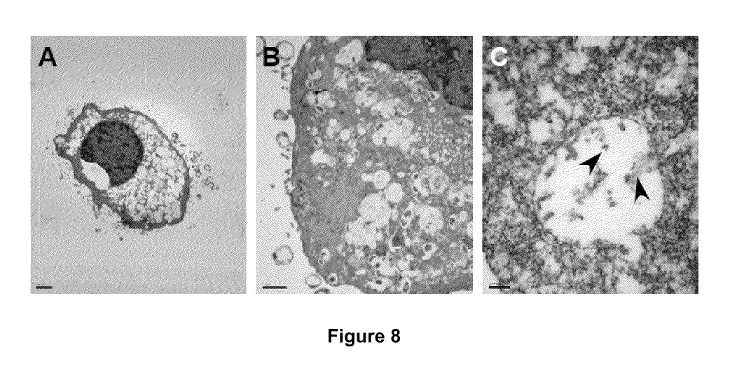

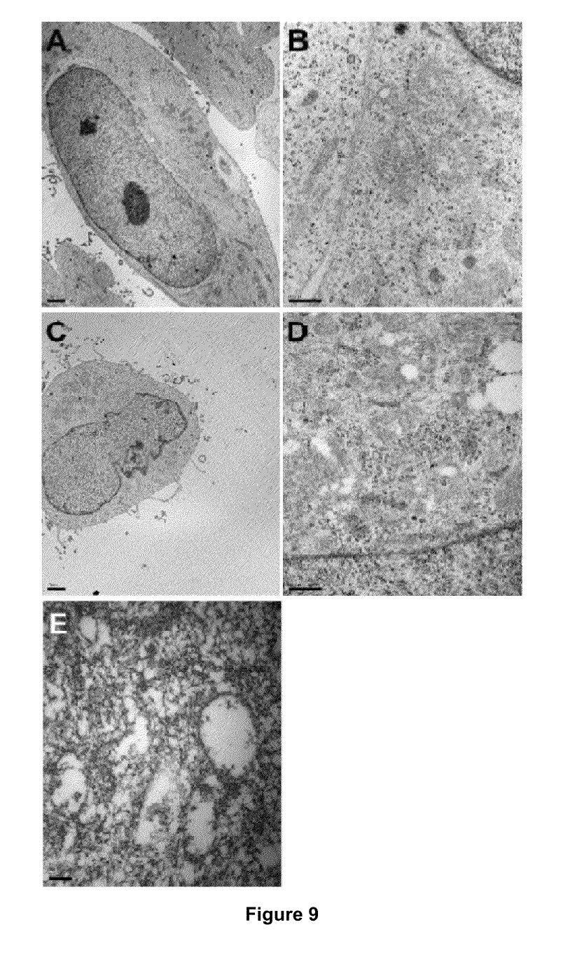

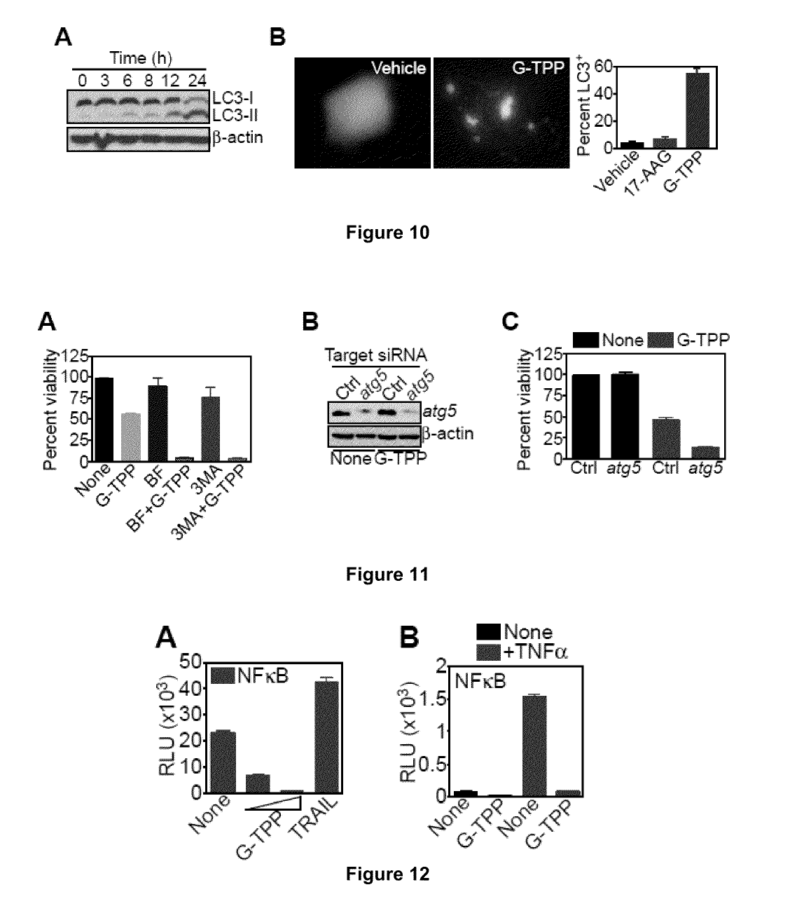

Glioblastoma cells treated with sub-optimal concentrations of gamitrinib-TPP of 5-10 μM became completely filled with cytoplasmic vacuoles (FIG. 8A). These structures contained subcellular material (FIG. 8B) that comprised mitochondria, as determined by immuno-gold labeling and electron microscopy with an antibody to COX-IV (FIG. 8C). In contrast, tumor cells treated with vehicle (FIGS. 9A and 9B), or 17-AAG (FIGS. 9C and 9D) were unremarkable, with fine chromatin structure and normal organelles, and a non-binding IgG was unreactive (FIG. 9E). Over time, G-TPP-treated cells also accumulated a lapidated form of the ubiquitin-like protein, light chain 3 (LC3) (FIG. 10A) and exhibited a punctate fluorescence pattern when transfected with an LC3 plasmid fused to Green Fluorescence Protein (GFP) (FIG. 10B). Collectively, these are all hallmarks of autophagy, a compensatory cellular response that attempts to preserve cell viability during stress by self-digesting damaged organelles. Accordingly, pharmacologic inhibitors of phagosome formation, bafilomycin A or 3-methyladenine (FIG. 11A) or small interfering RNA (siRNA) knockdown of the essential autophagy gene, atg5 (FIG. 11B), enhanced glioblastoma cell death induced by suboptimal concentrations of G-TPP (FIG. 11C).

The data show that autophagy is activated in glioblastoma cells by sub-optimal (e.g., non-cytotoxic) concentrations of gamnitrinib-TPP. These data suggest that the combination of a mitochondrial-targeted chaperone inhibitor and an autophagy inhibitor can provide enhanced anti-cancer activity (e.g., cancer cell apoptosis) compared to the administration of a mitochondrial-targeted chaperone inhibitor and an autophagy inhibitor administered alone or the sum of the anti-cancer activity (e.g., cancer cell apoptosis) observed for the administration of a mitochondrial-targeted chaperone inhibitor and an autophagy inhibitor alone, and further suggest that the co-administration of a mitochondrial-targeted chaperone inhibitor and an autophagy inhibitor has little or reduced toxicity for normal organs or tissues.

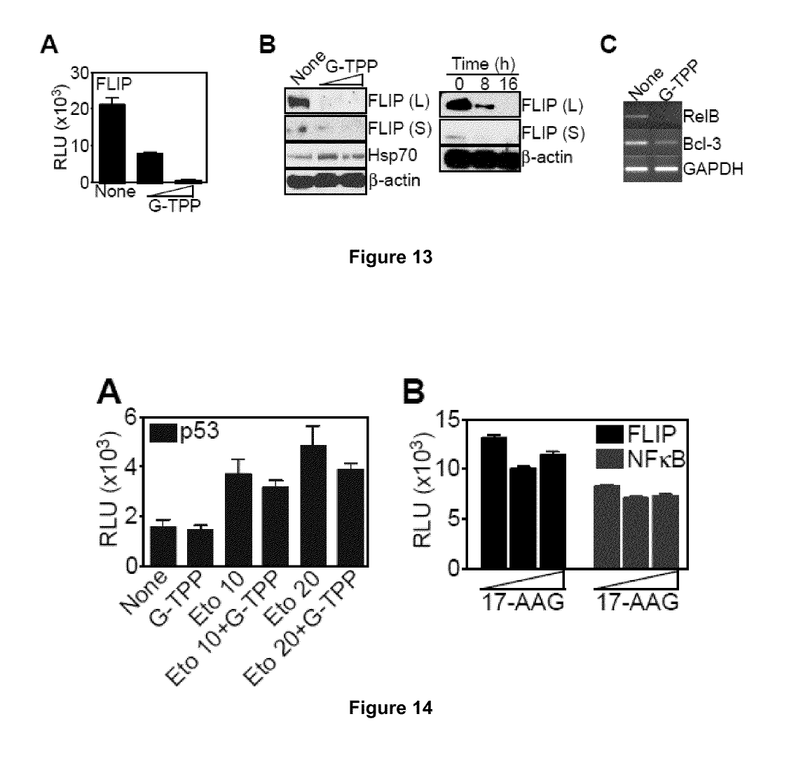

G-TPP treatment of glioblastoma cells resulted in complete ablation of NF-κB-mediated gene expression, either constitutively (FIG. 12A) or after stimulation with TNF-α (FIG. 12B). This was associated with a rapid downregulation of multiple NF-κB target genes, including FLIP promoter activity (FIG. 13A) and protein expression (FIG. 13B), as well as RelB and bcl-3 mRNA (FIG. 13C). Gamitrinib-TTP did not exert a generic suppressive effect on gene expression, as etoposide-mediated induction of p53 luciferase promoter activity was unaffected by G-TPP (FIG. 14A). As control, non-mitochondrially-targetted Hsp90 inhibitor 17-AAG had no effect on NF-κB or FLIP promoter activity (FIG. 14B).

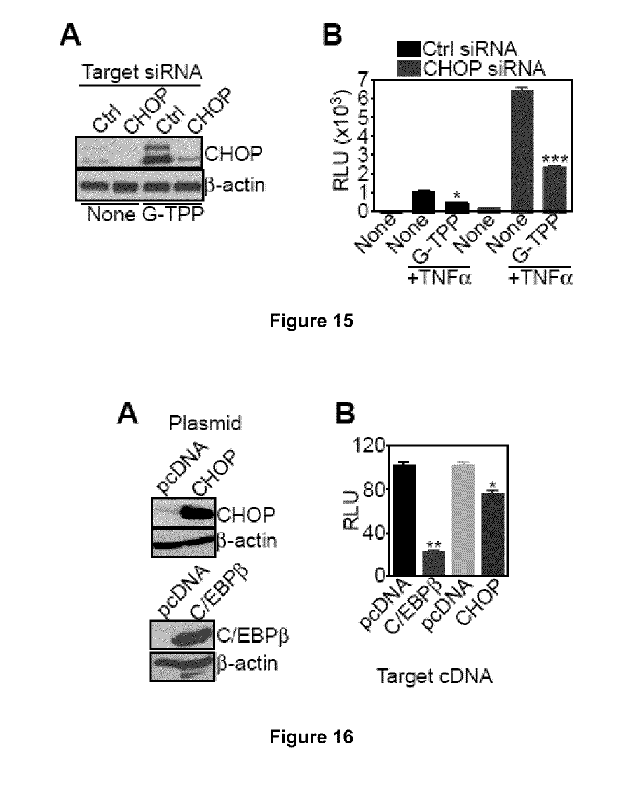

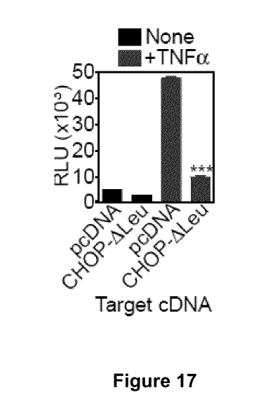

Additional experiments were performed to determine whether the transcription effectors of the mitochondrial unfolded protein response (UPR), CHOP and/or C/EBPβ mediated the inhibition of NF-κB activity under these conditions. SiRNA silencing of CHOP (FIG. 15A) produced constitutively higher NF-κB activity in glioblastoma cells, as compared with control transfectants (FIG. 15B), and partially restored NF-κB-dependent transcription in the presence of G-TPP (FIG. 15B). Reciprically, glioblastoma cells transfected with CHOP or C/EBPβ (FIG. 16A) exhibited reduced NF-κB activity in response to TNFα, whereas a control plasmid had no effect (FIG. 16B). To test whether CHOP DNA binding was required for inhibition of NF-κB, glioblastoma cells were transfected with a CHOP mutant that lacks the leucine zipper domain (ALeu). Expression of this CHOP mutant, but not control pcDNA, suppressed NF-κB activity in response to TNF-α indistinguishably of wild type CHOP (FIG. 17).

These data suggest that by decreasing NF-κB activity, low concentrations of a mitochondrial-targeted chaperone inhibitor may overcome drug resistance in tumor cells, and restore their sensitivity to any anti-cancer therapeutic that is antagonized by the pro-survival function of NF-κB signaling. As shown in the data herein, G-TPP was able to restore sensitivity to glioblastoma cells to TRAIL-induced apoptosis: a cell type that is completely resistant to TRAIL treatment alone. These data further suggest that the combination a mitochondrial-targeted chaperone inhibitor with an NF-κB signaling pathway inhibitor can increase or induce cancer cell death (e.g., apoptosis), and may result in an increase in cancer cell death that is greater than the amount of cancer cell death achieved when a mitochondrial-targeted chaperone inhibitor or a NF-κB signaling pathway inhibitor are administered alone, or results in a degree of cancer cell death that is greater than the sum of the amount of cancer cell death achieved by a mitochondrial-targeted chaperone inhibitor and a NF-κB signaling pathway inhibitor when administered alone. These data also indicate that cancer cells in a subject with up-regulated NF-κB signaling activity following treatment with a cancer therapeutic, can be killed by administering a combination of a mitochondrial-targeted chaperone inhibitor and the cancer therapeutic that up-regulates NF-κB signaling activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of enhancing apoptosis in one or more mammalian cells, comprising contacting the one or more cells with at least one mitochondrial-targeted chaperone inhibitor and at least one agent selected from the group consisting of: a death receptor agonist, an autophagy inhibitor, and a NF-κB signaling pathway inhibitor, wherein the at least one mitochondrial-targeted chaperone inhibitor and the at least one agent are in an amount sufficient to enhance apoptosis in the one or more cells.

2. A method of treating of a proliferative disorder in a subject comprising administering to said subject at least one mitochondrial-targeted chaperone inhibitor and at least one agent selected from the group consisting of: a death receptor agonist, an autophagy inhibitor, and a NF-κB signaling pathway inhibitor, wherein the at least one mitochondrial-targeted chaperone inhibitor and the at least one agent are administered in an amount sufficient to treat of a proliferative disorder in the subject.

3. A method for enhancing cancer or tumor cell death, the method comprising administering to the subject at least one mitochondrial-targeted chaperone inhibitor and at least one agent selected from the group consisting of: a death receptor agonist, an autophagy inhibitor, and a NF-κB signaling pathway inhibitor, wherein the at least one mitochondrial-targeted chaperone inhibitor and the at least one agent are administered in an amount sufficient to enhance cancer or tumor cell death in a subject.

4. The method of claim 2, further comprising identifying a subject having cancer or a tumor and/or determining whether cells of said cancer or tumor have increased mitochondrial concentrations of a chaperone as compared to a control cell.

5. The method of claim 2, wherein said proliferative disorder is a cancer.

6. The method of claim 5, wherein said cancer is selected from the group consisting of small-cell lung cancer, non-small cell lung cancer, colon cancer, colorectal cancer, and pancreatic cancer.

7. The method of claim 2, wherein the mitochondrial-targeted chaperone inhibitor is a composition comprising the formula:
A-B,
wherein A is a molecular chaperone inhibitor and B is a mitochondria-penetrating moiety and A and B are linked, optionally by a linking moiety, or a pharmaceutically acceptable salt thereof.

8. The method of claim 7, wherein A is a small molecule selected from the group consisting of an Ansamycin class Hsp90 inhibitor; a geldanamycin analogue Hsp90 inhibitor; a purine-scaffold class Hsp90 inhibitor; a resorcinol Hsp90 inhibitor; and a macrolactone-Hsp90 inhibitor; or is a peptide inhibitor of Hsp90 or a Shepherdin peptide comprising SEQ ID NO:2 (His-Ser-Ser-Gly-Cys), or comprises 17-allylamino-demethoxygeldamycin (17-AAG), radicicol, a purine-scaffold class Hsp90 inhibitor, or 17-dimethylaminogeldanamycin.

9. The method of claim 8, wherein A is a peptide inhibitor of Hsp90 that comprises a sequence that is at least 95% identical to SEQ ID NO:1 and binds to and inhibits Hsp90.

10. The method of claim 7, wherein B is selected from the group consisting of: a mitochondria penetrating peptide, an RNA mitochondrial penetrating signal, guanidine-rich peptoids, guanidine-rich polycarbamates, β-oligoarginines, and proline-rich dendrimers.

11. The method of claim 10, wherein B is a mitochondria penetrating peptide selected from the group consisting of: a mitofusin peptide, a mitochondrial targeting signal peptide, TAT peptide, Antennapedia helix III homeodomain cell-penetrating peptide (ANT) peptide, VP22 peptide, and Pep-1 peptide.

12. The method of claim 7, wherein B is selected from the group consisting of: 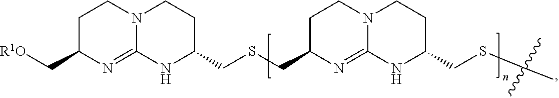
wherein:
R¹ is H, alkyl, alkenyl, alkynyl, haloalkyl, aryl, arylalkyl, or R^{a}R^{b}R^{c}Si;
R^{a}, R^{b}, and R^{c} are independently selected from alkyl or aryl; and
n is 0, 1, 2, 3, 4, 5, or 6; 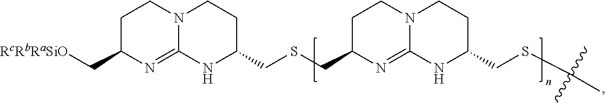
wherein:
R^{a}, R^{b}, and R^{c} are independently selected from alkyl or aryl; and
n is 1, 2, or 3;

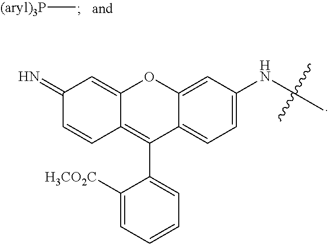

13. The method of claim 7, wherein A is 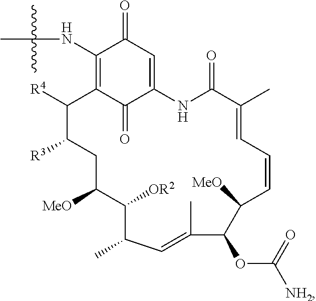
wherein:
R² is H, alkyl, aryl, or arylalkyl; R³ is H, alkyl; and R⁴ is H, alkyl, alkenyl, aryl, arylalkyl, OR^{d}, wherein R^{d} is H, alkyl, or arylalkyl.

14. The method of claim 8, wherein B comprises ANT or a mitochondrial-penetrating fragment thereof.

15. The method of claim 7, wherein the composition comprises a linking moiety between A and B.

16. The method of claim 15, wherein the linking moiety is selected from the group consisting of a peptide linker and a chemical linker.

17. The method of claim 15, wherein the linker moiety is divalent and selected from the group consisting of alkylene, alkenylene, alkynylene, cycloalkylene, arylene, heteroarylene, and peptide linker, wherein any two adjacent carbon-carbon bonds of said alkylene, alkenylene, or alkynylene, can be optionally replaced with one or more of O, NH, S, PR^{e}, C(O)NR^{f}, arylene, heterocycloalkylene, or heteroarylene; wherein Re and Rf are independently selected from alkyl or aryl.

18. The method of claim 15, wherein the linker moiety is

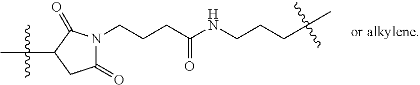

19. The method of claim 7, wherein A-B is: 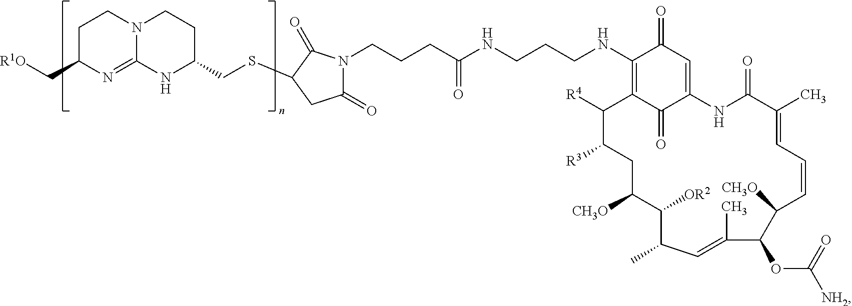
wherein, R¹ is H, alkyl, alkenyl, alkynyl, haloalkyl, aryl, arylalkyl, or R^{a}R^{b}R^{c}Si;
R² is H, alkyl, aryl, or arylalkyl; R³ is H, alkyl; R⁴ is H, alkyl, alkenyl, aryl, arylalkyl, OR^{d}, wherein R^{d} is H, alkyl, or arylalkyl;
R^{a}, R^{b}, and R^{c} are independently selected from alkyl or aryl; and
n is an integer between 1 and 10, inclusive; or a pharmaceutically acceptable salt thereof.

20. The method of claim 7, wherein A-B is selected from the group consisting of: 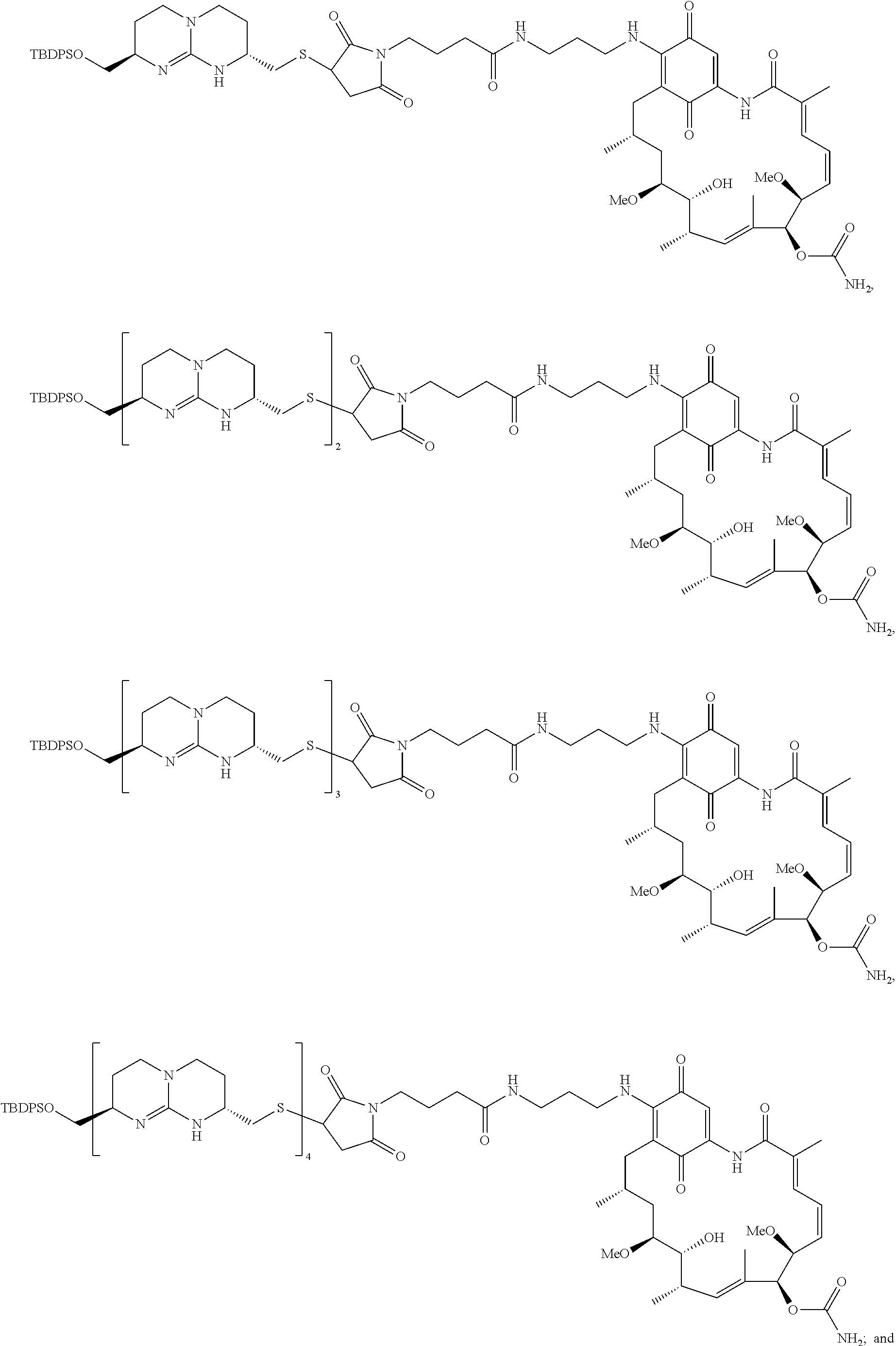 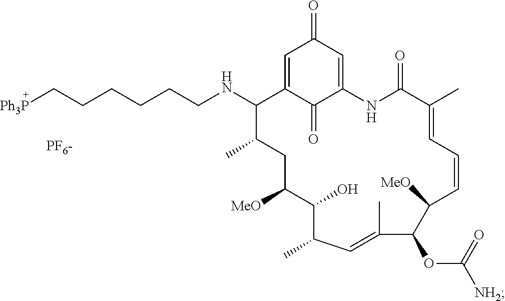
or a pharmaceutically acceptable salt thereof.

21. The method of claim 7, wherein A-B is: 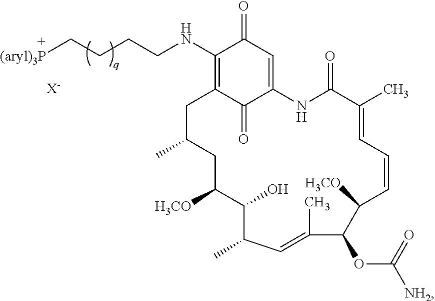
wherein, q is 1, 2, 3, 4, 5, or 6; and X is a pharmaceutically acceptable counter-ion.

22. The method of claim 2, wherein the death receptor agonist is an agonist of tumor necrosis factor (TNF)-related apoptosis-inducing ligand receptor 1 (TRAILR1), TRAILR2, CD95, tumor necrosis factor receptor 1 (TNFR1), death receptor 3 (DR3), DR6, ectodysplasin A receptor (EDAR), or nerve growth factor receptor (NGFR).

23. The method of claim 22, wherein said death receptor agonist is an agonist of TRAILR1 or TRAILR2.

24. The method of claim 23, wherein said death receptor agonist comprises an Apo2L/TRAIL polypeptide.

25. The method of claim 24, wherein said Apo2L/TRAIL is a fragment of the polypeptide of SEQ ID NO:17.

26. The method of claim 25, wherein said fragment comprises amino acids 114-281 of SEQ ID NO:17.

27. The method of claim 22, wherein the death receptor agonist comprises an antibody.

28. The method of claim 2, wherein the autophagy inhibitor is selected from the group consisting of: 3-methyladenine, bafilomycin A1, LY294002, wortmanin, hydroxychloroquine, chloroquine, 5-amino-4-imidazole carboxamide riboside, okadaic acid, a microcystin, microeystin, nodularin, analogues of cAMP, agents that elevate cAMP levels, adenosine, N6-mercaptopurine riboside, wortmannin, vinblastine, an antisense oligonucleotide, ribozyme, or siRNA that decreases the expression of MAP1LC3B, HSP90AA1, HSPA8, AMBRA1, ATG12, ATG16L1, ATG4A, ATG4B, ATG4C, ATG4D, ATG5, ATG9A, ATG9B, BECN1, GABARAP, GABARAPL1, GABARAPL2, IRGM, MAP1LC3A, RGS1, ULK1, ATG10, ATG16L1, ATG16L2, ATG3, ATG7, RAB24, DRAM, TMEM166, ATG3, AKT1, APP, ATG12, BAD, BAK1, BAX, BCL2, BCL2L1, BID, BNIP3, CASP3, CASP8, CDKN1B, CDKN2A, CLN3, CTSB, CXCR4, DAPK1, DRAM, EIF2AK3, FADD, FAS, HDAC1, HTT, IFNA2, IFNG, IGF1, INS, MAPK8, NFKB1, PIK3CG, PRKAA1, PTEN, SNCA, SQSTM1, TGFB1, TGM2, TNF, TNFSF10, CDKN1B, CDKN2A, IFNG, PTEN, RB1, TGFB1, TP53, TP73, EIF2AK3, IFNA2, IFNA4, IFNG, ARSA, CTSS, EIF4G1, ESR1, GAA, HGS, MAPK14, PIK3C3, PIK3R4, PRKAA2, RPS6KB1, TMEM74, TMEM77, ULK2, and UVRAG.

29. The method of claim 2, wherein the NF-κB signaling inhibitor reduces IκBα phosphorylation and/or degradation, NF-κB nuclear translocation, NF-κB binding to a KB promoter element, and/or transactivation of transcription of an NF-κB target gene.

30. Use of at least one mitochondrial-targeted chaperone inhibitor for the preparation of a medicament for treatment of a proliferative disorder.

31. A composition comprising at least one death receptor agonist, autophagy inhibitor, and NF-κB signaling pathway inhibitor and at least one mitochondrial-targeted chaperone inhibitor.

32. A method of treating a therapeutic-resistant cancer in a subject, said method comprising administering to a subject having cancer cells resistant to a cancer therapeutic at least one mitochondrial-targeted chaperone inhibitor and the cancer therapeutic, wherein the at least one mitochondrial-targeted chaperone inhibitor and the cancer therapeutic are administered in amount sufficient to treat the therapeutic-resistant cancer.

33. The method of claim 32, further comprising identifying a subject as having a therapeutic-resistant cancer.

34. The method of claim 33, wherein the subject is identified by detecting an increase in NF-κB signaling activity in a cancer cell in the subject.
